(19) United States
(12) Patent Application Publication
(10) Pub. No.: US 2021/0361207 A1
(43) Pub. Date: Nov. 25, 2021

(54) FLUID OPTIMIZATION AND CONTAMINANT CONTAINMENT DEVICE AND METHOD USING DISPLACEABLE PLUG

(71) Applicant: Kurin, Inc., San Diego, CA (US)

(72) Inventors: Bobby E. ROGERS, Park City, UT (US); Kevin NASON, Phoenix, AZ (US); David G. MATSUURA, Solana Beach, CA (US); Belinko K. MATSUURA, Encinitas, CA (US); Jacob A. MOEBIUS, Encinitas, CA (US); Philip J. SIMPSON, Escondido, CA (US)

(21) Appl. No.: 17/336,178

(22) Filed: Jun. 1, 2021

Related U.S. Application Data
(63) Continuation-in-part of application No. 15/855,439, filed on Dec. 27, 2017.
(60) Provisional application No. 63/033,196, filed on Jun. 1, 2020.

(51) Int. Cl. A61B 5/15

(52) U.S. Cl. CPC A61B 5/150755; A61B 5/15003; A61B 5/1405; A61B 2560/04; A61B 5/150351; A61B 5/150572; A61B 5/150343

(57) ABSTRACT

A fluid sample optimization device for optimizing a fluid sample collected by a fluid collection device from a fluid source, where a first portion of the fluid sample potentially has contaminants. The device includes an inlet configured to connect with the fluid source, an outlet configured to connect with the fluid collection device, a sample path connected between the inlet and the outlet, and a contaminant containment reservoir connected between the inlet and the outlet. The contaminant containment reservoir has an air permeable fluid resistor proximate the outlet, and is arranged to receive the first portion of the fluid sample from the fluid source to displace air therein, such that upon receipt of the first portion of the fluid sample and containment of the contaminants in the contaminant containment reservoir, subsequent portions of the fluid sample are conveyed by the sample path from the inlet to the outlet when subsequent pressure differentials are applied between the inlet and the outlet. The fluid sample optimization device can further include a displaceable plug between the inlet and the sample path, that can be displaced by the subsequent pressure differentials to allow the subsequent portions of the fluid to be conveyed through the sample path.

### CROSS-REFERENCE TO RELATED APPLICATIONS

This application claims the benefit of U.S. Provisional Application No. 63/033,196, filed Jun. 1, 2020 and is a continuation-in-part of U.S. application Ser. No. 15/855,439, filed Dec. 27, 2017, both of which are application is incorporated herein by reference in its entirety.

### BACKGROUND

Bacteraemia is the presence of microorganisms in the blood. Sepsis, on the other hand, is bacteraemia in the presence of clinical symptoms and signs such as fever, tachycardia, tachypnea and hypotension. Bacteraemia and sepsis are associated with a high mortality and an increased incidence and duration of hospital stay and associated costs. Many bacteraemias, sepsis, fungaemias and other pathogens actually occur within a hospital or other healthcare settings with catheters and venipunctures being a source of contamination as potential carriers of these pathogens.

Blood cultures are the standard test used to detect microbial pathogens related to bacteraemia and sepsis in a patient's blood. The term blood culture refers to a single venipuncture, either from a peripheral site or central or arterial line, with the blood inoculated into one or more blood culture bottles or containers. One bottle is considered a blood culture where two or more are considered a set. Multiple sets may be obtained from multiple venipunctures and are associated with different sites on the patient.

These methods allow for microbial identification and susceptibility testing to be performed, which is a critical component to managing sepsis, however the lack of rapid results and decreased sensitivity for fastidious pathogens has led to the development of improved systems and adjunctive molecular or proteomic testing.

Collection of blood samples for conducting blood cultures is a critical component of modem patient care and can either positively affect the patient outcome by providing an accurate diagnosis, or can adversely affect the outcome by prolonging unnecessary antimicrobial therapy, the length of hospital stays, and increasing costs.

One outcome of collection of blood cultures is contamination. Blood culture contamination can lead to a false positive culture result and/or significant increase in healthcare related costs. Sources of blood culture contamination include improper skin antisepsis, improper collection tube disinfection, and contamination of the initial blood draw which may then skew results.

Blood culture collection kits generally consist of a “butterfly” set, infusion set, or other type of venipuncture device as offered by companies like BD, Smiths, B. Braun and others, and aerobic and anaerobic blood culture bottles. Various different bottles are also available depending on the test requirements. These bottles are specifically designed to optimize recovery of both aerobic and anaerobic organisms. In conventional kits, a bottle used is known generally as a “Vacutainer,” which is a blood collection tube formed of a sterile glass or plastic tube with a closure that is evacuated to create a vacuum inside the tube to facilitate the draw of a predetermined volume of liquid such as blood.

False positive blood cultures are typically a result of poor sampling techniques. They cause the use of antibiotics when not needed, increasing hospital costs and patient anxiety. Blood cultures are drawn from a needlestick into the skin, and then a Vacutainer is attached to capture a sample of blood. Contamination may occur from improper or incomplete disinfection of the skin area in and around the puncture site. It may also occur from the coring of the skin by the needle during insertion, with the cored skin cells and any associated contamination being pulled into the sample.

Blood flow through a hypodermic needle is laminar, and as such, a velocity gradient can be developed over the flow tube as a pressure drop is applied to the hypodermic needle. Either forceful aspiration of blood, or using a very small hypodermic needle, can cause lysis and a release of potassium from the red blood cells, thereby rendering the blood samples abnormal.

In other instances, some patients have delicate veins that can collapse under a pressure drop or vacuum, particularly as applied by a syringe's plunger that is drawn too quickly for the patient's condition. Since such condition is impossible to know beforehand, such vein collapses are a risk and very difficult to control.

Various strategies have been implemented to decrease blood culture contamination rates, e.g. training staff with regard to aseptic collection technique, feedback with regard to contamination rates and implementation of blood culture collection kits. Although skin antisepsis can reduce the burden of contamination, 20% or more of skin organisms are located deep within the dermis and are unaffected by antisepsis. Changing needles before bottle inoculation is not advisable as it increases the risk to acquire needle stick injuries without decreasing contamination rates.

Some conventional systems and techniques for reducing blood culture contamination include discarding the initial aliquot of blood taken from central venous catheters, venipunctures, and other vascular access systems. However, these systems require the user to mechanically manipulate an intravascular device, or require a complex series of steps that are difficult to ensure being followed.

Recent innovations have proposed novel approaches to reduce blood contaminants by utilizing methods based on U.S. Pat. No. 9,820,682. The '682 patent utilized the patient's own blood pressure to manage blood contamination by allowing the initial aliquot of blood to flow into a channel that vents to atmosphere. While this approach works well, if a patient's blood pressure is too low it can lead to long fill times of the contaminant containment reservoir.

Another approach disclosed in US Patent Publication No. 2019/0365303, which appears inspired by the concepts of the '682 patent, makes use of vacuum pressure from a syringe or vacuum bottle, and dissolving membranes, flow controllers or flow restrictors, and other mechanical moving parts to reduce blood sample contamination. This approach, while possibly eliminating extended fill times of the contaminant containment reservoir that may occur with reliance on patient blood pressure as the driving mechanism, presents other problems in the second channel, the sampling channel. First, dissolving materials may impact sample test results and understanding all the potential testing variations that may occur is difficult to assess. Second, flow controllers or flow restrictors as described in the '303 publication impede flow, and such restrictions may create hemolysis which can negatively impact test results. Further, flow restrictions come with a potential addition of wait time to fill a fluid collection device, which is also undesirable.

### SUMMARY

This document describes a non-venting bodily fluid sample optimization device and system, for use in a blood sampling or blood culture collection system. In accordance with implementations described herein, a device has no permanently-attached, statically positioned moving parts, such as valves, state-transitioning switches or diverters, or other mechanisms that move, shift or transition from one operating mode to another operating mode, or from one state to another state.

In one aspect, a fluid sample optimization device is described for optimizing a fluid sample collected by a fluid collection device from a fluid source, where a first portion of the fluid sample potentially has contaminants. The fluid sample optimization device includes an inlet configured to connect with the fluid source, an outlet configured to connect with the fluid collection device, and a sample path connected between the inlet and the outlet. The fluid sample optimization device further includes a contaminant containment reservoir connected between the inlet and the outlet. The contaminant containment reservoir has an air permeable fluid resistor proximate the outlet, and is arranged to receive, when a pressure differential is applied between the inlet and the outlet, a first portion of the fluid sample from the fluid source to displace air therein through the air permeable fluid resistor and the outlet, such that upon receipt of the first portion of the fluid sample and containment of the contaminants in the contaminant containment reservoir, subsequent portions of the fluid sample can be conveyed by the sample path from the inlet to the outlet when subsequent pressure differentials are applied between the inlet and the outlet. The fluid sample optimization device can further include a displaceable plug between the inlet and the sample path, or in the sample path, that can be displaced by the subsequent pressure differentials to allow the subsequent portions of the fluid to be conveyed through the sample path.

In another aspect, a fluid sample optimization device includes an inlet configured to connect with the fluid source, and an outlet configured to connect with the fluid collection device that provides a negative pressure differential between the inlet and the outlet. The fluid sample optimization device further includes a sample path connected between the inlet and the outlet, a junction between the inlet and the sample path having a displaceable plug that is configured to inhibit at least a part of the first portion of the fluid sample and the contaminants from entering the sample path. The fluid sample optimization device further includes a contaminant containment reservoir connected between the inlet and the outlet, and that includes an air permeable fluid resistor proximate the outlet. The contaminant containment reservoir is arranged to receive, when a pressure differential is applied between the inlet and the outlet, the first portion of the fluid sample from the fluid source to displace air therein through the air permeable fluid resistor and the outlet, such that upon receipt of the first portion of the fluid sample and containment of the contaminants in the contaminant containment reservoir, subsequent portions of the fluid sample can move the displaceable plug and be conveyed by the sample path from the inlet to the outlet when subsequent pressure differentials are applied between the inlet and the outlet.

The details of one or more embodiments are set forth in the accompanying drawings and the description below. Other features and advantages will be apparent from the description and drawings, and from the claims.

### BRIEF DESCRIPTION OF THE DRAWINGS

These and other aspects will now be described in detail with reference to the following drawings.

FIG. 1 illustrates a blood sample optimization system.

FIG. 2 illustrates a blood sample optimization system in accordance with an alternative implementation.

FIG. 3 illustrates a blood sample optimization system in accordance with another alternative implementation.

FIG. 4 illustrates a blood sample optimization system in accordance with another alternative implementation.

FIG. 5 illustrates a blood sample optimization system in accordance with another alternative implementation.

FIG. 6 illustrates a blood sample optimization system in accordance with an alternative implementation.

FIG. 7 is a flowchart of a method for optimizing a quality of a blood culture.

FIGS. 8A-8E illustrate a blood sequestration system for non-contaminated blood sampling, in accordance with some implementations.

FIG. 9 illustrates a pathway splitter for use in a blood sequestrations system.

FIGS. 10A-10D illustrate a blood sequestration system for non-contaminated blood sampling, in accordance with alternative implementations.

FIGS. 11A-11E illustrate a blood sequestration system for non-contaminated blood sampling, in accordance with other alternative implementations.

FIGS. 12A-12D illustrate a blood sample optimization system including a blood sequestration device in accordance with yet other alternative implementations.

FIGS. 13A-13D illustrate a blood sample optimization system 1300 in accordance with yet another alternative implementations.

FIGS. 14A-14E illustrate yet another implementation of a blood sampling system to sequester contaminates of an initial aliquot or sample to reduce false positives in blood cultures or tests performed on a patient's blood sample.

FIGS. 15A-15G illustrate a blood sequestration device and method of using the same, in accordance with yet another implementation.

FIGS. 16A-16D illustrate a blood sequestration device in accordance with yet another implementation.

FIGS. 17A-17E illustrate a bottom member of a housing for a blood sequestration device.

FIGS. 18A-18F illustrate a top member of a housing for a blood sequestration device.

FIGS. 19A and 19B illustrate a blood sequestration device having a top member mated with a bottom member.

FIG. 20 shows a blood sample optimization system including a blood sequestration device.

FIG. 21 illustrates a non-vented blood sequestration device using a wicking material chamber.

FIGS. 22A and 22B illustrate a material makeup of a filter for sequestering blood in a sequestration chamber of a blood sequestration device.

FIGS. 23A-23E illustrate another implementation of a blood sequestration device that uses a vacuum force from a blood collection device.

FIGS. 24A-24D illustrate another implementation of a blood optimization system and blood sequestration device.

FIGS. 25A-25D illustrate another implementation of a blood optimization system and blood sequestration device.

FIGS. 26A-26E illustrate another implementation of a blood optimization system and blood sequestration device.

FIGS. 27A-27D illustrate another implementation of a blood optimization system and blood sequestration device.

FIGS. 28A-28F illustrate another implementation of a blood optimization system and blood sequestration device.

FIGS. 29A-29C illustrate another implementation of a blood optimization system and blood sequestration device.

FIGS. 30A-30G illustrate another implementation of a blood optimization system and blood sequestration device.

FIG. 31 illustrates a non-venting fluid contaminant sample optimization devices, in accordance with implementations described herein;

FIGS. 32A-32C illustrate a fluid sample optimization device having a housing, an air-permeable fluid barrier, and a displaceable plug, consistent with implementations described herein.

FIGS. 33A-33D illustrate a fluid sample optimization device consistent with implementations described herein.

FIGS. 34A-34C illustrate various alternative implementations of a displaceable plug or stopper, shown in the form of a ball or rounded object.

FIGS. 35A and 35B illustrate various alternative implementations of a displaceable plug or stopper, shown in the form of a disk.

FIGS. 36A-36C illustrate further various alternative implementations of a displaceable plug or stopper, consistent with the devices described herein.

FIGS. 37A and 37B show a variation of a fluid path for fluid flow after displacement of a plug; and

FIGS. 38A-38C illustrate another fluid sample optimization device consistent with implementations described herein.

Like reference symbols in the various drawings indicate like elements.

### DETAILED DESCRIPTION

This document describes fluid sample optimization systems and methods for reducing or eliminating contaminates in collected blood samples, which in turn reduces or eliminates false positive readings in blood cultures or other testing of collected blood samples. In some implementations, a blood sample optimization system includes a patient needle for vascular access to a patient's bloodstream, a sample needle for providing a blood sample to a blood collection container, such as an evacuated blood collection container or tube like a Vacutainer™ or the like, or other sampling device, and a fluid sample optimization device, for containing possible contaminants in a first amount of a fluid sample, such as a blood sample. Subsequent amounts of the fluid sample are allowed to bypass the first amount, thereby containing any contaminants in the first amount while providing less to zero contaminates in fluid samples in the subsequent amounts of the fluid.

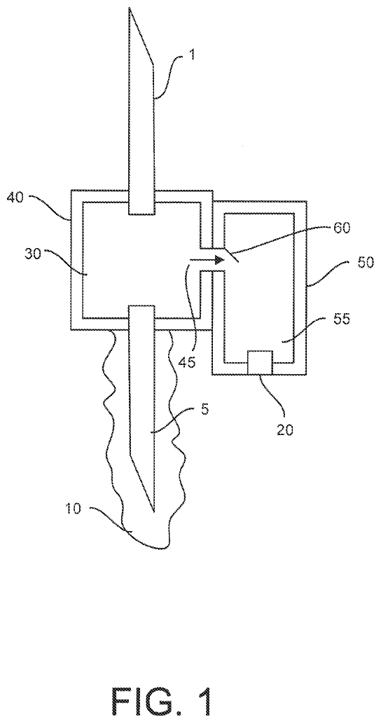

FIG. 1 illustrates a blood sample optimization system in accordance with some implementations. The system includes a patient needle 1 to puncture the skin of a patient to access the patient's vein and blood therein. The system further includes a sample needle (i.e., a resealably closed needle for use with Vacutainers™ or the like) 5, which may be contained within and initially sealed by a resealable boot 10, a Luer activated valve, or another collection interface or device. The resealable boot 10 can be pushed aside or around the sample needle 5 by application of a Vacutainer™ bottle (not shown) for drawing the patient's blood. The system can further include a low volume chamber 30 that leads to the sample needle 5, but also includes an orifice or one or more channels 45 that lead to a sequestration chamber 55 formed by a housing 50.

The sequestration chamber 55 is a chamber, channel, pathway, lock, or other structure for receiving and holding a first aliquot of the patient's blood, which may be in a predetermined or measured amount, depending on a volume of the sequestration chamber 55. The first draw of blood typically contains or is more susceptible to containing organisms that cause bacteraemia and sepsis or other pathogens than subsequent blood draws. The sequestration chamber 55 can be a vessel encased in a solid housing, formed in or defined by the housing itself, or can be implemented as tubing or a lumen. The sequestration chamber 55, regardless how formed and implemented, may have a predetermined volume. In some implementations, the predetermined volume may be based on a volume of the patient needle, i.e. ranging from less than the volume of the patient needle to any volume up to or greater than 20 times or more of the volume of the patient needle. The predetermined volume of the sequestration chamber 55 may also be established to economize or minimize an amount of blood to be sequestered and disposed of.

The sequestration chamber 55 can be formed, contained or housed in a chamber housing 50, and can be made of plastic, rubber, steel, aluminum or other suitable material. For example, the sequestration chamber 55 could be formed of flexible tubing or other elastomeric materials. The sequestration chamber 55 further includes an air permeable blood barrier 20 that allows air to exit the sequestration chamber 55. As used herein the term “air permeable blood barrier” means an air permeable but substantially blood impermeable substance, material, or structure. Examples may include hydrophobic membranes and coatings, a hydrophilic membrane or coating combined with a hydrophobic membrane or coating, mesh, a filter, a mechanical valve, antimicrobial material, or any other means of allowing air to be displaced from the sequestration chamber 55 as it is filled with blood. In various exemplary embodiments, an air permeable blood barrier may be formed by one or more materials that allow air to pass through until contacted by a liquid, such material then becomes completely or partially sealed to prevent or inhibit the passage of air and/or liquid. In other words, prior to contact with liquid, the material forms a barrier that is air permeable. After contact with a liquid, the material substantially or completely prevents the further passage of air and/or liquid.

The orifice or channel 45 can be any desired length, cross-sectional shape or size, and/or can be formed to depart from the low volume chamber 30 at any desired angle or orientation. The orifice or channel 45 may also include a one-way flap or valve 60 that maintains an initial aliquot of blood sample within the sequestration chamber 55. In some specific implementations, the orifice or channel 45 can include a “duck bill” or flapper valve 60, or the like, for one-way flow of blood from low volume chamber 30 to the sequestration chamber 55. The air permeable blood barrier 20 can also be constructed of a material that allows air to exit but then seals upon contact with blood, thereby not allowing external air to enter sequestration chamber 55. This sealing would eliminate the need for a valve.

Valve 60 can be any type of valve or closing mechanism. Chamber 30 is designed to hold virtually no residual blood, and can be designed to be adapted to hold or allow pass-through of a particular volume or rate of blood into sequestration chamber 55. Likewise, sequestration chamber 55 may also include any type of coating, such as an antimicrobial coating, or a coating that aids identification and/or diagnosis of components of the first, sequestered blood draw.

Housing 50 and 40 can be formed of any suitable material, including plastic, such as acrylonitrile butadiene styrene (ABS) or other thermoplastic or polymeric material, rubber, steel, or aluminum. The air permeable blood barrier 20 can include a color-providing substance, or other signaling mechanism, that is activated upon contact with blood from the initial blood draw, or when air displacement is stopped, or any combination of events with blood in the sequestration chamber 55. The air permeable barrier may also include an outer layer such as a hydrophobic membrane or cover that inhibits or prevents the inadvertent or premature sealing of the filter by an external fluid source, splash etc. Sequestration chamber 55 can also be translucent or clear to enable a user to visually confirm the chamber is filled.

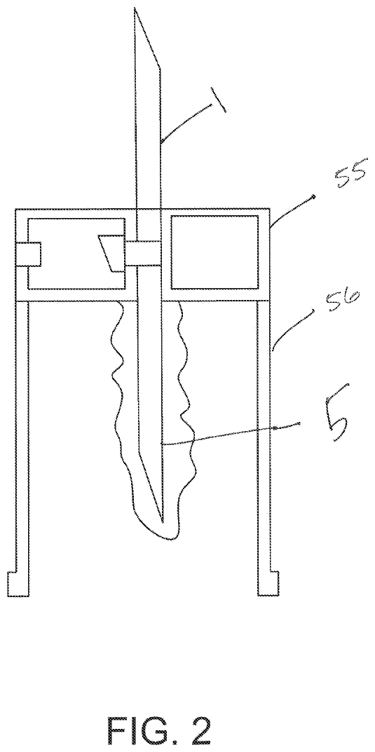

FIG. 2 illustrates a blood sample optimization system in accordance with some alternative implementations. In the implementation shown in FIG. 2, a sequestration chamber 55, or waste chamber, surrounds the patient needle 1, with an open-ended cuff or housing connected with the waste chamber and encircling the sample needle housing base and housing. The patient needle 1 and sample needle 5 are connected together by a boot 56, which forms a continuous blood draw channel therethrough. The boot 56 includes a single orifice or channel leading from the blood draw channel into sequestration chamber 55. The device can include more than one single orifice or channel, in other implementations. Each orifice or channel can include a one-way valve, and can be sized and adapted for predetermined amount of blood flow.

The sequestration chamber 55 includes an air permeable blood barrier. The filter can further include a sensor or indicator to sense and/or indicate, respectively, when a predetermined volume of blood has been collected in the sequestration chamber 55. That indication will alert a user to attach an evacuated blood collection tube or bottle, such as a Vacutainer™ to the sample needle 5. The housing for the sequestration chamber 55 can be any size or shape, and can include any type of material to define an interior space or volume therein. The interior space is initially filled only with air, but can also be coated with an agent or substance, such as a decontaminate, solidifying agent, or the like. Once evacuated blood collection tube is attached to the sample needle 5, blood will flow automatically into the patient needle 1, through the blood draw channel and sample needle 5, and into the bottle. The sample needle 5 is covered by a resealable boot, coating or membrane that seals the sample needle when a blood collection bottle is not attached thereon or thereto.

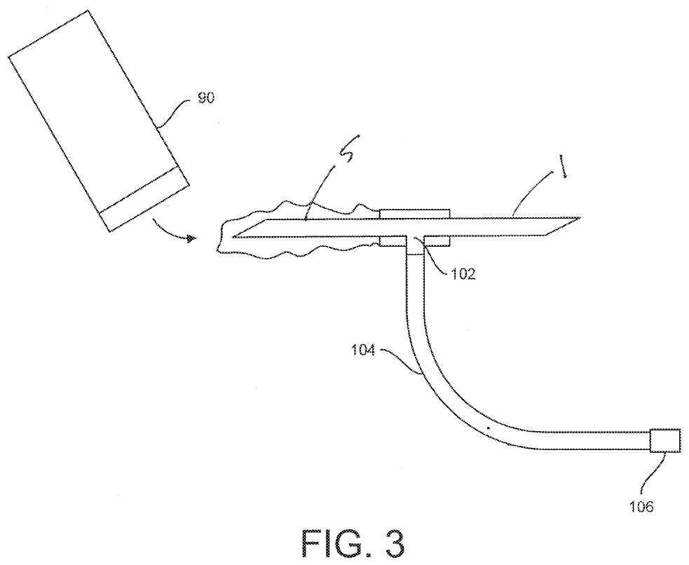

FIG. 3 illustrates a blood sample optimization system in accordance with some alternative implementations. In the implementation shown, a sample needle 5 is surrounded by a resealable boot or membrane, and is further connected with a patient needle 1. A blood flow channel is formed through the sample needle and the patient needle. The connection between the sample needle and patient needle includes a “T” or “Y” connector 102, which includes a channel, port or aperture leading out from the main blood flow channel to a sequestration chamber 104.

The T or Y connector 102 may include a flap or one-way valve, and have an opening that is sized and adapted for a predetermined rate of flow of blood. The sequestration chamber 104 can be formed from tubing, or be formed by a solid housing, and is initially filled with air. The sequestration chamber 104 will receive blood that flows out of a patient automatically, i.e. under pressure from the patient's own blood pressure. The sequestration chamber 104 includes an air permeable blood barrier 106, preferably at the distal end of tubing that forms the sequestration chamber 104, and which is connected at the proximal end to the T or Y connector 102. The T or Y connector 102 can branch off at any desired angle for most efficient blood flow, and can be formed so as to minimize an interface between the aperture and channel and the main blood flow channel, so as to minimize or eliminate mixing of the initial aliquot of blood with main blood draw samples.

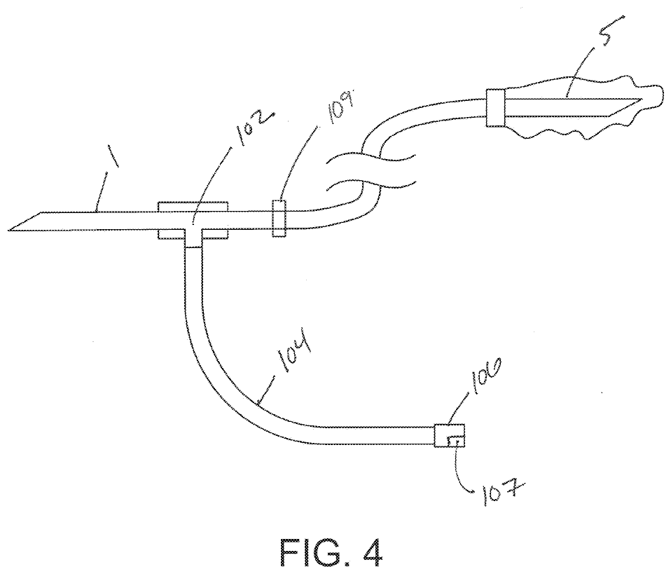

In some alternative implementations, the sample needle may be affixed to a tubing of any length, as shown in FIG. 4, connecting at its opposite end to the T or Y connector 102. The sequestration chamber 104 can be any shape or volume so long as it will contain a predetermined amount of blood sample in the initial aliquot. The T or Y connector 102 may also include an opening or channel that is parallel to the main blood flow channel. The air permeable blood barrier may further include an indicator 107 or other mechanism to indicate when a predetermined amount of blood has been collected in the sequestration chamber, or when air being expelled reaches a certain threshold, i.e. to zero. The tubing can also include a clip 109 that can be used to pinch off and prevent fluid flow therethrough.

Once the air permeable blood barrier and primary chamber are sealed the initial aliquot of blood is trapped in the sequestration chamber 104, an evacuated blood collection tube, such as a Vacutainer™ bottle may be attached to the sample needle 5 to obtain the sample. The blood collection tube can be removed, and the sample needle 5 will be resealed. Any number of follow-on blood collection tubes can then be attached for further blood draws or samples. Upon completion of all blood draws, the system can be discarded, with the initial aliquot of blood remaining trapped in the sequestration chamber 104.

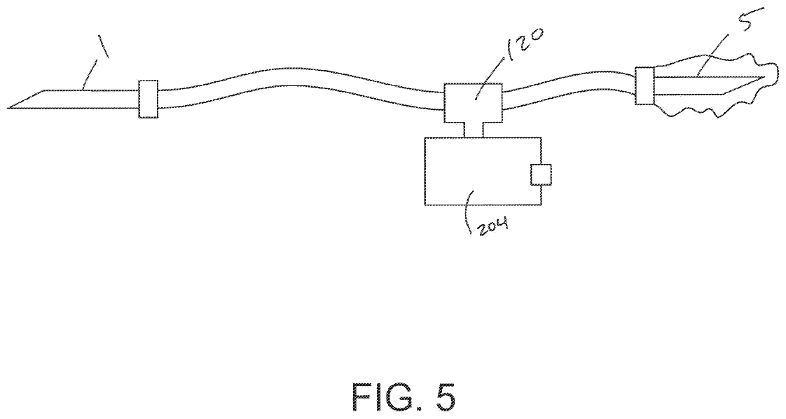

FIG. 5 illustrates a blood sample optimization system in accordance with some alternative implementations. In the implementation shown, a sample needle 5 is connected with a patient needle by tubing. A “T” or “Y” connector 120 is added along the tubing at any desired location, and includes an aperture, port or channel leading to a sequestration chamber 204, substantially as described above.

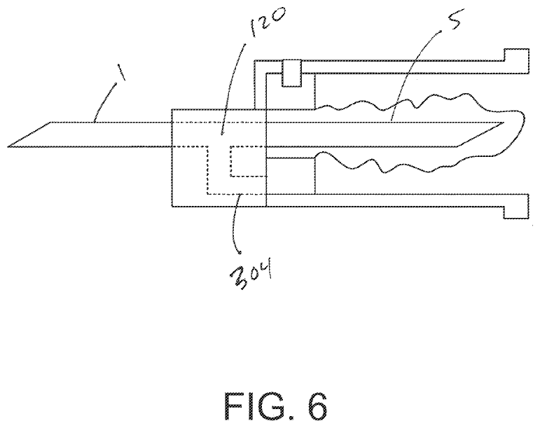

FIG. 6 illustrates a blood sample optimization system in accordance with some alternative implementations, in which a sequestration chamber 304, formed as a primary collection channel, receives an initial aliquot of blood, and is provided adjacent to the blood sampling channel. The sequestration chamber 304 can encircle the blood sampling channel, the patient needle 1, and/or the sample needle 5. The primary collection channel can include a T or Y connector 120, or other type of aperture or channel. The sequestration chamber 304 includes an air permeable blood barrier, which can also include an indicator of being contacted by a fluid such as blood, as described above.

In some implementations, either the patient needle 1 or the sample needle 5, or both, can be replaced by a Luer lock male or female connector. However, in various implementations, the connector at a sample needle end of the blood sample optimization system is initially sealed to permit the diversion of the initial aliquot of blood to the sequestration chamber, which is pressured at ambient air pressure and includes the air outlet of the air permeable blood barrier. In this way, the system passively and automatically uses a patient's own blood pressure to overcome the ambient air pressure of the sequestration chamber to push out air through the air permeable blood barrier and displace air in the sequestration chamber with blood.

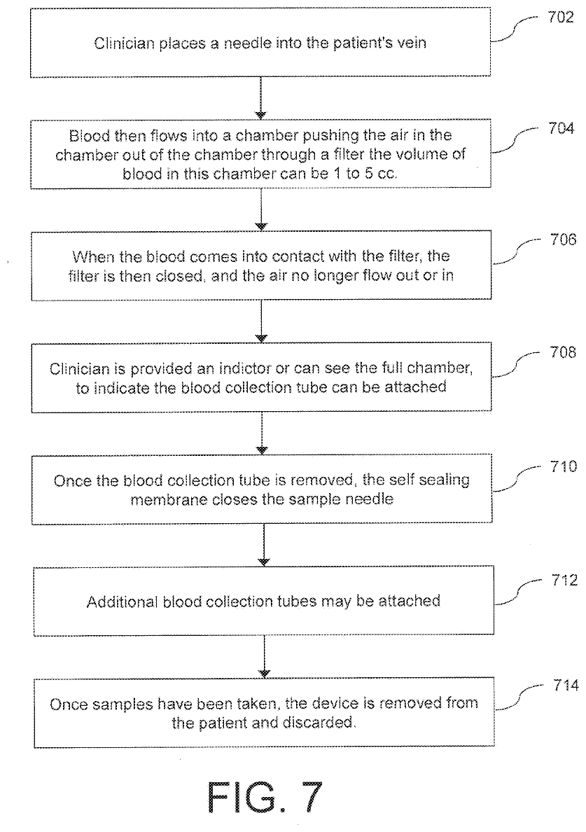

FIG. 7 is a flowchart of an exemplary method for optimizing the quality of a blood culture. At 702, a clinician places a needle into a patient's vein. At 704, blood then flows into a sequestration chamber, pushing the air in the sequestration chamber out of the sequestration chamber through an air permeable blood barrier. In some implementations, the volume of the sequestration chamber is less than 0.1 to more than 5 cubic centimeters (cc's), or more. The sequestration chamber is sized and adapted to collect a first portion of a blood sample, which is more prone to contamination than secondary and other subsequent portion of the blood sample or subsequent draws. Since the sequestration chamber has an air-permeable blood barrier through which air can be displaced by blood pushed from the patient's vein, such blood will naturally and automatically flow into the sequestration chamber before it is drawn into or otherwise enters into a Vacutainer or other bottle for receiving and storing a blood sample.

When the sequestration chamber fills, the blood will gather at or otherwise make contact with the air permeable blood barrier, which will inhibit or prevent blood from passing therethrough. At 706, when the blood comes into contact with the entire internal surface area of the air permeable blood barrier, the air permeable blood barrier is then closed and air no longer flows out or in. At 708, the clinician may be provided an indictor or can see the full chamber, to indicate the evacuated blood collection tube, such as a Vacutainer™ can be attached. The indicator can include visibility into the primary chamber to see whether it is full, the blood barrier changing color, for example, or other indicator. The fill time of the sequestration chamber may be substantially instantaneous, so such indicator, if present, may be only that the sequestration chamber is filled.

Prior to an evacuated blood collection tube being attached, communication between the needle, sampling channel, and the sequestration chamber is restricted by the sealing of the sequestration chamber blood barrier thereby not permitting air to reenter the system through the sequestration. Sealing the communication path could also be accomplished with a mechanical twist or other movement, a small orifice or tortuous pathway, eliminating the need for a separate valve or mechanical movement or operation by the clinician. At 710, once the evacuated blood collection tube is removed, the self-sealing membrane closes the sample needle, and at 712, additional subsequent evacuated blood collection tubes may be attached. Once samples have been taken, at 714 the device is removed from the patient and discarded.

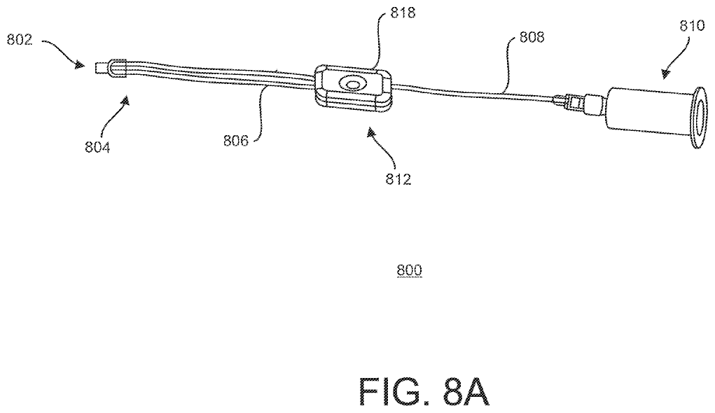

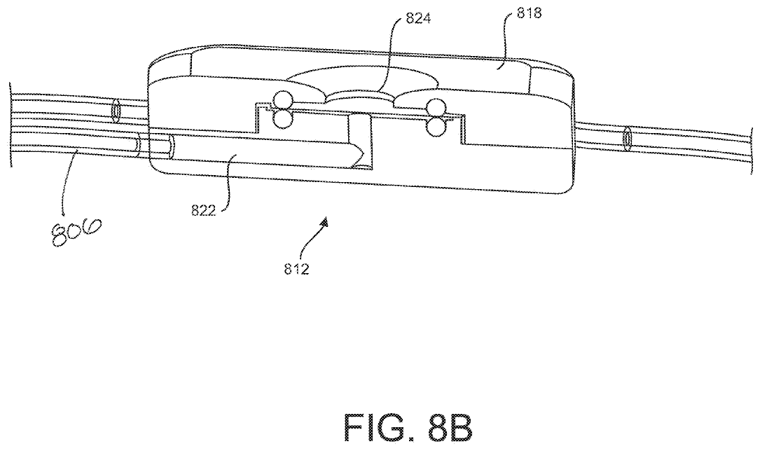

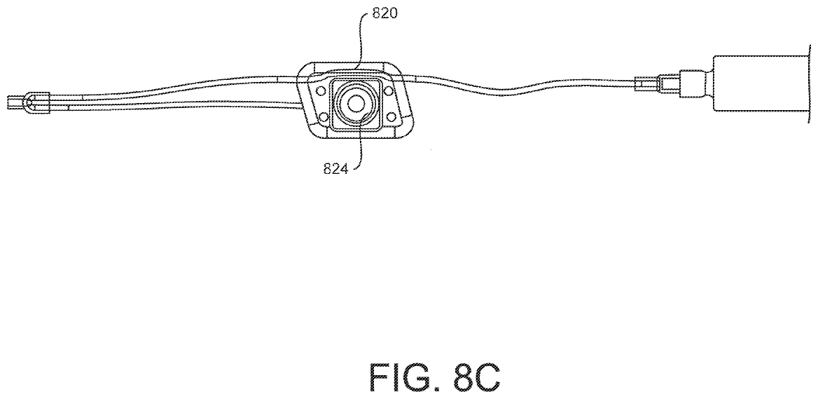

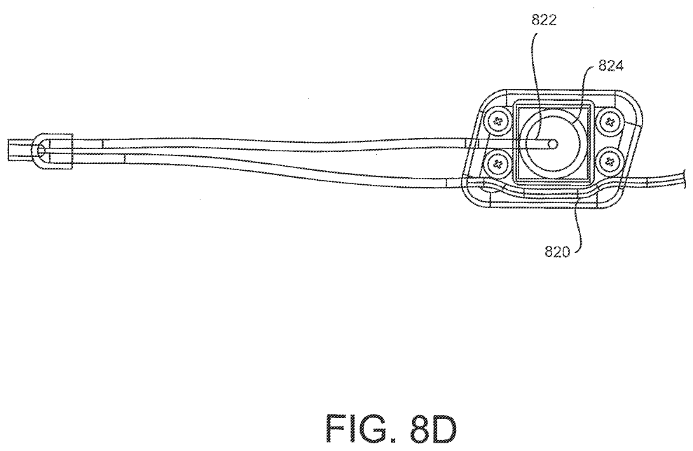

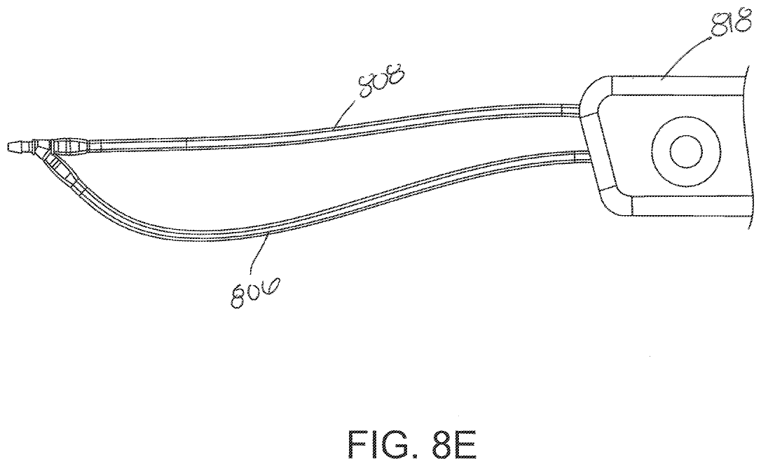

FIGS. 8A-8E illustrate an exemplary blood sample optimization system 800 for non-contaminated blood sampling, in accordance with some implementations. The blood sample optimization system 800 includes an inlet port 802 that can be connected to tubing, a patient needle (or both), or other vascular or venous access device, and a pathway splitter 804 having a first outlet to a sequestration chamber tubing 806 and a second outlet to sample collection tubing 808. One or both of the sequestration chamber tubing 806 and the sample collection tubing 808 can be formed of tubing. In some implementations, the sequestration chamber tubing 806 is sized so as to contain a particular volume of initial blood sample. The sample collection tubing 808 will receive a blood sample once the sequestration chamber tubing 806 is filled. The sample collection tubing 808 can be connected to a Vacutainer™ base or housing 810, or other blood sample collection device.

The blood sequestration system 800 further includes a blood sequestration device 812 which, as shown in more detail in FIGS. 8B-8D, includes a housing 818 that includes a sampling channel 820 defining a pathway for the non-contaminated sample collection tubing 808 or connected at either end to the non-contaminated sample collection tubing 808. The sampling channel 820 can be curved through the housing 818 so as to better affix and stabilize the housing 818 at a location along the non-contaminated sample collection tubing 808.

The blood sequestration device 812 further includes a sequestration chamber 822 connected with the sequestration chamber tubing 806 or other chamber. The sequestration chamber 822 terminates at an air permeable blood barrier 824. The air permeable blood barrier 824 can also include a coloring agent that turns a different color upon full contact with blood, as an indicator that the regular collection of blood samples (i.e. the non-contaminated blood samples) can be initiated. Other indicators may be used, such as a small light, a sound generation mechanism, or the like. In some implementations, the air permeable blood barrier is positioned at a right angle from the direction of sequestration chamber 822, but can be positioned at any distance or orientation in order to conserve space and materials used for the housing 818. The housing 818 and its contents can be formed of any rigid or semi-rigid material or set of materials.

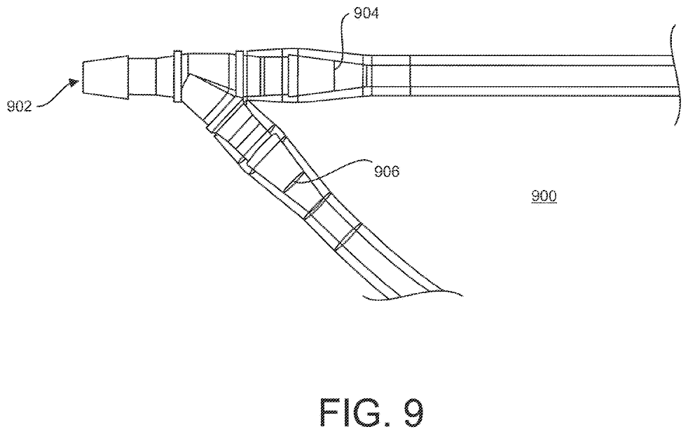

FIG. 9 illustrates a pathway splitter 900 for use in a blood sequestrations system, such as those shown in FIGS. 8A-8E, for example. The pathway splitter 900 includes an inlet port 902, a main line outlet port 904, and a sequestration channel outlet port 906. The inlet port 902 can be connected to main tubing that is in turn connected to a patient needle system, or directly to a patient needle. The main line outlet port 904 can be connected to main line tubing to a blood sampling system, such as a vacutainer base or housing, or directly to such blood sampling system. The sequestration channel outlet port 906 can be connected to sequestration tubing for receiving and sequestering a first sample of blood, up to a measured amount or predetermined threshold. Alternatively, the sequestration channel outlet port 906 can be connected to a sequestration chamber. The sequestration channel outlet port 906 is preferably 20-70 degrees angled from the main line outlet port 904, which in turn is preferably in-line with the inlet port 902. Once the predetermined amount of initial blood sample is sequestered in the sequestration tubing or chamber, in accordance with mechanisms and techniques described herein, follow-on blood samples will flow into the inlet port 902 and directly out the main line outlet port 904, without impedance.

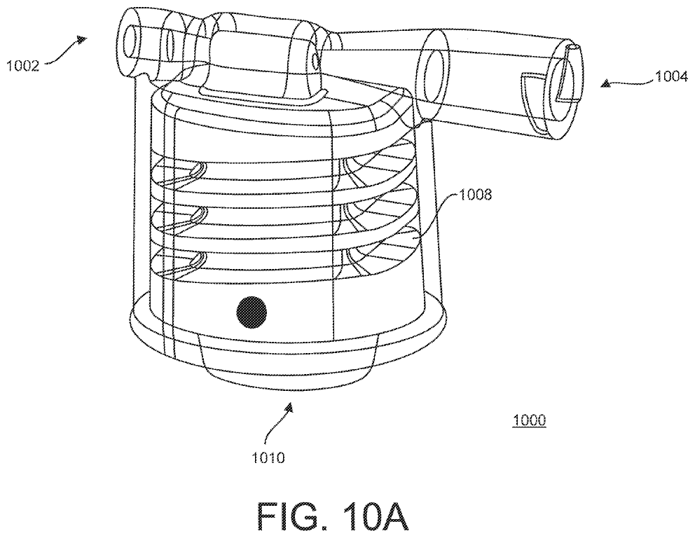

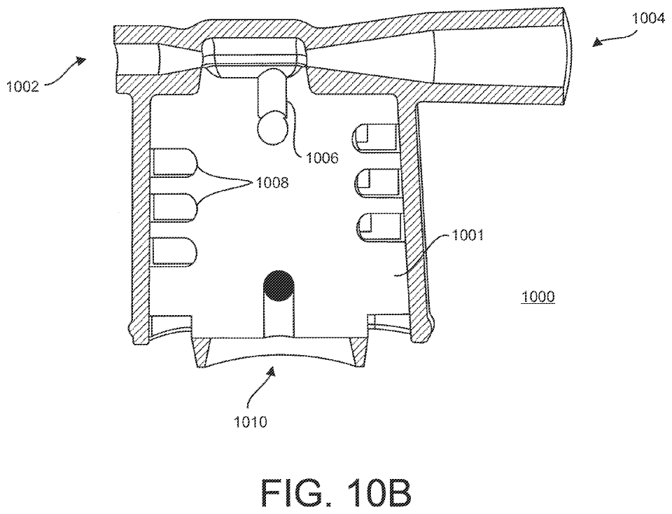

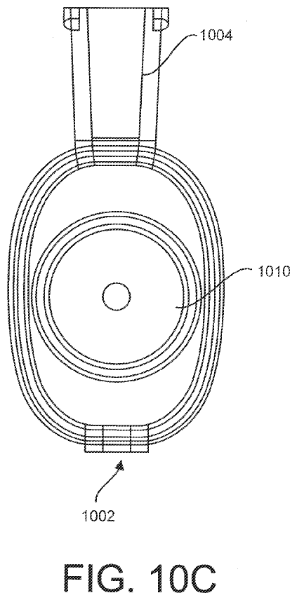

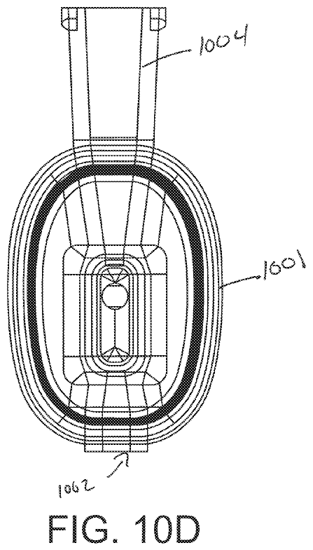

FIGS. 10A-10D illustrate a blood sequestration device 1000 in accordance with alternative implementations. The blood sequestration device 1000 includes an inlet port 1002, a main outlet port 1004, and a sequestration channel port 1006. The inlet port 1002 can be connected to a patient needle or related tubing. The main outlet port 1004 can be connected to a blood sample collection device such as a Vacutainer, associated tubing, or a Luer activated valve, or the like. The sequestration channel port 1006 splits off from the main outlet port 1004 to a sequestration chamber 1008. In some implementations, the sequestration chamber 1008 is formed as a helical channel within a housing or other container 1001.

The sequestration chamber 1008 is connected at the distal end to an air permeable blood barrier 1010, substantially as described above. Air in the sequestration chamber 1008 is displaced through the air permeable blood barrier 1010 by an initial aliquot of blood that is guided into the sequestration channel port 1006. Once the sequestration chamber 1008 is filled, further blood draws through the main outlet port 1004 can be accomplished, where these samples will be non-contaminated.

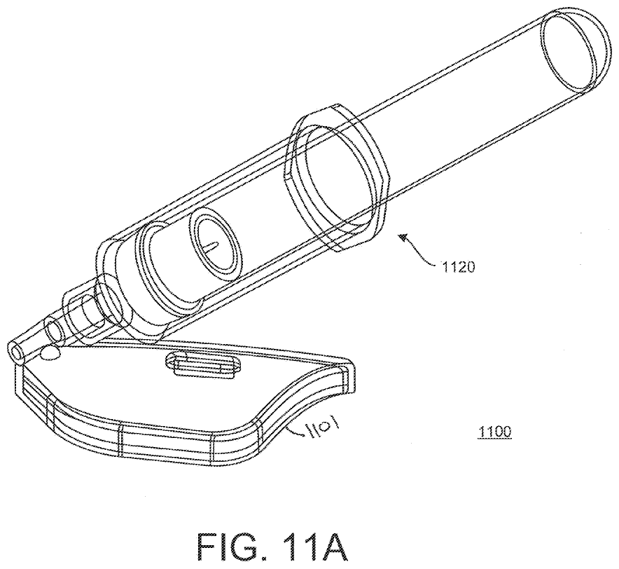

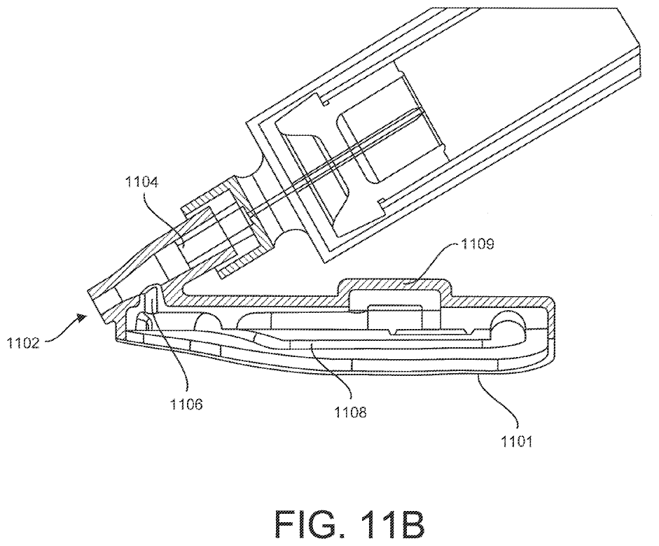

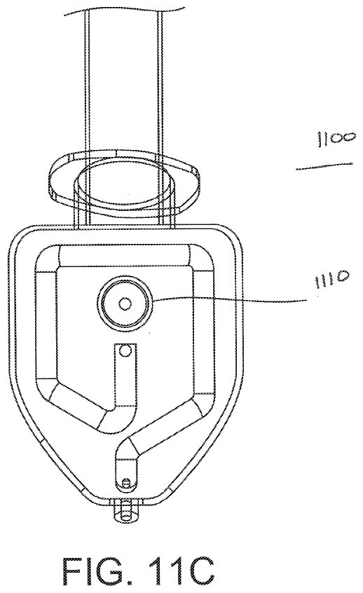

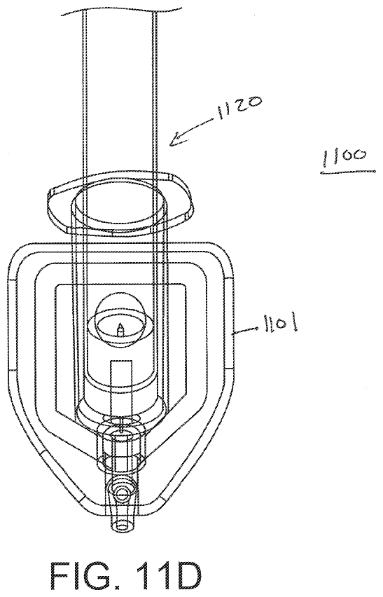

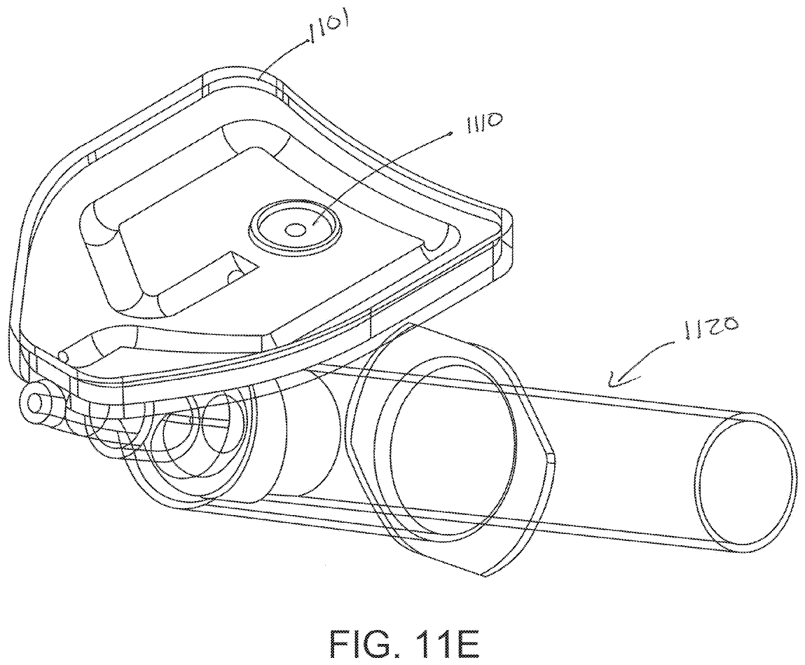

FIGS. 11A-11E illustrate a blood sequestration device 1100 in accordance with other alternative implementations. The blood sequestration device 1100 includes an inlet port 1102, similar to the inlet ports described above, a main outlet port 1104, and a sequestration channel port 1106 that splits off from the main outlet port 1104 and inlet port 1102. The sequestration channel port is connected to a sequestration chamber 1108. In the implementation shown in FIGS. 11A-11E, the blood sequestration device includes a base member 1101 having a channel therein, which functions as the sequestration chamber 1108. The channel can be formed as a tortuous path through the base member 1101, which is in turn shaped and formed to rest on a limb of a patient.

A portion of the sequestration chamber 1108 can protrude from the base member or near a top surface of the base member, just before exiting to an air permeable blood barrier 1110, to serve as a blood sequestration indicator 1109. The indicator 1109 can be formed of a clear material, or a material that changes color when in contact with blood.

In some implementations, the blood sequestration device 1100 can include a blood sampling device 1120 such as a normally closed needle, Vacutainer™ shield or other collection device. The blood sampling device 1120 can be manufactured and sold with the blood sequestration device 1100 for efficiency and convenience, so that a first aliquot of blood that may be contaminated by a patient needle insertion process can be sequestered. Thereafter, the blood sampling device 1120 can draw non-contaminated blood samples to reduce the risk of false positive testing and ensure a non-contaminated sample.

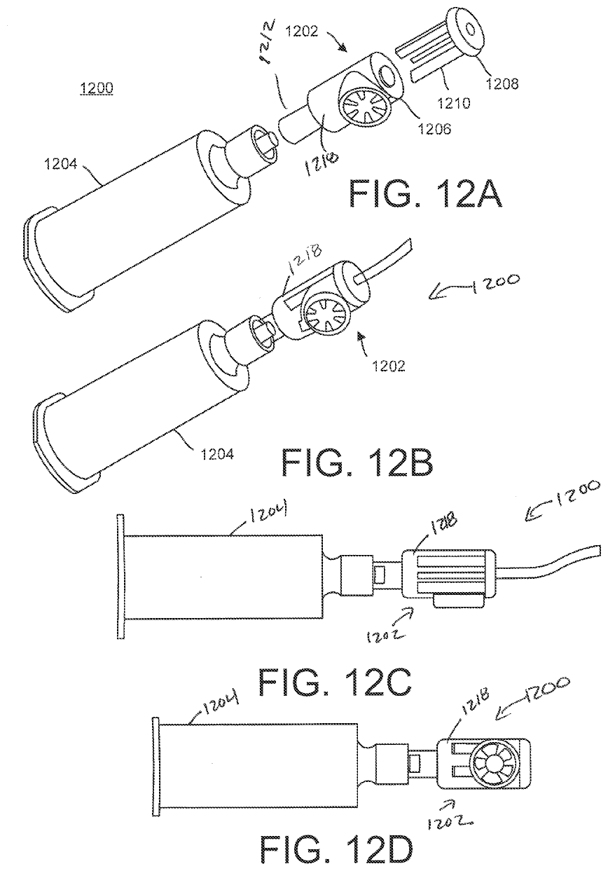

FIGS. 12A-12D illustrate a blood sample optimization system 1200 in accordance with yet other alternative implementations. The system 1200 includes a blood sequestration device 1202 for attaching to a blood sampling device 1204, such as a Vacutainer™ or other collection and sampling device. The blood sequestration device 1202 is configured and arranged to receive, prior to a Vacutainer™ container or vial being attached to a collection needle of the blood sampling device 1204, a first aliquot or amount of blood, and sequester that first aliquot or amount in a sequestration channel of the blood sequestration device 1202.

In some implementations, the blood sequestration device 1202 can include an inlet port 1212, a main outlet port, and a sequestration channel port. The inlet port 1212 can be connected to a patient needle or related tubing. The main outlet port 1214 can be connected to a normally closed needle or device to enable connection with an evacuated blood collection container or other collection device such as a Vacutainer™, associated tubing, luer connectors, syringe, a Luer activated valve, or the like. The sequestration channel port splits off from the main outlet port to a sequestration chamber 1218.

In some implementations, the sequestration chamber 1218 is formed as a channel within the body of a sequestration device 1202. The sequestration chamber 1218 can be a winding channel, such as a U-shaped channel, an S-shaped channel, a helical channel, or any other winding channel. The sequestration device 1202 can include a housing or other containing body, and one or more channels formed therein. As shown in FIGS. 12A and 12B, the sequestration device 1202 includes a main body 1206 and a cap 1208. The main body 1206 is formed with one or more cavities or channels, which are further formed with one or more arms 1210 that extend from the cap 1208, and which abut the cavities or channels in the main body 1206 to form the primary collection port and main outlet port.

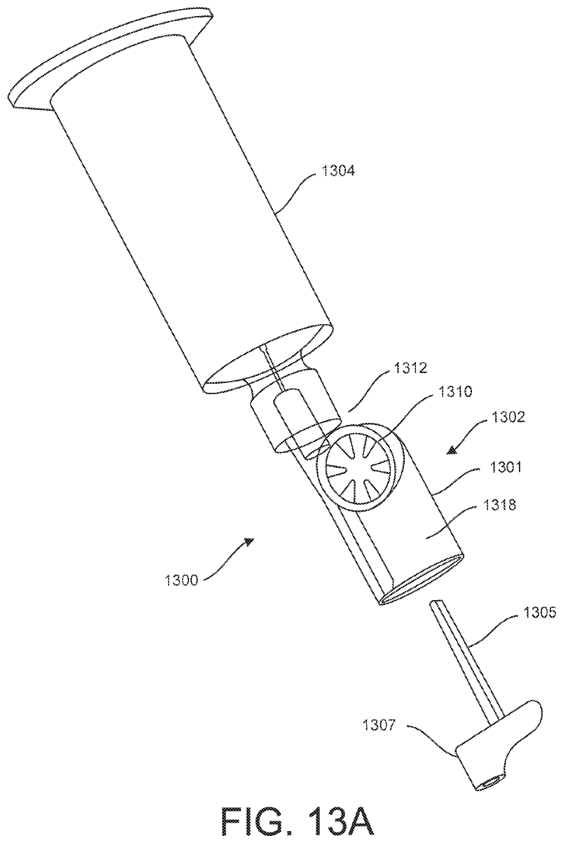

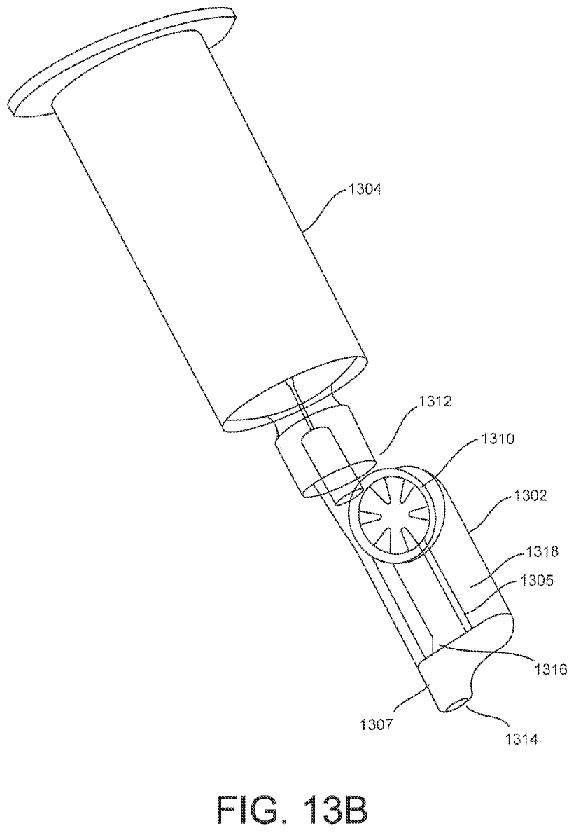

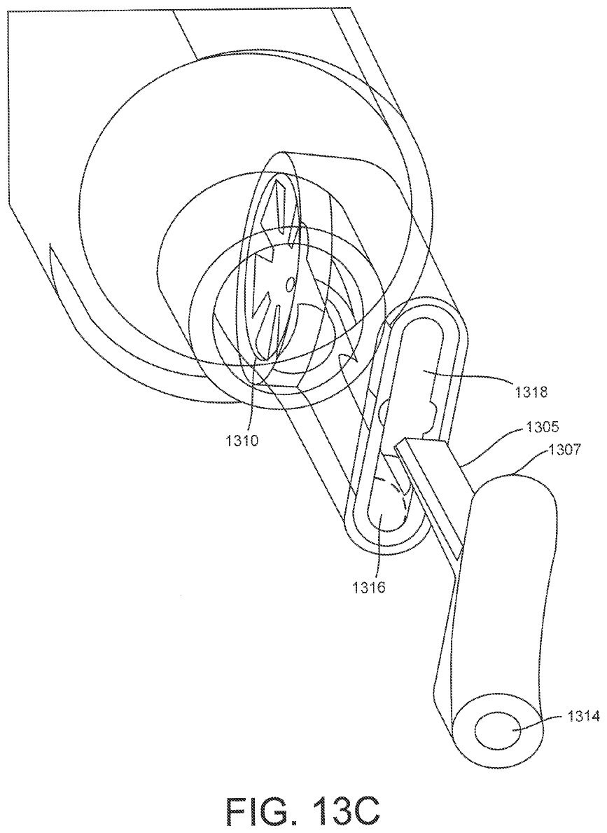

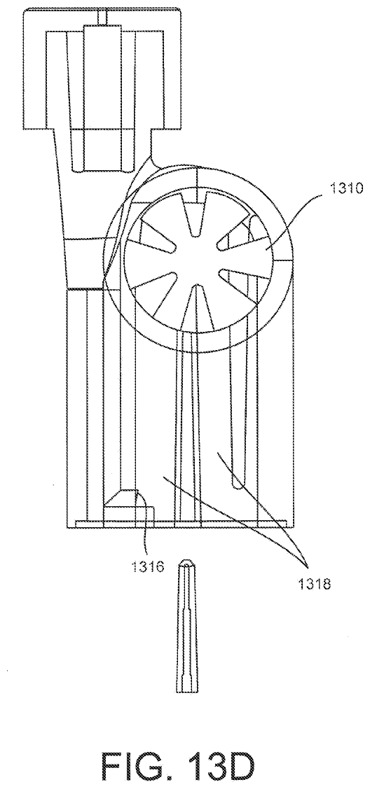

FIGS. 13A-13D illustrate a blood sample optimization system 1300 in accordance with yet other alternative implementations. The system 1300 includes a blood sequestration device 1302 for attaching to a blood sampling device 1304, such as a Vacutainer or other bodily fluid collection and sampling device. The blood sequestration device 1302 is configured and arranged to receive, prior to a Vacutainer container or vial being attached to a collection needle of the blood sampling device 1304, a first aliquot or amount of blood, and to sequester that first aliquot or amount of blood or other bodily fluid in a sequestration channel of the blood sequestration device 1302.

The blood sequestration device 1302 includes a housing 1301 having an inlet port 1314, a main outlet port 1312, and a sequestration channel port 1316. The inlet port 1314 can be connected to a patient needle or associated tubing. The main outlet port 1312 can be connected to a normally closed needle or device to enable connection with an evacuated blood collection container or other collection device such as a Vacutainer™ associated tubing, luer connectors, syringe, a Luer activated valve, or the like. The sequestration channel port 1316 splits off from the main inlet port 1314 to a sequestration chamber 1318.

In the implementation shown in FIGS. 13A-D, the sequestration chamber 1318 is formed as a cavity or chamber within housing 1301 or formed by walls that define housing 1301. The sequestration chamber 1318 can be a winding channel, such as a U-shaped channel, an S-shaped channel, a helical channel, or any other winding channel, that is defined by the cooperation and connection of housing 1301 with cap 1307 which cap 1307 can include a protrusion 1305 that provides one or more walls or directors for the winding channel in the sequestration chamber 1318. The protrusion 1305 from the cap 1307 can be straight or curved, and may have various channels, apertures or grooves embedded therein, and can extend from the cap 1307 any angle or orientation. When the cap 1307 is connected with the housing 1301 to complete the formation of the sequestration chamber 1318, the protrusion 1305 forms at least part of the winding channel to sequester a first aliquot or amount of blood or other bodily fluid in a sequestration channel formed in the sequestration chamber 1318 and by the winding channel.

The sequestration chamber 1318 includes an air permeable blood barrier 1310, substantially as described above. Air in the sequestration chamber 1318 is displaced through the air permeable blood barrier 1310 by an initial aliquot of blood that is provided into the sequestration chamber 1318 by the blood pressure of the patient. Once the sequestration chamber 1318 is filled and the air in the sequestration chamber 1318 displaced, the blood pressure of the patient will be insufficient to drive or provide further blood into the blood sequestration device 1302, and in particular the outlet port 1312, until a force such as a vacuum or other pressure, such as provided by the blood sample collection device like Vacutainer is provided to draw out a next aliquot or amount of blood or bodily fluid. Further blood draws through the main outlet port 1312 can be accomplished, where these samples will be non-contaminated since any contaminants would be sequestered in the sequestration chamber 1318 with the first aliquot of blood.

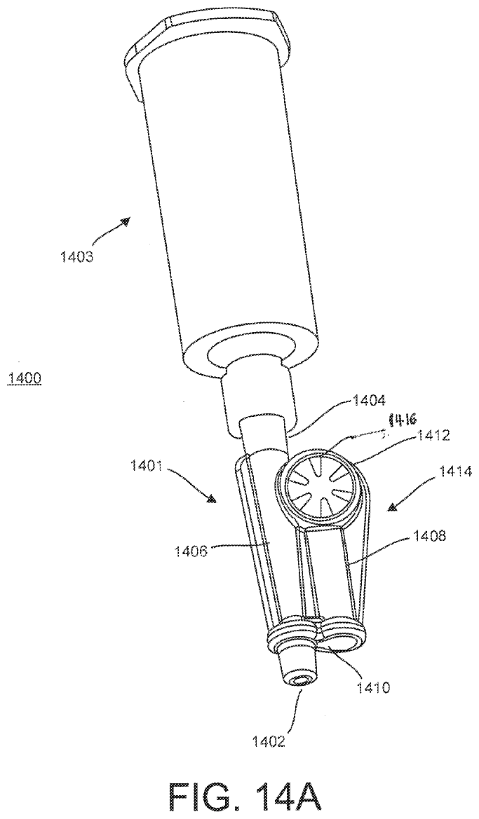

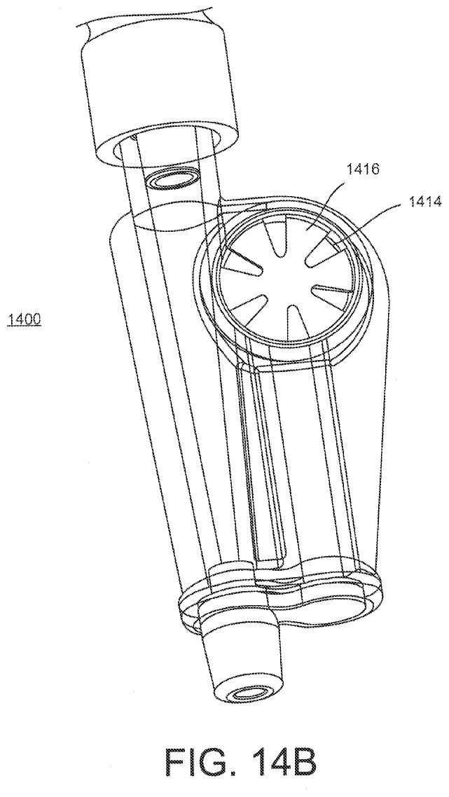

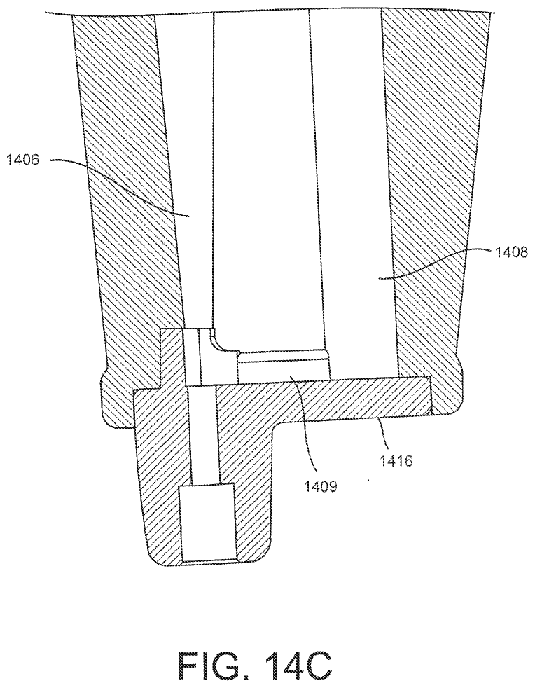

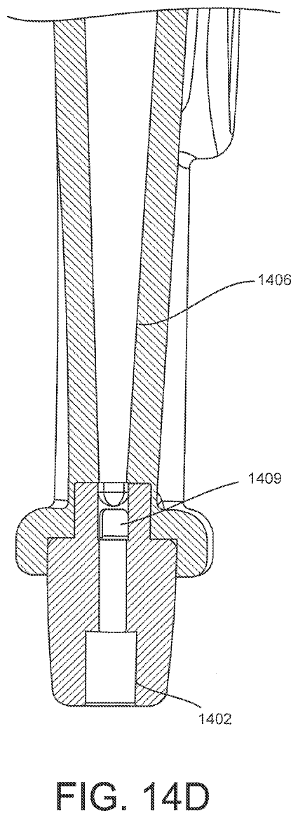

FIGS. 14A-14E illustrate yet another implementation of a blood sampling system 1400 to sequester contaminates of an initial aliquot or sample to reduce false positives in blood cultures or tests performed on a patient's blood sample. The blood sampling system 1400 includes a blood sequestration device 1401 that can be connected between a blood sample collection device 1403 and a patient needle (not shown). The blood sample collection device 1403 can be a Vacutainer or the like. The blood sequestration device 1401 includes an inlet port 1402 that can be connected with a patient needle that is inserted into a patient's vascular system for access to and withdrawing of a blood sample. The inlet port 1402 may also be connected with tubing or other conduit that is in turn connected with the patient needle.

The inlet port 1402 defines an opening into the blood sequestration device 1401, which opening can be the same cross sectional dimensions as tubing or other conduit connected with the patient needle or the patient needle itself. For instance, the opening can be circular with a diameter of approximately 0.045 inches, but can have a diameter of between 0.01 inches or less to 0.2 inches or more. The blood sequestration device 1401 further includes an outlet port 1404, which defines an opening out of the blood sequestration device 1401 and to the blood sample collection device 1403. The outlet port 1404 may also be connected with tubing or other conduit that is in turn connected with the blood sequestration device 1403. The outlet port 1404 can further include a connector device such as a threaded cap, a Luer connector (male or female), a non threaded interference or glue joint fitting for attachment of various devices including but not limited to tubing, or the like.

The blood sequestration device 1401 further includes a sampling channel 1406 between the inlet port 1402 and the outlet port 1404, and which functions as a blood sample pathway once a first aliquot of blood has been sequestered. The sampling channel 1406 can be any sized, shaped or configured channel, or conduit. In some implementations, the sampling channel 1406 has a substantially similar cross sectional area as the opening of the inlet port 1402. In other implementations, the sampling channel 1406 can gradually widen from the inlet port 1402 to the outlet port 1404.

The blood sequestration device 1401 further includes a sequestration chamber 1408 that is connected to and split off or diverted from the sampling channel 1406 at any point between the inlet port 1402 and the outlet port 1404, but preferably from a proximal end of the sampling channel 1406 near the inlet port 1402. The sequestration chamber 1408 is at first maintained at atmospheric pressure, and includes an air outlet 1412 at or near a distal end of the sequestration chamber 1408 opposite the diversion point from the sampling channel 1406. The air outlet 1412 includes an air permeable blood barrier 1412. As shown in FIG. 14B, the air permeable blood barrier 1412 can be overlaid with a protective cover 1416. The protective cover 1416 can be sized and configured to inhibit a user from touching the air permeable blood barrier 1412 with their finger or other external implement, while still allowing air to exit the air permeable blood barrier 1412 as the air is displaced from the sequestration chamber 1408 by blood being forced into the sequestration chamber 1408 by a patient's own blood pressure. In addition the protective cover 1416 can be constructed to inhibit or prevent accidental exposure of the air permeable blood barrier to environmental fluids or splashes. This can be accomplished in a variety of mechanical ways including but not limited to the addition of a hydrophobic membrane to the protective cover.

As shown in FIGS. 14C and 14D, the sampling channel 1406 can be cylindrical or frusto-conical in shape, going from a smaller diameter to a larger diameter, to minimize a potential to lyse red blood cells. Likewise, the sampling channel 1406 is formed with a minimal amount of or no sharp turns or edges, which can also lyse red blood cells. The sampling channel 1406 splits off to the sequestration chamber 1408 near the inlet port 1402 via a diversion pathway 1409. The diversion pathway 1409 can have any cross-sectional shape or size, but is preferably similar to the cross-sectional shape of at least part of the inlet port 1402.

In some implementations, the sampling channel 1406 and the sequestration chamber 1408 are formed by grooves, channels, locks or other pathways formed in housing 1414. The housing 1414 can be made of plastic, metal or other rigid or semi-rigid material. The housing 1414 can have a bottom member that sealably mates with a top member. One or both of the bottom member and the top member can include the sampling channel 1406 and the sequestration chamber 1408, as well as the diversion pathway 1409, the inlet port 1402, and the outlet port 1404. In some other implementations, one or more of the diversion pathway 1409, the inlet port 1402, and/or the outlet port 1404 can be at least partially formed by a cap member that is connected to either end of the housing 1414. In some implementations, the top member and the bottom member, as well as the cap member(s), can be coupled together by laser welding, heat sealing, gluing, snapping, screwing, bolting, or the like. In other implementations, some or all of the interior surface of the diversion pathway 1409 and/or sequestration chamber 1408 can be coated or loaded with an agent or substance, such as a decontaminate, solidifying agent, or the like. For instance, a solidifying agent can be provided at the diversion pathway 1409 such that when the sequestration chamber 1408 is filled and the initial aliquot of blood backs up to the diversion pathway 1409, that last amount of sequestered blood could solidify, creating a barrier between the sequestration chamber 1408 and the sampling channel 1406.

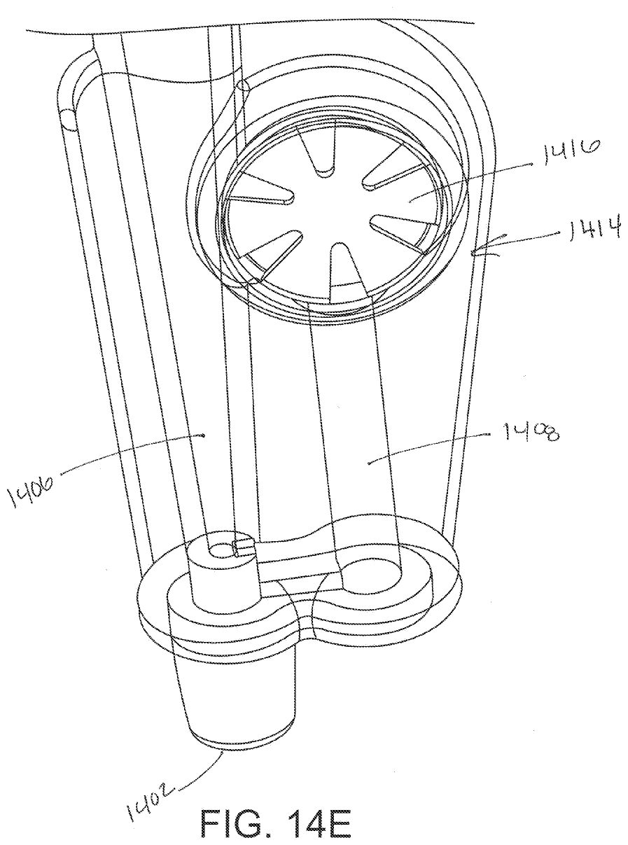

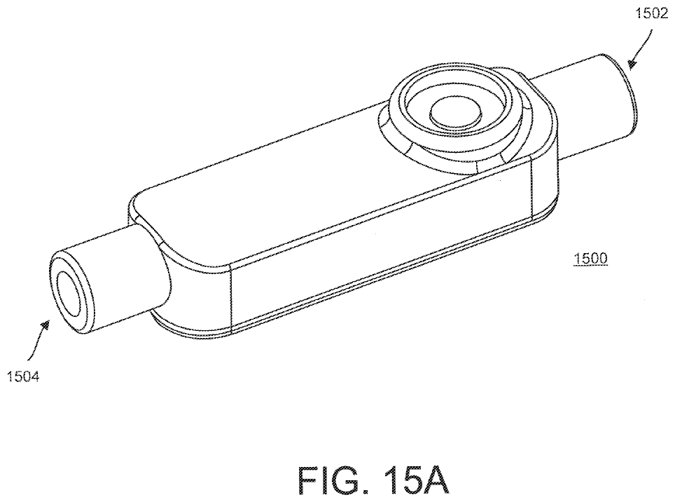

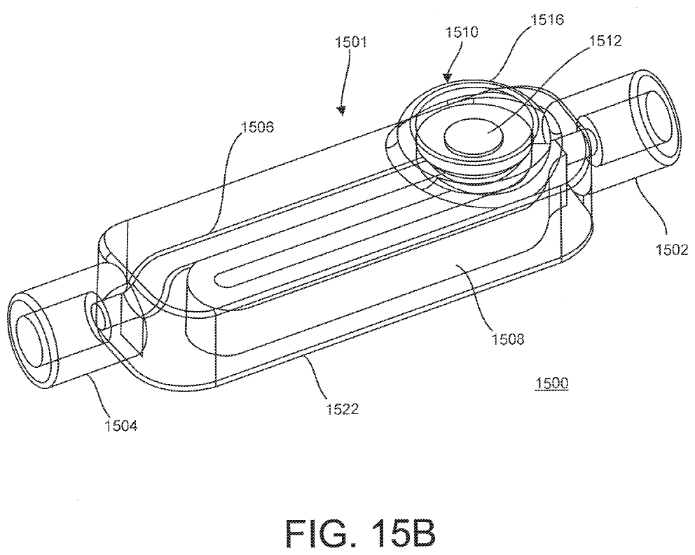

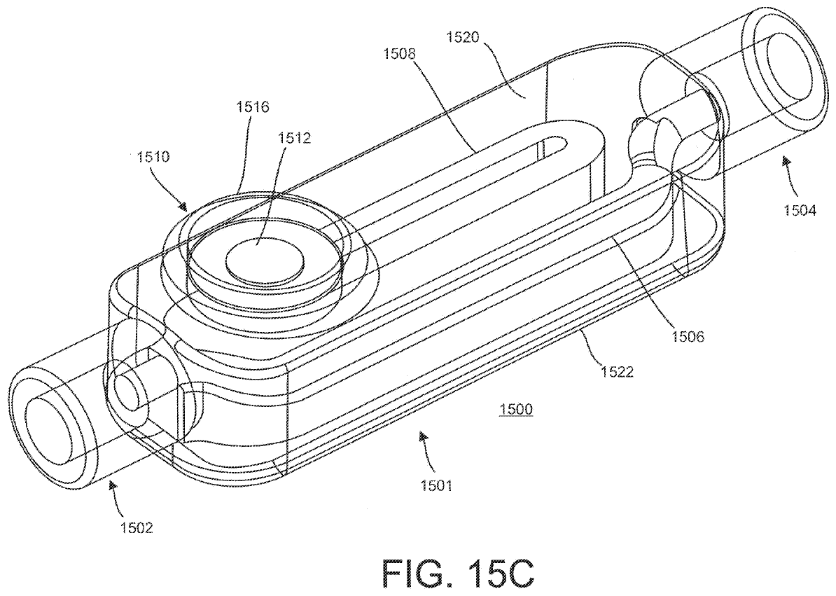

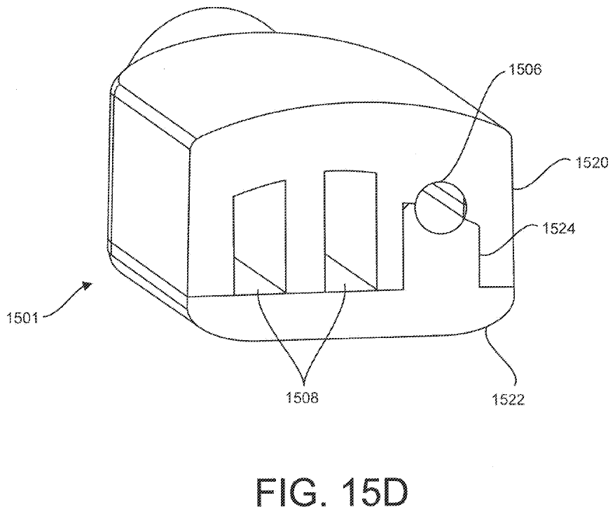

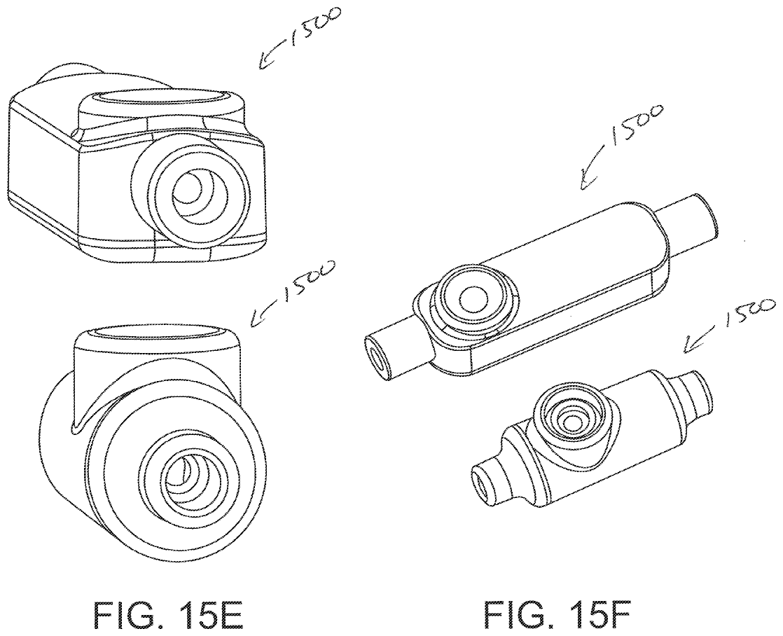

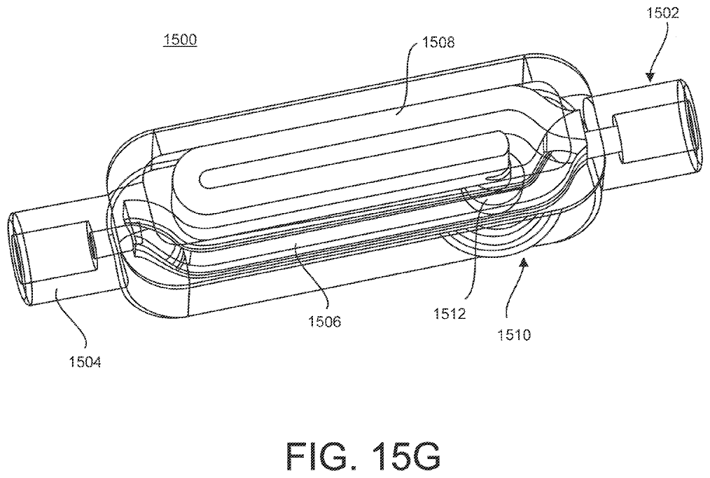

FIGS. 15A-15G illustrate a blood sequestration device 1500. The blood sequestration device 1500 can be connected to a normally closed needle or device to enable connection with an evacuated blood collection container or other collection device such as a Vacutainer™, associated tubing, luer connectors, syringe, a Luer activated valve, or the like.

The blood sequestration device 1500 includes an inlet port 1502 that can be connected with a patient needle that is inserted into a patient's vascular system for access to and withdrawing of a blood sample. The inlet port 1502 may also be connected with tubing or other conduit that is in turn connected with the patient needle. The inlet port 1502 defines an opening into the blood sequestration device 1500, which opening may be the same cross sectional dimensions as tubing or other conduit connected with the patient needle or the patient needle itself. For instance, the opening can be circular with a diameter of approximately 0.045 inches, but can have a diameter of between 0.01 inches or less to 0.2 inches or more.

The inlet port 1502 can also include a sealing or fluid-tight connector or connection, such as threading or Luer fitting, or the like. In some implementations, tubing or other conduit associated with the patient needle can be integral with the inlet port 1502, such as by co-molding, gluing, laser weld, or thermally bonding the parts together. In this manner, the blood sequestration device 1500 can be fabricated and sold with the patient needle as a single unit, eliminating the need for connecting the patient needle to the blood sequestration device 1500 at the time of blood draw or sampling.

The blood sequestration device 1500 further includes an outlet port 1504, which defines an opening out of the blood sequestration device 1500 and to the blood sample collection device. The outlet port 1504 may also be connected with tubing or other conduit that is in turn connected with the blood sequestration device, and may also include a sealing or fluid-tight connector or connection, such as threading or Luer fitting, or the like. Accordingly, as discussed above, the blood sequestration device 1500 can be fabricated and sold with the patient needle and/or tubing and the blood sample collection device as a single unit, eliminating the need for connecting the patient needle and the blood sample collection device to the blood sequestration device 1500 at the time of blood draw or sampling.

The blood sequestration device 1500 further includes a sampling channel 1506 between the inlet port 1502 and the outlet port 1504, and which functions as a blood sample pathway once a first aliquot of blood has been sequestered. The sampling channel 1506 can be any sized, shaped or configured channel or conduit. In some implementations, the sampling channel 1506 has a substantially similar cross sectional area as the opening of the inlet port 1502. In other implementations, the sampling channel 1506 can gradually widen from the inlet port 1502 to the outlet port 1504.

The blood sequestration device 1500 further includes a sequestration chamber 1508 that is connected to and split off or diverted from the sampling channel 1506 at any point between the inlet port 1502 and the outlet port 1504, but preferably from a proximal end of the sampling channel 1506 near the inlet port 1502. In some implementations, the diversion includes a Y-shaped junction. The sequestration chamber 1508 is preferably maintained at atmospheric pressure, and includes a vent 1510 at or near a distal end of the sequestration chamber 1508. The vent 1510 includes an air permeable blood barrier 1512. FIG. 15C illustrates the blood sequestration device 1500 with the sequestration chamber 1508 filled with a first aliquot or sample of blood from the patient.

The air permeable blood barrier 1512 can be covered with a protective cover 1516. The protective cover 1516 can be sized and configured to inhibit a user from touching the air permeable blood barrier 1512 with their finger or other external implement, while still allowing air to exit the air permeable blood barrier 1512 as the air is displaced from the sequestration chamber 1508 by blood being forced into the sequestration chamber 1508 by a patient's own blood pressure. The protective cover 1516 can be constructed to inhibit or prevent accidental exposure of the filter to environmental fluids or splashes. This can be accomplished in a variety of mechanical ways including but not limited to the addition of a hydrophobic membrane to the protective cover.

FIG. 15B is a perspective view of the blood sequestration device 1500 from the outlet port 1504 and top side of a housing 1501 of the blood sequestration device 1500 that includes the vent 1510, and illustrating an initial aliquot of blood filling sequestration chamber 1508 while the sampling channel 1506 is empty, before a sample collection device is activated. FIG. 15G is a perspective view of the blood sequestration device 1500 from the outlet port 1504 and bottom side of the housing 1501 of the blood sequestration device 1500, and illustrating the initial aliquot of blood filling sequestration chamber 1508 while the sampling channel 1506 is empty, before the sample collection device is activated. FIG. 15C is another perspective view of the blood sequestration device 1500 from the inlet port 1502 and top side of a housing 1501 of the blood sequestration device 1500 that includes the vent 1510, and illustrating blood now being drawn through sampling channel 1506 while the sequestered blood remains substantially in the sequestration chamber 1508.

FIG. 15D is a cross section of the blood sequestration device 1500 in accordance with some implementations, showing the housing 1501 that defines the sampling channel 1506 and the sequestration chamber 1508. FIGS. 15E and 15F illustrate various form factors of a housing for a blood sequestration device, in accordance with one or more implementations described herein.

The sequestration chamber 1508 can have a larger cross-sectional area than the sampling channel 1506, and the cross-sectional area and length can be configured for a predetermined or specific volume of blood to be sequestered or locked. The sampling channel 1506 can be sized to be compatible with tubing for either or both of the patient needle tubing or the blood collection device tubing.

The housing 1501 can be formed of multiple parts or a single, unitary part. In some implementations, and as illustrated in FIG. 15D, the housing 1501 includes a top member 1520 and a bottom member 1522 that are mated together, one or both of which having grooves, channels, locks, conduits or other pathways pre-formed therein, such as by an injection molding process or by etching, cutting, drilling, etc. The top member 1520 can be connected with the bottom member 1522 by any mating or connection mechanism, such as by laser welding, thermal bonding, ultrasonic welding, gluing, using screws, rivets, bolts, or the like, or by other mating mechanisms such as latches, grooves, tongues, pins, flanges, or the like.

In some implementations, such as shown in FIG. 15D, the top member 1520 can include the grooves, channels, locks, conduits or other pathways, while the bottom member 1522 can include a protrusion 1524 that is sized and adapted to fit into at least one of the grooves, channels, locks or other pathways of the top member 1520. The protrusion 1524 can provide a surface feature, such as a partial groove or channel, for instance, to complete the formation of either the sampling channel 1506 and/or the sequestration chamber 1508. In some implementations, the protrusion 1524 can be formed with one or more angled sides or surfaces for a tighter fit within the corresponding groove, channel, lock or other pathway. In yet other implementations, both the top member 1520 and the bottom member can include grooves, channels, locks or other pathways, as well as one or more protrusions 1524.

In some implementations, the sampling channel 1506 and the sequestration chamber 1508 are formed by grooves, channels, locks or other pathways formed in housing 1501. The housing 1501 can be made of any suitable material, including rubber, plastic, metal or other material. The housing 1501 can be formed of a clear or translucent material, or of an opaque or non-translucent material. In other implementations, the housing 1501 can be mostly opaque or non-translucent, while the housing surface directly adjacent to the sampling channel 1506 and/or the sequestration chamber 1508 is clear or translucent, giving a practitioner a visual cue or sign that the sequestration chamber 1508 is first filled to the extent necessary or desired, and/or then a visual cue or sign that the sequestered blood remains sequestered while a clean sample of blood is drawn through the sampling channel 1506. Other visual cues or signs of the sequestration can include, without limitation: the air permeable blood barrier 1512 turning a different color upon contact, saturation, or partial saturation with blood; a color-coded tab or indicator at any point along or adjacent to the sequestration chamber; an audible signal; a vibratory signal; or other signal.

After a venipuncture by a patient needle of a patient (not shown), which could gather a number of pathogens from the patient's skin, a first amount of the patient's blood with those pathogens will make its way into the inlet port 1502 blood sequestration device 1500 and flow into the sequestration chamber 1508 by following the path of least resistance, as the patient's own blood pressure overcomes the atmospheric pressure in the sequestration chamber 1508 to displace air therein through the air permeable blood barrier 1512. The patient's blood pressure will not be sufficient to overcome the air pressure that builds up in the sealed sampling channel 1506. Eventually, the sequestration chamber 1508, which has a predetermined volume, is filled with blood that displaces air through the air permeable blood barrier 1512. Once the blood hits the air permeable blood barrier, the blood interacts with the air permeable blood barrier 1512 material to completely or partially seal the vent 1510. A signal or indication may be provided that the practitioner can now utilize the Vacutainer capsule or other blood sample collection device to acquire a next amount of the patient's blood for sampling. The blood in the sequestration chamber 1508 is now effectively sequestered in the sequestration chamber.

Upon filling the blood sequestration pathway 1508 but prior to use of the Vacutainer or other blood sample collection device, the patient's blood pressure may drive compression of the air in the sampling channel 1506, possibly resulting in a small amount of blood moving past the diversion point to the sequestration chamber 1508 and into the sampling channel 1506, queuing up the uncontaminated blood to be drawn through the sampling channel 1506.

In some instances, as shown in FIG. 15H, an inlet port 1532 can include a male luer connector for connecting to a removable patient needle, and an outlet port 11534 can include a female luer connector for connecting with a syringe. This implementation of the inlet port and outlet port can be used with any device described herein, for avoiding a propensity of a Vacutainer-type device collapsing a patient's vein. In this implementation, a clinician can use the syringe in a modulated fashion to obtain a blood sample. In operation, the syringe is attached to the outlet port 1004, and the needle is attached to the inlet port 1002. A venipuncture is performed with the needle, and without the clinician pulling on the syringe. An initial aliquot of blood fills a sequestration chamber, and then the syringe can be used to draw a sample of blood through the collection channel, bypassing the sequestered blood in the sequestration chamber.

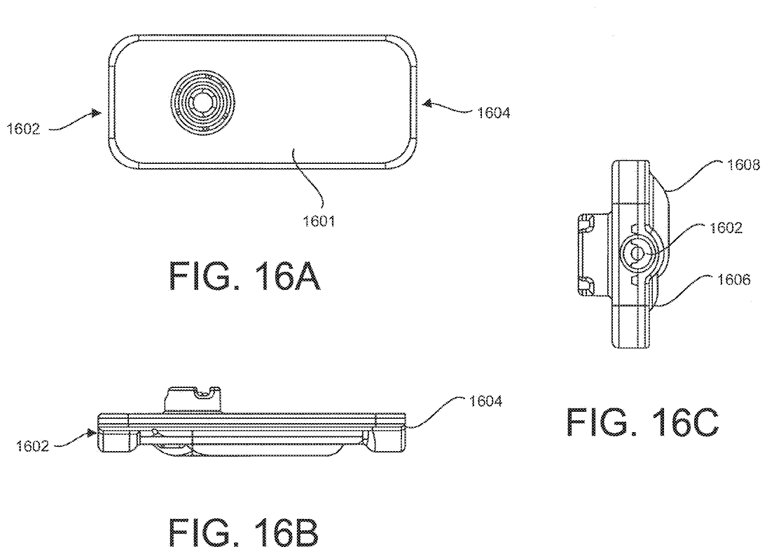

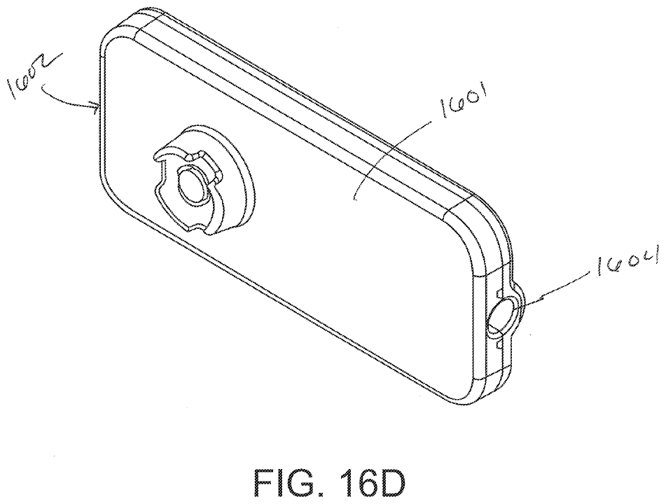

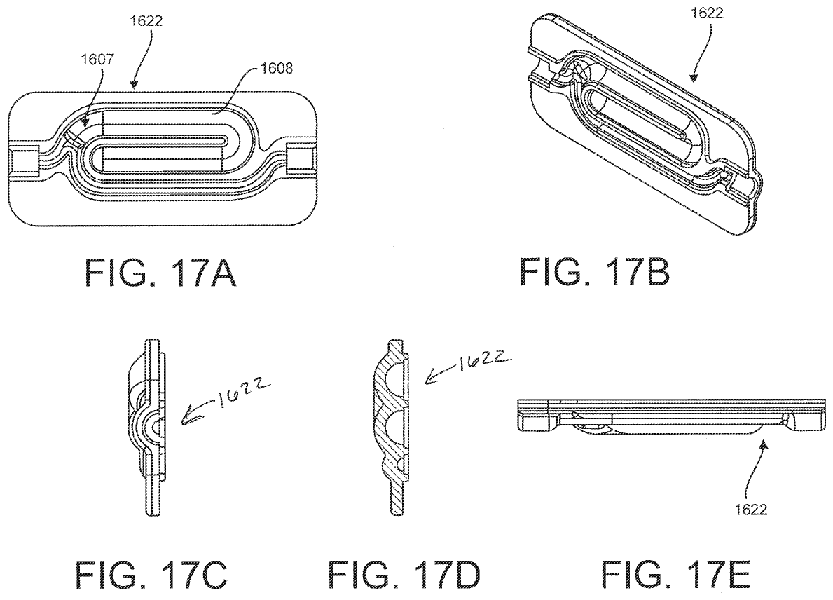

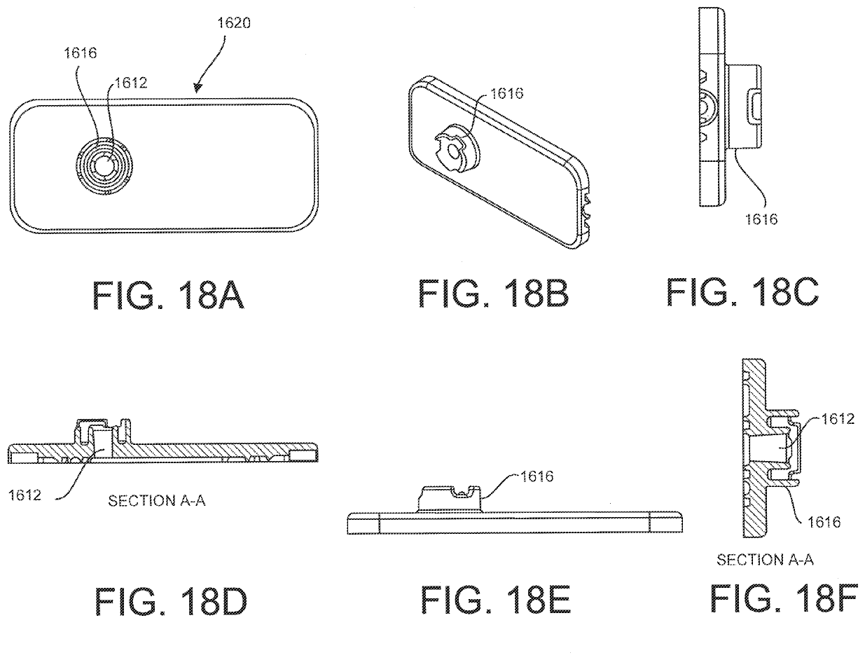

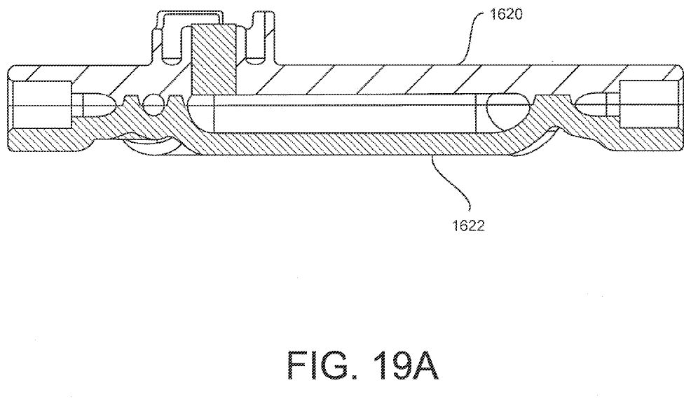

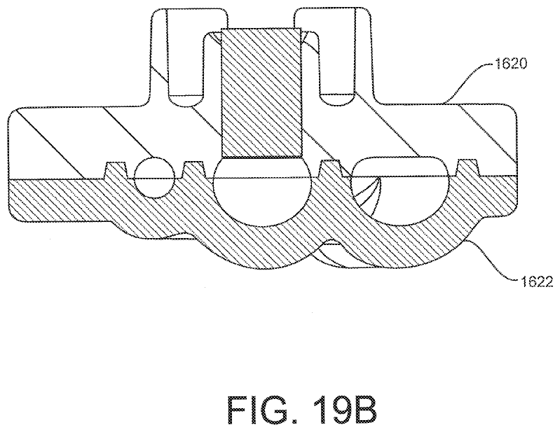

FIGS. 16-19 illustrate yet another implementation of a blood sequestration device. FIGS. 16A-16D illustrate a blood sequestration device 1600 that can be connected between a blood sample collection device, such as an evacuated blood collection container like a Vacutainer™ (not shown), and a patient needle (not shown) and/or associated tubing. FIG. 17 illustrates a bottom member of the blood sequestration device, and FIG. 18 illustrates a top member of the blood sequestration device, which top member and bottom member can be mated together to form an inlet port, and outlet port, a sequestration chamber and a sampling channel, as explained more fully below. FIGS. 19A and B show the top member and bottom member mated together. It should be understood that FIGS. 16-19 illustrate one exemplary manner of constructing a blood sequestration device as described herein, and other forms of construction are possible.

Referring to FIGS. 16A-D, the blood sequestration device 1600 includes an inlet port 1602 that can be connected with a patient needle that is inserted into a patient's vascular system for access to and withdrawing of a blood sample. The inlet port 1602 may also be connected with tubing or other conduit that is in turn connected with the patient needle. The inlet port 1602 defines an opening into the blood sequestration device 1600, which opening can be the same cross sectional dimensions as tubing or other conduit connected with the patient needle or the patient needle itself. For instance, the opening can be circular with a diameter of approximately 0.045 inches, but can have a diameter of between 0.01 inches or less to 0.2 inches or more.

The inlet port 1602 can also include a sealing or fluid-tight connector or connection, such as threading or Luer fitting, or the like. In some implementations, tubing or other conduit associated with the patient needle can be integral with the inlet port 1602, such as by co-molding, gluing, laser weld, or thermally bonding the parts together. In this manner, the blood sequestration device 1600 can be fabricated and sold with the patient needle and/or tubing as a single unit, eliminating the need for connecting the patient needle to the blood sequestration device 1600 at the time of blood draw or sampling.

The blood sequestration device 1600 further includes an outlet port 1604, which defines an opening out of the blood sequestration device 1600 and to the blood sample collection device. The outlet port 1604 may also be connected with tubing or other conduit that is in turn connected with the blood sequestration device, and may also include a sealing or fluid-tight connector or connection, such as threading or Luer fitting, or the like. Accordingly, as discussed above, the blood sequestration device 1600 can be fabricated and sold with the patient needle and/or tubing and the blood sample collection device as a single unit, eliminating the need for connecting the patient needle and the blood sample collection device to the blood sequestration device 1600 at the time of blood draw or sampling.

The blood sequestration device 1600 further includes a sampling channel 1606 between the inlet port 1602 and the outlet port 1604, and a sequestration chamber 1608 that is connected to and split off or diverted from the sampling channel 1606 at any point between the inlet port 1602 and the outlet port 1604. The sampling channel 1606 functions as a blood sampling pathway once a first aliquot of blood has been sequestered in the sequestration chamber 1608. The sampling channel 1606 can be any sized, shaped or configured channel, or conduit. In some implementations, the sampling channel 1606 has a substantially similar cross sectional area as the opening of the inlet port 1602. In other implementations, the sampling channel 1606 can gradually widen from the inlet port 1602 to the outlet port 1604. The sequestration chamber 1608 may have a larger cross section to form a big reservoir toward the sequestration channel path so that the blood will want to enter the reservoir first versus entering a smaller diameter on the sampling channel 1606, as is shown more fully in FIGS. 17 and 19.

In some exemplary implementations, the diversion between the sampling channel 1606 and the sequestration chamber 1608 is by diverter junction 1607. Diverter junction 1607 may be a substantially Y-shaped, T-shaped, or U-shaped. In some preferred exemplary implementations, and as shown in FIG. 17A-17B, the diverter junction 1607 is configured such that the flow out of the inlet port 1602 is preferentially directed toward the sequestration chamber 1608. The sequestration chamber 1608 may also include or form a curve or ramp to direct the initial blood flow toward and into the sequestration chamber 1608.

The sequestration chamber 1608 is preferably maintained at atmospheric pressure, and includes a vent 1610 at or near a distal end of the sequestration chamber 1608. The vent 1610 may include an air permeable blood barrier 1612 as described above.

The blood sequestration device 1600 can include a housing 1601 that can be formed of multiple parts or a single, unitary part. In some implementations, and as illustrated in FIGS. 17A-17E and FIGS. 18A-18F, the housing 1601 includes a top member 1620 and a bottom member 1622 that are mated together. The blood sequestration device 1600 can also include a gasket or other sealing member (not shown) so that when the top member 1620 is mechanically attached with the bottom member 1622, the interface between the two is sealed by the gasket or sealing member. The FIGS. 17A-17E illustrate a bottom member 1622 of a housing for a blood sequestration device 1600. The bottom member 1622 can include grooves, channels, locks, conduits or other pathways pre-formed therein, such as by an injection molding process or by etching, cutting, drilling, etc., to form the sampling channel 1606, the sequestration chamber 1608, and diverter junction 1607.

The sequestration chamber 1608 may have a larger cross section than the sampling channel 1606 so that the blood will preferentially move into the sequestration chamber first versus entering a smaller diameter on the sampling channel 1606.

FIGS. 18A-18F illustrate the top member 1620, which can be connected with the bottom member 1622 by any mating or connection mechanism, such as by laser welding, thermal bonding, gluing, using screws, rivets, bolts, or the like, or by other mating mechanisms such as latches, grooves, tongues, pins, flanges, or the like. The top member 1620 can include some or all of the grooves, channels, locks, conduits or other pathways to form the sampling channel 1606, the sequestration chamber 1608, and the diverter junction 1607. In yet other implementations, both the top member 1620 and the bottom member 1622 can include the grooves, channels, locks or other pathways.

In some implementations, the sampling channel 1606 and the sequestration chamber 1608 are formed by grooves, channels, locks or other pathways formed in housing 1601. The housing 1601 can be made of rubber, plastic, metal or any other suitable material. The housing 1601 can be formed of a clear or translucent material, or of an opaque or non-translucent material. In other implementations, the housing 1601 can be mostly opaque or non-translucent, while the housing surface directly adjacent to the sampling channel 1606 and/or the sequestration chamber 1608 may be clear or translucent, giving a practitioner a visual cue or sign that the sequestration chamber 1608 is first filled to the extent necessary or desired, and/or then a visual cue or sign that the sequestered blood remains sequestered while a clean sample of blood is drawn through the sampling channel 1606. Other visual cues or signs of the sequestration can include, without limitation: the air permeable blood barrier 1612 turning a different color upon contact, saturation, or partial saturation with blood; a color-coded tab or indicator at any point along or adjacent to the sequestration chamber; an audible signal; a vibratory signal; or other signal.

As shown in FIGS. 18A-18F, the air permeable blood barrier 1612 can be covered with, or surrounded by, a protective member 1616. The protective member 1616 can be sized and configured to inhibit a user from touching the air permeable blood barrier 1612 with their finger or other external implement, while still allowing air to exit the air permeable blood barrier 1612 as the air is displaced from the sequestration chamber 1608. In some implementations, the protective member 1616 includes a protrusion that extends up from a top surface of the top member 1620 and around the air permeable blood barrier 1612. The protective member 1616 can be constructed to inhibit or prevent accidental exposure of the filter to environmental fluids or splashes. This can be accomplished in a variety of mechanical ways including but not limited to the addition of a hydrophobic membrane to the protective cover.

In use, the blood sequestration device 1600 includes a sampling channel 1606 and a sequestration chamber 1608. Both pathways are initially air-filled at atmospheric pressure, but the sampling channel 1606 is directed to an outlet port 1604 that will be initially sealed by a Vacutainer or other such sealed blood sampling device, and the sequestration chamber 1608 terminates at a vent 1610 to atmosphere that includes an air permeable blood barrier 1612.

After a venipuncture by a patient needle of a patient (not shown), which could gather a number of pathogens from the patient's skin, a first amount of the patient's blood with those pathogens will pass through inlet port 1602 of blood sequestration device 1600. This initial volume of potentially contaminated blood will preferentially flow into the sequestration chamber 1608 by finding the path of least resistance. The patient's own blood pressure overcomes the atmospheric pressure in the vented sequestration chamber 1608 to displace air therein through the air permeable blood barrier 1612, but is not sufficient to overcome the air pressure that builds up in the sealed sampling channel 1606. In various exemplary embodiments, the sequestration chamber 1608 and sampling channel 1606 can be configured such that the force generated by the patient's blood pressure is sufficient to overcome any effect of gravity, regardless of the blood sequestration device's orientation.

Eventually, the sequestration chamber 1608 fills with blood that displaces air through the air permeable blood barrier 1612. Once the blood contacts the air permeable blood barrier, the blood interacts with the air permeable blood barrier 1612 material to completely or partially seal the vent 1610. A signal or indication may be provided that the practitioner can now utilize the Vacutainer or other blood sampling device.

Upon filling the blood sequestration pathway 1608 but prior to use of the Vacutainer or other blood sample collection device, the patient's blood pressure may drive compression of the air in the sampling channel 1606, possibly resulting in a small amount of blood moving past the diversion point into the sampling channel 1606, queuing up the uncontaminated blood to be drawn through the sampling channel 1606.

FIG. 19A is a side view, and FIG. 19B is a cross-sectional view, of the blood sequestration device 1600, illustrating the top member 1620 mated with the bottom member 1622.

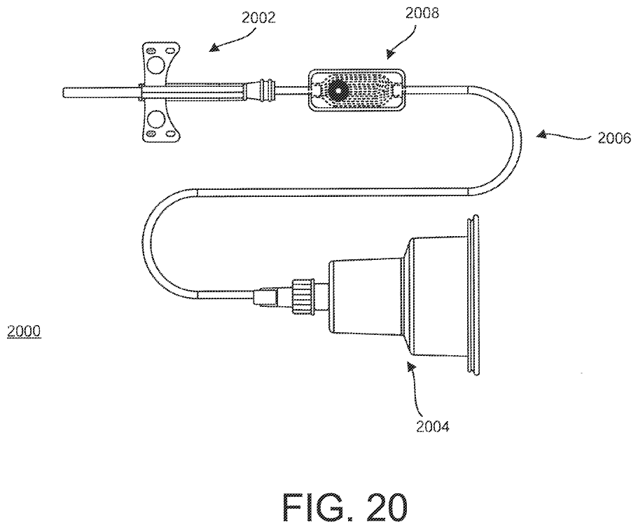

FIG. 20 shows a blood sample optimization system 2000 that includes a patient needle 2002 for vascular access to a patient's bloodstream, a blood sample collection device 2004 to facilitate the collecting of one or more blood samples, and a conduit 2006 providing a fluid connection between the patient needle 2002 and the blood sample collection device 2004. In some implementations, the blood sample collection device 2004 includes a protective shield that includes a sealed collection needle on which a sealed vacuum-loaded container is placed, which, once pierced by the collection needle, draws in a blood sample under vacuum pressure or force through the conduit 2006 from the patient needle 2002.

The blood sample optimization system 2000 further includes a blood sequestration device 2008, located at any point on the conduit 2006 between the patient needle 2002 and the blood sample collection device 2004 as described herein.

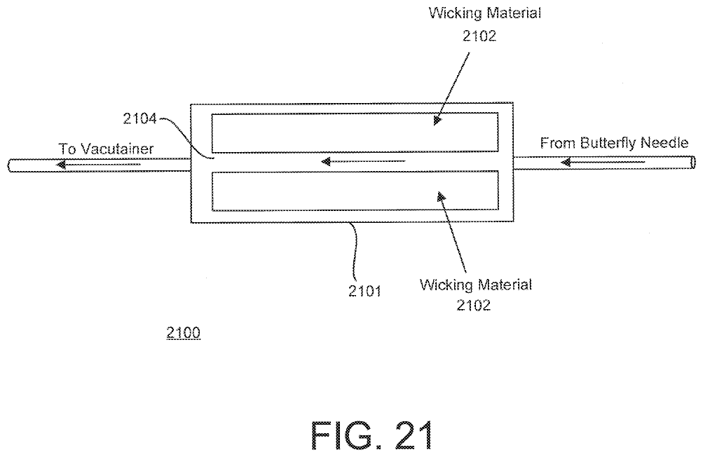

FIG. 21 illustrates a non-vented blood sequestration device 2100 using a wicking material chamber. The blood sequestration device 2100 includes a housing 2101 that has a sampling channel 2104 that is at least partially surrounded or abutted by a sequestration chamber 2102 that is filled with a wicking material. An initial aliquot of blood is drawn in from the patient needle into the sampling channel 2104 where it is immediately wicked into the wicking material of the sequestration chamber 2102. The wicking material and/or sequestration chamber 2102 is sized and adapted to receive and hold a predetermined amount of blood, such that follow-on or later blood draws pass by the wicking material and flow straight through the sampling channel 2104 to a sampling device such as a Vacutainer. The wicking material can include a substance such as a solidifier, a decontaminate, or other additive.

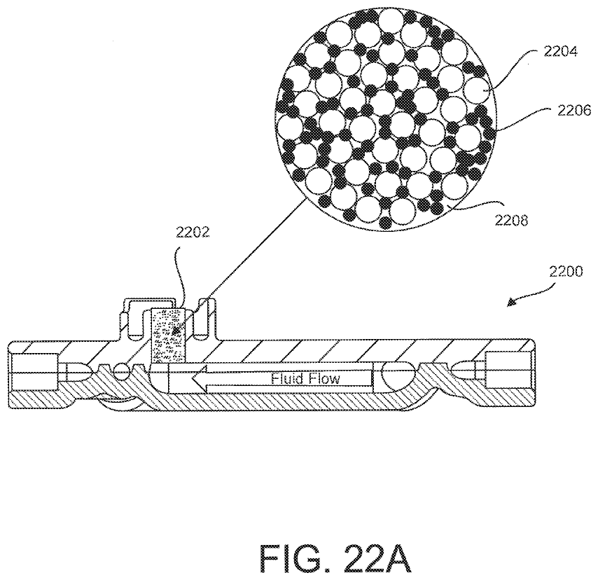

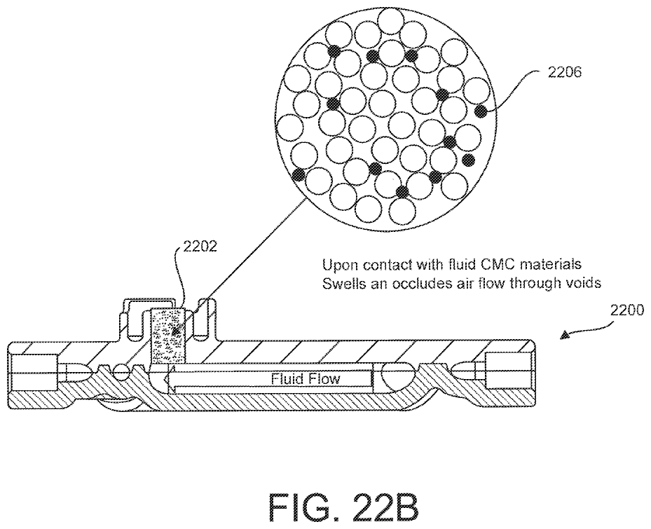

As described herein, an air permeable blood barrier may be created using a wide variety of different structures and materials. As shown in FIGS. 22A and 22B, an air permeable blood barrier 2202 of a blood sequestration device 2200 can include a polymer bead matrix 2204, in which at least some beads are treated to make them hydrophilic. The air permeable blood barrier 2202 further includes a self-sealing material 2206, such as carboxymethyl cellulose (CMC) or cellulose gum, or other sealing material. The air permeable blood barrier 2202 can further include voids 2208 that permit air flow before contact or during partial contact with a fluid such as blood. As shown in FIG. 22B, contact with a fluid causes the self-sealing material 2206 to swell and close off the voids 2208, occluding air flow through the voids 2208 and creating a complete or partial seal.

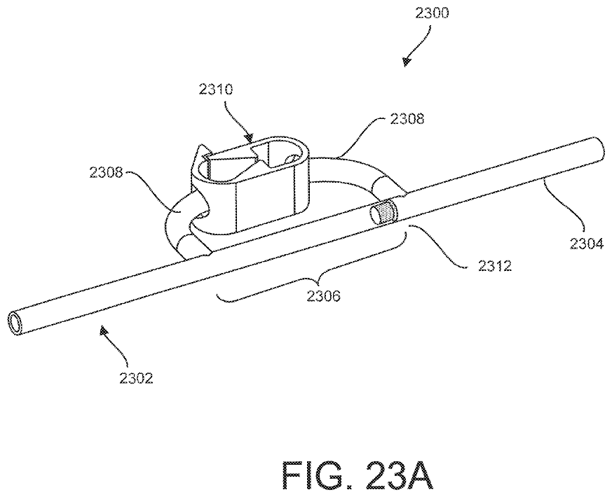

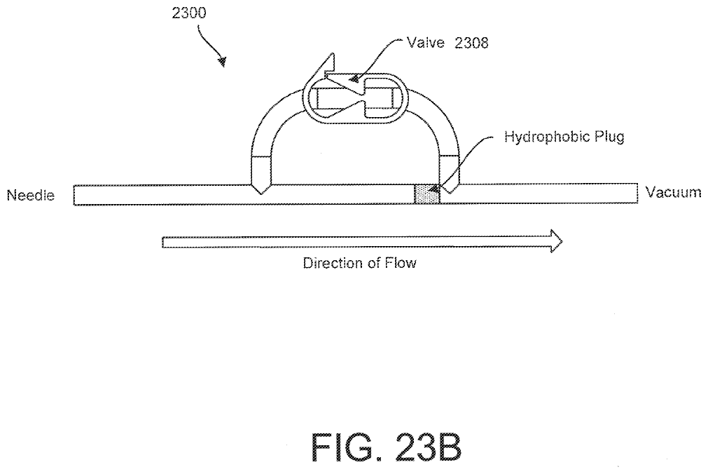

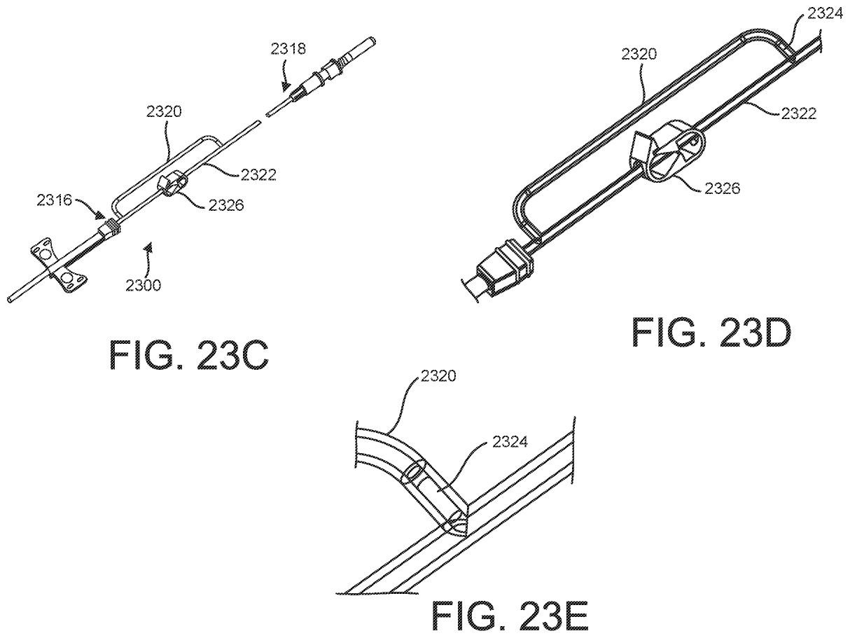

FIGS. 23A and 23B illustrate yet another implementation of a blood sequestration device 2300, having an inlet port 2302 to connect with a patient needle, an outlet port 2304 to connect with a blood sample collection device, a sequestration chamber 2306, and a sampling channel 2308 that bypasses the sequestration chamber 2306 once the sequestration chamber is filled to an initial aliquot of potentially contaminated blood to be sequestered. The sequestration chamber 2306 includes a hydrophobic plug 2312 at a distal end of the sequestration chamber 2306 that is farthest from the inlet port 2302. A vacuum or other drawing force applied from the outlet port 2304, such as from a Vacutainer or the like, draws in blood into the inlet port 2302 and directly into the sequestration chamber 2306, where the initial aliquot of blood will contact the hydrophobic plug 2312 and cause the initial aliquot of blood to back up into the sequestration chamber 2306 and be sequestered there. A small amount of blood may make its way into the sampling channel 2308, which is initially closed off by valve 2308. Upon release of the valve 2308, and under further force of the vacuum or other force, follow-on amounts of blood will flow into inlet port 2302, bypass the sequestration chamber 2306, and flow into and through sampling channel 2308 toward the outlet port 2304 and to the collection device.

The sampling channel 2308 can have any suitable geometry and can be formed of plastic tubing or any other suitable material. Valve 2308 can be a clip or other enclosing device to pinch, shunt, bend or otherwise close off the sampling channel 2308 before the initial aliquot of blood is sequestered in the sequestration chamber 2306. For instance, valve 2308 can also be formed as a flap, door or closable window or barrier within the sampling channel 2308.

FIGS. 23C-23E illustrate an alternative implementation of the blood sequestration device 2300′, in which a sequestration chamber 2320 branches off from a main collection channel 2322 between an inlet port 2316 to connect with a patient needle and an outlet port 2318 to connect with a blood sample collection device, such as a Vacutainer, a syringe, or the like. The sequestration chamber 2320 includes an air-permeable, blood impermeable blood barrier 2324, such as a hydrophobic plug of material, or a filter formed of one or more layers, for example. A valve 2324 closes off and opens the collection channel 2322, and the device 2300′ can be used similarly as described above.

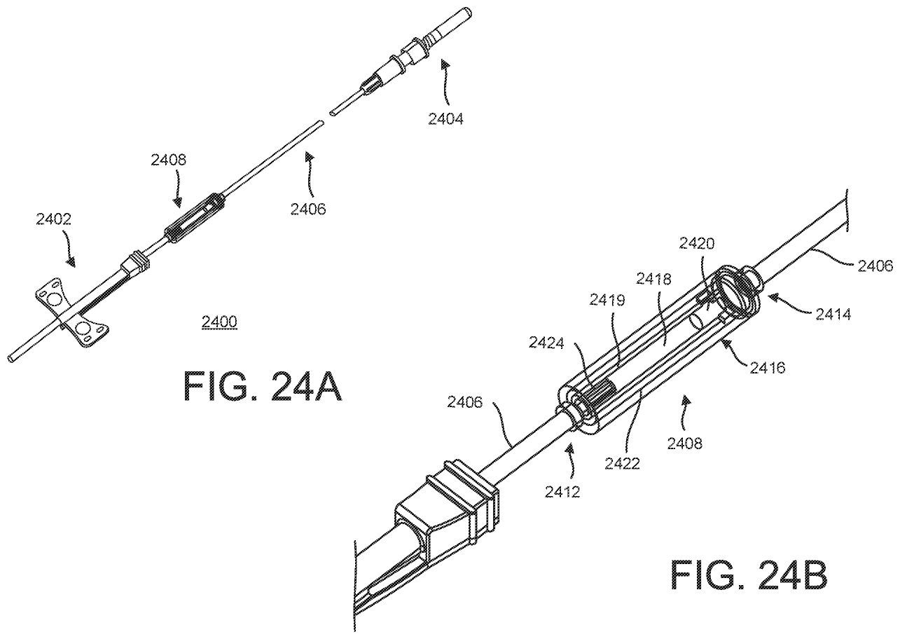

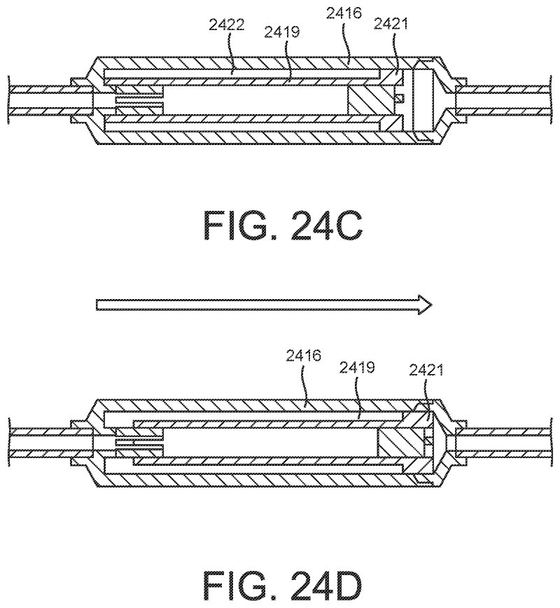

FIG. 24A-24D illustrate a blood sample optimization system 2400 that includes a patient needle 2402 for vascular access to a patient's bloodstream, a blood sample collection device 2404 to facilitate the collecting of one or more blood samples for blood testing or blood cultures, and a conduit 2406 providing a fluid connection between the patient needle 2402 and the blood sample collection device 2404. In some implementations, the blood sample collection device 2404 includes a protective shield that includes a sealed collection needle on which a sealed vacuum-loaded container is placed, which, once pierced by the collection needle, draws in a blood sample under vacuum pressure or force through the conduit 2006 from the patient needle 2402.

The blood sample optimization system 2400 further includes a blood sequestration device 2408, located at any point on the conduit 2406 between the patient needle 2402 and the blood sample collection device 2404. The location of the blood sequestration device 2408 can be based on a length of the conduit between the blood sequestration device 2408 and the patient needle 2402, and the associated volume that length provides.

The blood sequestration device 2408 includes an inlet port 2412 for being connected to the conduit 2406 toward the patient needle 2402, and an outlet port 2414 for being connected to the conduit 2406 toward the blood sample collection device 2404, and a housing 2416. The housing 2416 can be any shape, although it is shown in FIGS. 24A-D as being substantially cylindrical, and includes the inlet port 2412 and outlet port 2414, which can be located anywhere on the housing although shown as being located on opposite ends of the housing 2416.

The blood sequestration device 2408 further includes a blood sequestration chamber 2418 connected with the inlet port 2412. The blood sequestration chamber 2418 is defined by an inner chamber housing 2419 that is movable from a first position to receive and sequester a first aliquot of blood, to a second position to expose one or more apertures 2424 at a proximal end of the inner chamber housing 2419 to allow blood to bypass and/or flow around the inner chamber housing 2419 and through a blood sample channel 2422 defined by the outer surface of the inner chamber housing 2419 and the inner surface of the housing 2416. The blood sequestration chamber 2418 includes an air permeable blood barrier 2420 at a distal end of the blood sequestration chamber 2418.

In operation, the inner chamber housing 2419 is in the first position toward the inlet port 2412, such that the one or more apertures 2424 are closed, and the blood sequestration chamber 2418 is in a direct path from the patient needle. Upon venipuncture of a patient, and drawing of blood by way of a syringe or Vacutainer, or other blood collection device 2404, the initial aliquot of blood flows into the blood sequestration chamber 2418. As the initial aliquot of blood flows into the blood sequestration chamber, it displaces air therein and eventually the blood contacts the blood barrier 2420, forcing the inner chamber housing to the second position. The inner chamber housing 2419 and/or housing 2416 can include a locking mechanism of one or more small tabs, grooves, detents, bumps, ridges, or the like, to maintain the inner chamber housing 2419 in the first position until the blood sequestration chamber 2418 is filled, providing force to overcome the locking mechanism to enable movement of the inner chamber housing 2419 to the second position. Once in the second position, the initial aliquot of blood is sequestered in the blood sequestration chamber 2418 and the one or more apertures 2424 are opened to create a pathway from the inlet port 2412 to the blood sampling channel 2422, bypassing and/or flowing around the blood sequestration chamber 2418.

As described above, the housing 2416 and/or inner chamber housing 2419 can be formed as cylindrical and concentric, but can be any shape, such as squared, rectangular, elliptical, oval, or other cross-sectional shape. The outer surface of the distal end of the inner chamber housing 2419 can have one or more outwardly projecting tangs 2421 with gaps therebetween. The tangs 2421 contact the inner surface of the housing 2416 to help define the blood sampling channel 2422 therebetween, and to help stop the inner chamber housing 2419 in the second position. The gaps between the tangs 2421 enable blood to flow through the blood sampling channel 2422 and to the outlet port 2414. When the inner chamber housing 2419 is in the second position and the blood sequestration chamber 2418 is filled with the first aliquot of blood, further blood samples will automatically flow through the inlet port 2412, through the one or more apertures 2424, through the blood sampling channel 2422, through the gaps between the tangs 2421, and ultimately through the outlet port 2414 to be collected by a blood sampling device 2404.

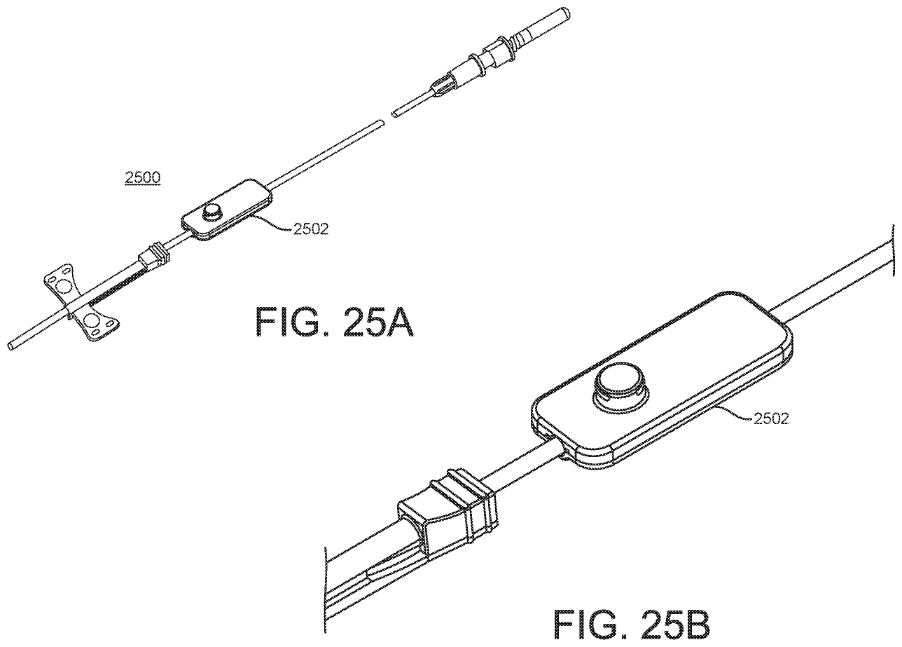

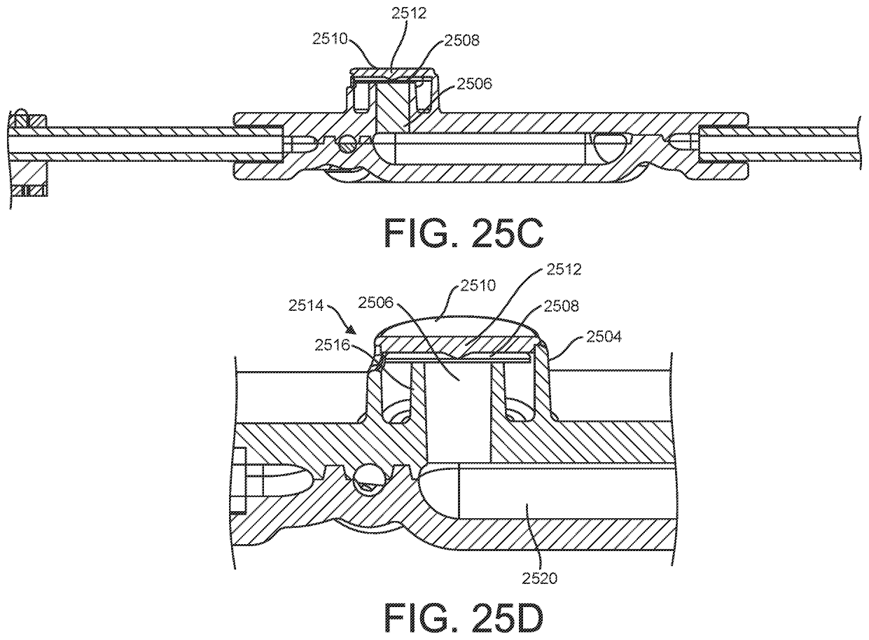

FIGS. 25A-D show a blood optimization system 2500 and blood sequestration device 2502, formed substantially as described in FIGS. 15, 16, 17, 18 and 19, but being formed to inhibit a user or other object from touching or blocking an air venting mechanism from a blood sequestration chamber 2520. Air initially in the blood sequestration chamber 2520 is displaced by an initial aliquot of blood upon venipuncture, where a patient's blood pressure overcomes the ambient air pressure in the blood sequestration chamber 2520. The air venting mechanism includes an air permeable blood barrier 2506, such as a porous material or set of materials that allows air to escape but blocks blood from leaving the blood sequestration chamber 2520.

The air venting mechanism includes an inner wall 2516 that at least partially circumscribes or surrounds the air permeable blood barrier 2506, and an outer wall 2504 spaced apart from the inner wall 2516. The outer wall 2504 can have one or more air vents 2514 formed therein. The outer wall 2504 extends higher upward than the inner wall 2516, such that a lid 2510, such as a cap, plug, cover, etc., can be attached to the outer wall 2504 and be displaced by a small distance from the top of the inner wall 2516. A seal 2508 in the form of a silicone wafer, or other elastomeric material, fits within the outer wall 2504 to cover the air permeable blood barrier 2506 and abut the top of the inner wall 2516. The seal 2508 covers and seals the air permeable blood barrier 2506 and inhibits air from entering the blood sequestration chamber 2520 through the air permeable blood barrier 2506. A fulcrum 2512 on an underside of the lid 2510 allows the seal 2508 to flexibly disconnect from the top of the inner wall 2516 when pushed by air displaced from the blood sequestration chamber 2520, to allow air to vent from the air permeable blood barrier 2506 and through the one or more air vents 2514 in the outer wall 2504.

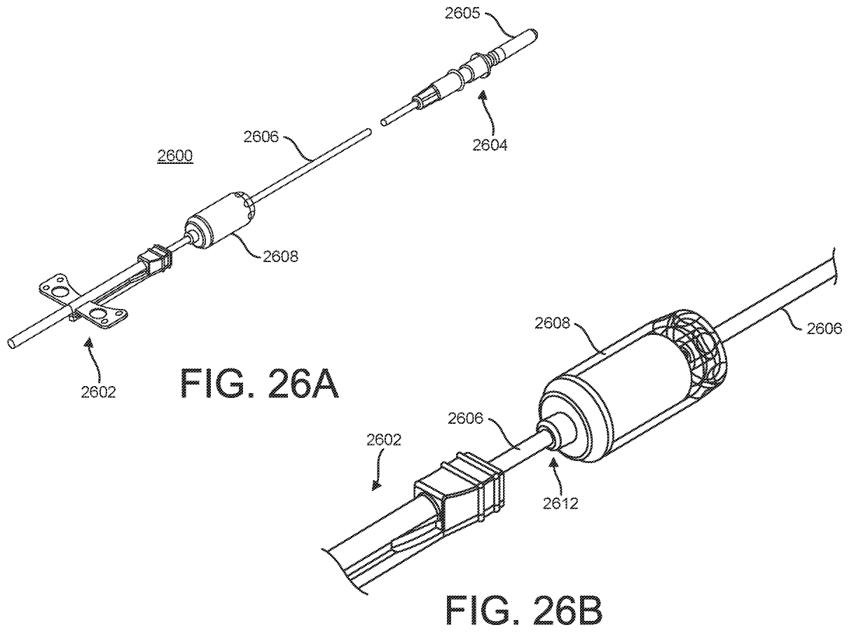

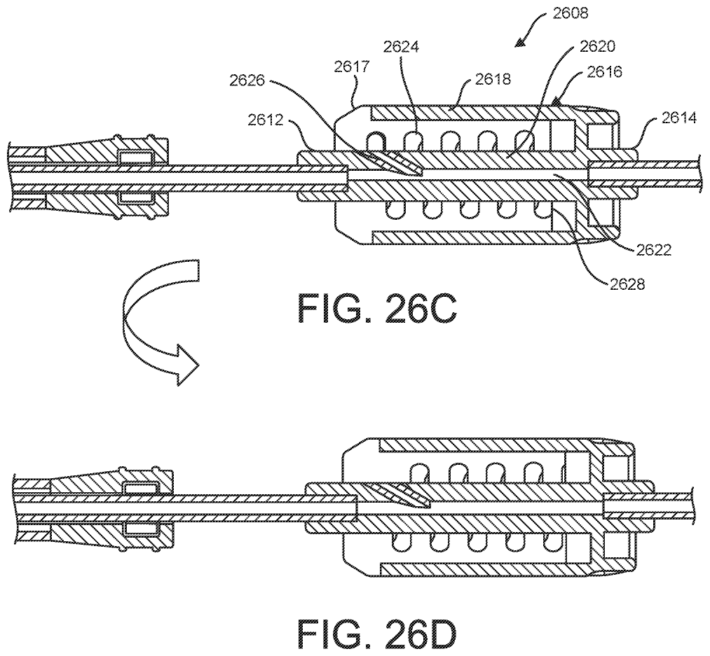

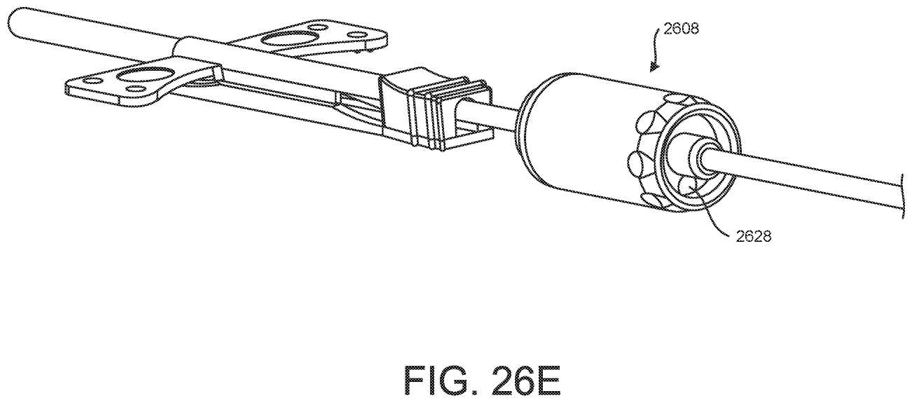

FIG. 26A-E illustrate a blood sample optimization system 2600 that includes a patient needle 2602 for vascular access to a patient's bloodstream, a blood sample collection device 2604 to facilitate the collecting of one or more blood samples for blood testing or blood cultures, and a conduit 2606 providing a fluid connection between the patient needle 2602 and the blood sample collection device 2604. The conduit 2606 can include flexible tubing. In preferred implementations, the blood sample collection device 2604 includes a protective shield 2605 that includes a sealed collection needle on which a sealed vacuum-loaded container is placed, which, once pierced by the collection needle, draws in a blood sample under vacuum pressure or force through the conduit 2006 from the patient needle 2602.

The blood sample optimization system 2600 further includes a blood sequestration device 2608, located at any point on the conduit 2606 between the patient needle 2602 and the blood sample collection device 2604. The location of the blood sequestration device 2608 can be based on a length of the conduit between the blood sequestration device 2608 and the patient needle 2602, and the associated volume that length provides.

The blood sequestration device 2608 includes an inlet port 2612 for being connected to the conduit 2606 toward the patient needle 2602, and an outlet port 2614 for being connected to the conduit 2606 toward the blood sample collection device 2604. The blood sequestration device 2608 includes an outer housing 2616 and an inner housing 2617, both having a cylindrical form, and being connected concentrically. The outer housing 2616 includes an outer wall 2618 and an inner conduit 2620 that defines a blood sampling channel 2622 to convey blood through the conduit 2606 to the blood sampling device 2604. The inner housing 2617 fits snugly between the inner conduit 2620 and the outer wall 2618 of the outer housing, and is rotatable in relation to the outer housing 2616. The fit between the outer housing 2616 and the inner housing 2617 can be a friction fit that maintains the housings in a particular position. The inner housing 2617 defines a blood sequestration chamber 2624, preferably a helical or corkscrew channel around the outer surface of inner conduit 2620 of the outer housing 2616, and which terminates at an air vent 2628 having an air permeable blood barrier, as shown in FIG. 26E.

The blood sequestration chamber 2624 is connected with the blood sampling channel 2622 via diversion junction 2624 formed in the inner conduit 2620, when the blood sequestration device in a first state, illustrated in FIG. 26C. The protective shield 2606 on the collection needle 2604 provides a block for air or blood, enabling a diversion of an initial aliquot of blood into the blood sequestration chamber 2624 as the patient's blood pressure overcomes the ambient air pressure in the blood sequestration channel 2624 to displace air therefrom through air vent 2628.

When the inner housing 2617 is rotated relative to the outer housing 2616, or vice versa, to a second state, as illustrated in FIG. 26D, the blood sequestration chamber 2624 is shut off from diversion junction 2624, enabling a direct path from the patient needle through the conduit 2606 to the collection needle 2604, via blood sampling channel 2622. The outer housing 2616 and/or inner housing 2617 can include ridges or grooves formed within a portion of their surfaces, to facilitate relative rotation from the first state to the second state.

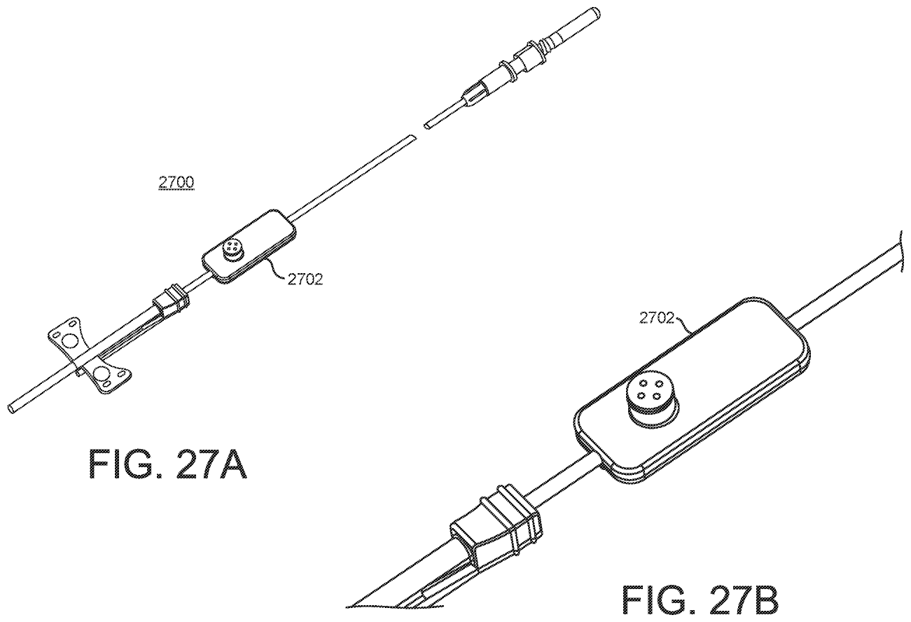

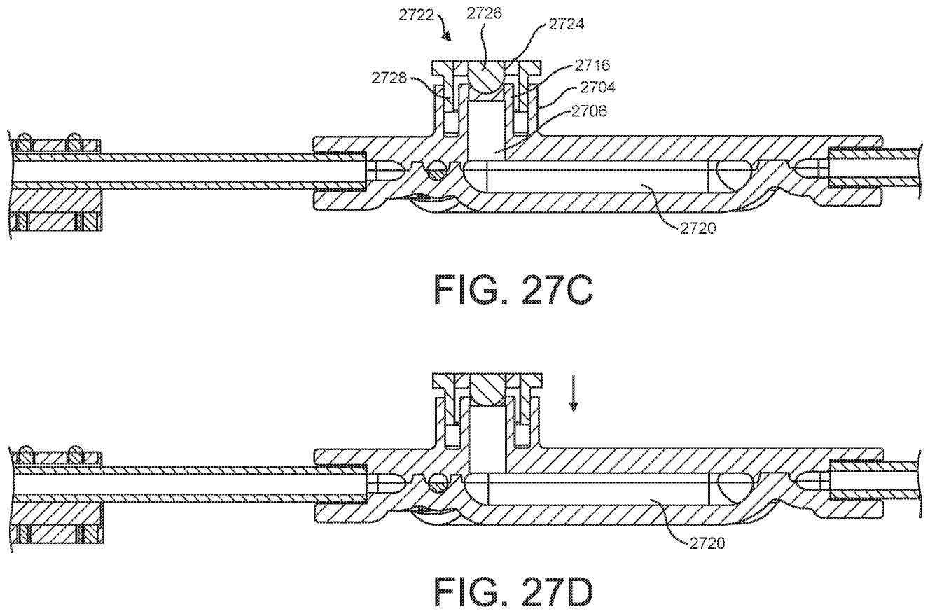

FIGS. 27A-D illustrate a blood optimization system 2700 and blood sequestration device 2702, formed substantially as described with reference to at least FIGS. 15, 16, 17, 18, 19, and 25, but being formed to inhibit a user or other object from touching or blocking an air venting mechanism from a blood sequestration chamber 2720. Air initially in the blood sequestration chamber 2720 is displaced by an initial aliquot of blood upon venipuncture, where a patient's blood pressure overcomes the ambient air pressure in the blood sequestration chamber 2720. The air venting mechanism includes an air permeable blood barrier 2706, such as a porous material or set of materials that allows air to escape but blocks blood from leaving the blood sequestration chamber 2720.

The air venting mechanism includes an inner wall 2716 that at least partially circumscribes or surrounds the air permeable blood barrier 2706, and an outer wall 2704 spaced apart from the inner wall 2716. A cap 2722 is positioned on the air venting mechanism, preferably by having a lower cap wall 2728 that fits between the inner wall 2716 and the outer wall 2704 of the air venting mechanism, and frictionally abutting either the inner wall 2716 or the outer wall 2704 or both. The cap 2722 further includes one or more vent holes 2724 or slits, apertures, openings, or the like, which extend through an upper surface of the cap 2722 around a downwardly extending plug 2726. The plug 2726 is sized and adapted to fit snugly within the space defined by inner wall 2716.

In a first position, as illustrated in FIG. 27C, the cap 2722 is extended from the air venting mechanism to allow air from the blood sequestration chamber 2720 to exit through the air permeable blood barrier 2706 and through the one or more vent holes 2724. Once the air from the blood sequestration chamber 2720 has been displaced, i.e., when the blood sequestration chamber 2720 is filled with the first aliquot of potentially tainted blood from the patient, then the cap 2722 can be pushed down on the air venting mechanism in a second position as shown in FIG. 27D, so that the plug 2726 fits within the inner wall 2716 over the air permeable blood barrier 2706 to seal the air venting mechanism. In either the first position or the second position, the cap 2722 protects the air permeable blood barrier 2706 from outside air or from being touched by a user.

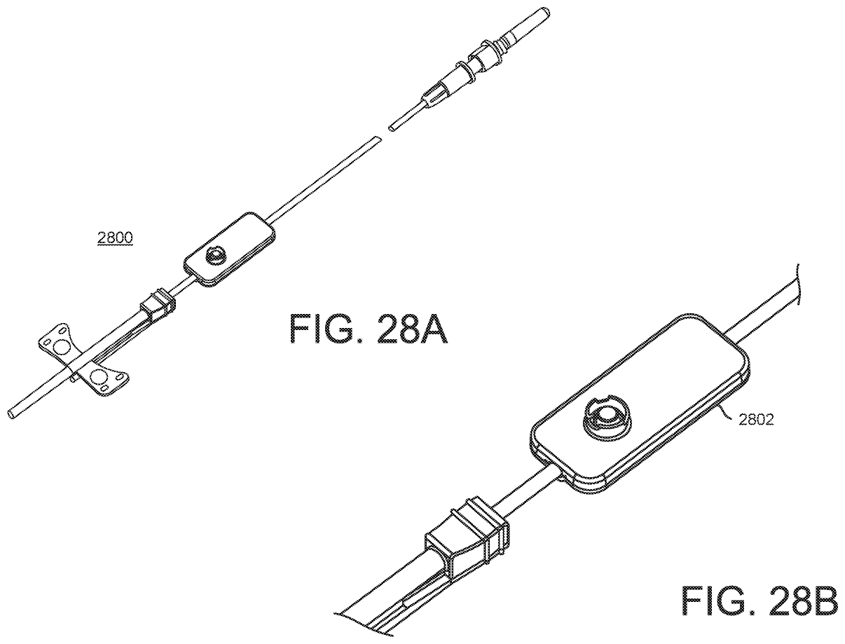

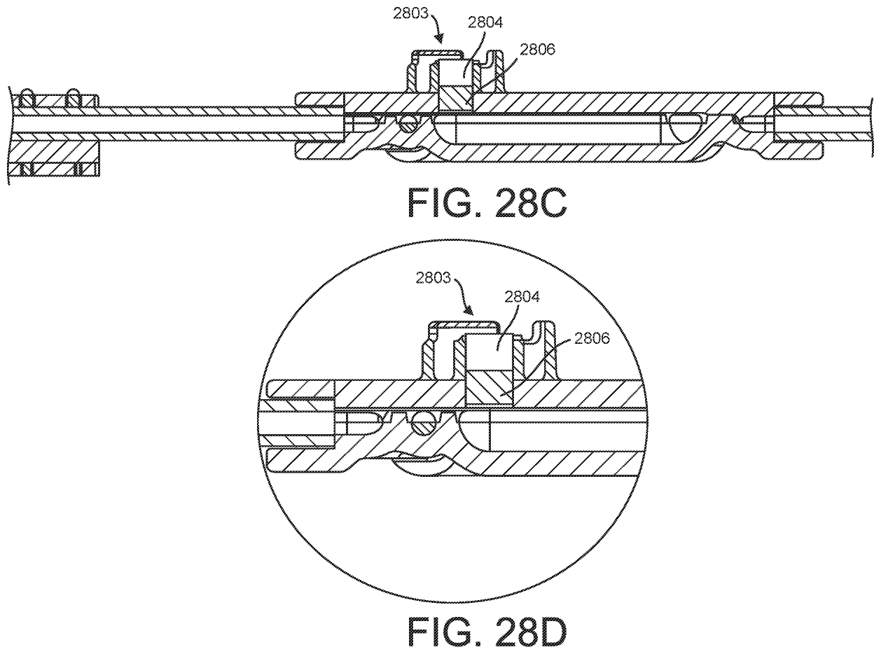

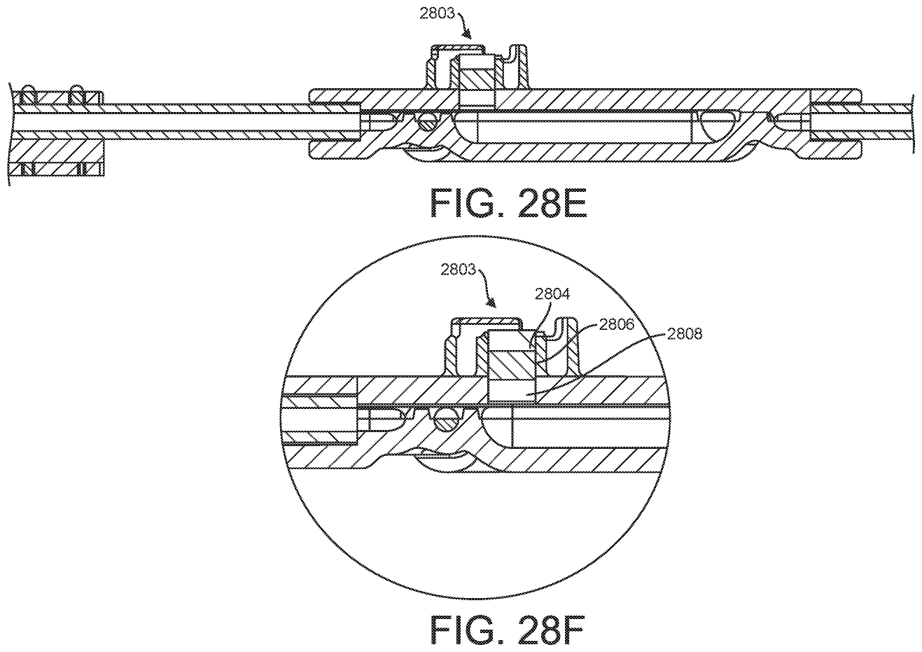

FIGS. 28A-F illustrate a blood optimization system 2800 and blood sequestration device 2802, formed substantially as described with reference to at least FIGS. 15, 16, 17, 18, 19, 25 and 26, but utilizing a multi-layered filter, and in some implementations, a filter with trapped reactive material, for an air permeable blood barrier. As shown in FIGS. 28C and D, an air permeable blood barrier 2803 includes a first layer 2804 of an air permeable but blood impermeable material, and a second layer 2806 that includes a reactive material, such as a hydrophobic material, for repelling blood while still allowing air to pass through both layers. As shown in FIGS. 28E and F, the air permeable blood barrier 2803 can include any number of layers, such as a third layer 2808 formed of the same air permeable but blood impermeable material as first layer 2804, while a second layer 2806 includes trapped or embedded blood reactive material.

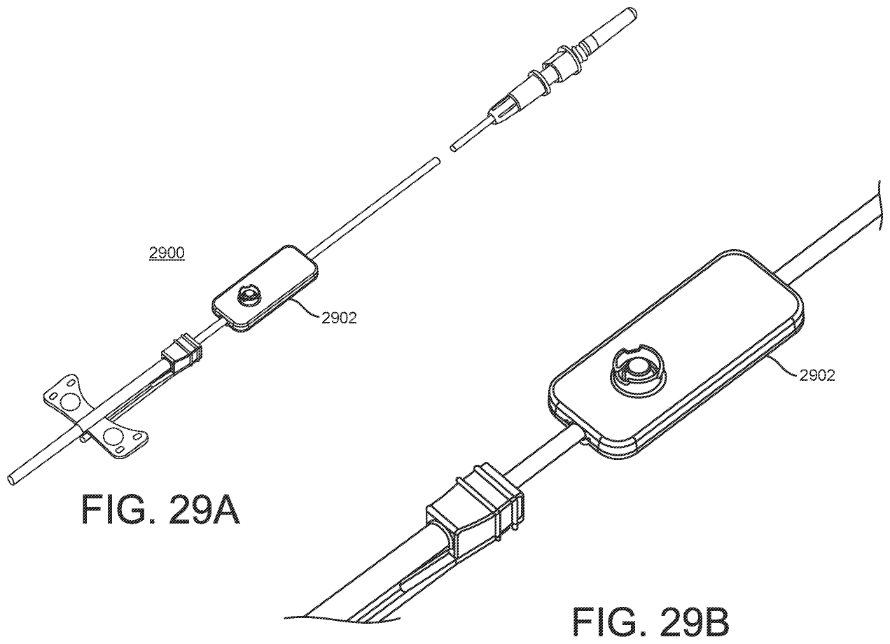

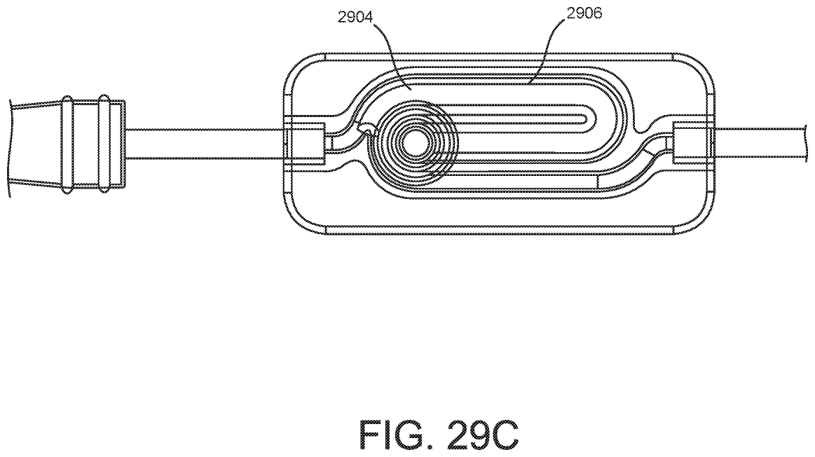

FIGS. 29A-29C illustrate a blood optimization system 2900 and blood sequestration device 2902, formed substantially as described with reference to at least FIGS. 15, 16, 17, 18, 19, 25 and 26, but in which a blood sequestration chamber 2904 is at least partially filled with a blood-absorptive material 2906. The blood-absorptive material 2906 can act as a wicking material to further draw in blood to be sequestered upon venipuncture of the patient, and prior to use of a blood drawing device such as a Vacutainer™ or a syringe, or the like.

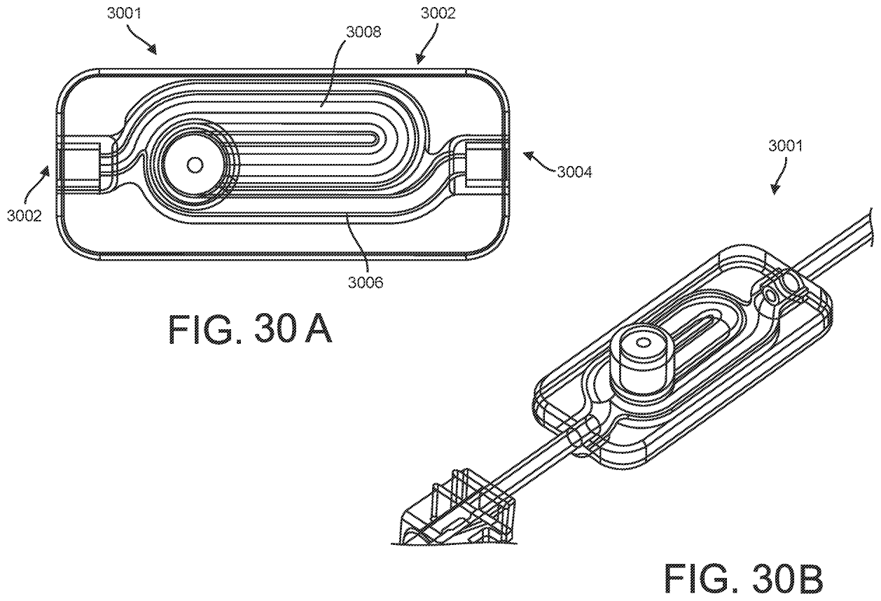

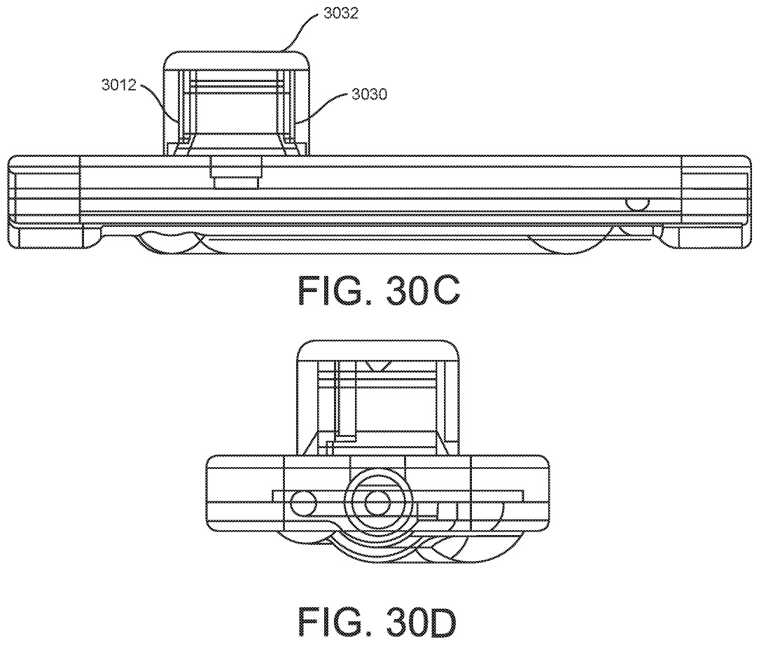

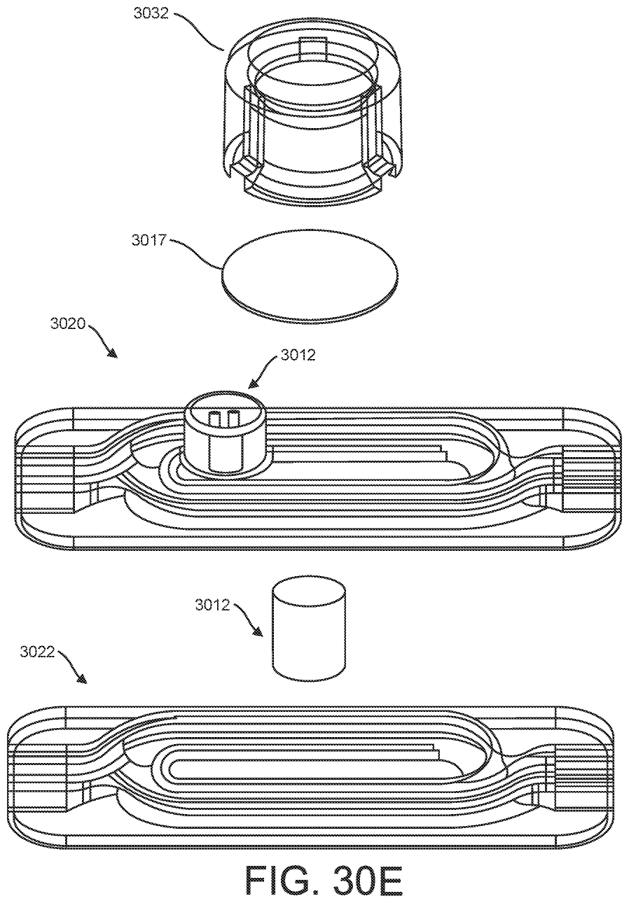

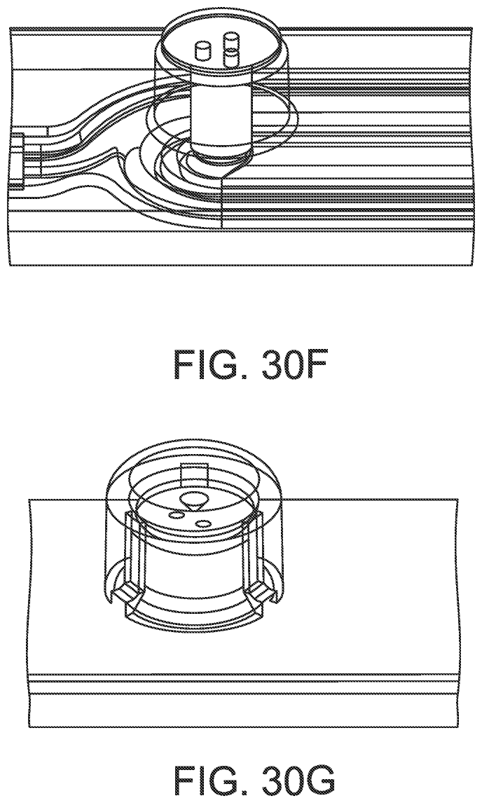

FIGS. 30A-G illustrate a blood optimization system 3000 and blood sequestration device 3002, formed substantially as described with reference to at least FIGS. 15, 16, 17, 18, 19, 25 and 26. The blood sequestration device 3000 includes an inlet port 3002 that can be connected with a patient needle that is inserted into a patient's vascular system for access to and withdrawing of a blood sample. The inlet port 3002 may also be connected with tubing or other conduit that is in turn connected with the patient needle. The inlet port 3002 defines an opening into the blood sequestration device 3000, which opening can be the same cross sectional dimensions as tubing or other conduit connected with the patient needle or the patient needle itself. For instance, the opening can be circular with a diameter of approximately 0.045 inches, but can have a diameter of between 0.01 inches or less to 0.2 inches or more.

The inlet port 3002 can also include a sealing or fluid-tight connector or connection, such as threading or Luer fitting, or the like. In some implementations, tubing or other conduit associated with the patient needle can be integral with the inlet port 3002, such as by co-molding, gluing, laser weld, or thermally bonding the parts together. In this manner, the blood sequestration device 3000 can be fabricated and sold with the patient needle and/or tubing as a single unit, eliminating the need for connecting the patient needle to the blood sequestration device 3000 at the time of blood draw or sampling.

The blood sequestration device 3000 further includes an outlet port 3004, which defines an opening out of the blood sequestration device 3000 and to the blood sample collection device. The outlet port 3004 may also be connected with tubing or other conduit that is in turn connected with the blood sequestration device, and may also include a sealing or fluid-tight connector or connection, such as threading or Luer fitting, or the like. Accordingly, as discussed above, the blood sequestration device 3000 can be fabricated and sold with the patient needle and/or tubing and the blood sample collection device as a single unit, eliminating the need for connecting the patient needle and the blood sample collection device to the blood sequestration device 3000 at the time of blood draw or sampling.

The blood sequestration device 3000 further includes a sampling channel 3006 between the inlet port 3002 and the outlet port 3004, and a sequestration chamber 3008 that is connected to and split off or diverted from the sampling channel 3006 at any point between the inlet port 3002 and the outlet port 3004. The sampling channel 3006 functions as a blood sampling pathway once a first aliquot of blood has been sequestered in the sequestration chamber 3008. The sampling channel 3006 can be any sized, shaped or configured channel, or conduit. In some implementations, the sampling channel 3006 has a substantially similar cross sectional area as the opening of the inlet port 3002. In other implementations, the sampling channel 3006 can gradually widen from the inlet port 3002 to the outlet port 3004. The sequestration chamber 3008 may have a larger cross section to form a big reservoir toward the sequestration channel path so that the blood will want to enter the reservoir first versus entering a smaller diameter on the sampling channel 3006.

In some exemplary implementations, the diversion between the sampling channel 3006 and the sequestration chamber 3008 is by diverter junction 3007. Diverter junction 3007 may be a substantially Y-shaped, T-shaped, or U-shaped. In some preferred exemplary implementations, and as shown in FIG. 17A-17B, the diverter junction 3007 is configured such that the flow out of the inlet port 3002 is preferentially directed toward the sequestration chamber 3008. The sequestration chamber 3008 may also include or form a curve or ramp to direct the initial blood flow toward and into the sequestration chamber 3008.

The sequestration chamber 3008 is preferably maintained at atmospheric pressure, and includes a vent 3010 at or near a distal end of the sequestration chamber 3008. The vent 3010 may include an air permeable blood barrier 3012 as described above.

The blood sequestration device 3000 can include a housing 3001 that can be formed of multiple parts or a single, unitary part. In some implementations, and as illustrated FIG. 30F, the housing 3001 includes a top member 3020 and a bottom member 3022 that are mated together. The blood sequestration device 3000 can also include a gasket or other sealing member (not shown) so that when the top member 3020 is mechanically attached with the bottom member 3022, the interface between the two is sealed by the gasket or sealing member. The bottom member 3022 can include grooves, channels, locks, conduits or other pathways pre-formed therein, such as by an injection molding process or by etching, cutting, drilling, etc., to form the sampling channel 3006, the sequestration chamber 3008, and diverter junction 3007.

The sequestration chamber 3008 may have a larger cross section than the sampling channel 3006 so that the blood will preferentially move into the sequestration chamber first versus entering a smaller diameter on the sampling channel 3006.

In some implementations, the sampling channel 3006 and the sequestration chamber 3008 are formed by grooves, channels, locks or other pathways formed in housing 3001. The housing 3001 can be made of rubber, plastic, metal or any other suitable material. The housing 3001 can be formed of a clear or translucent material, or of an opaque or non-translucent material. In other implementations, the housing 3001 can be mostly opaque or non-translucent, while the housing surface directly adjacent to the sampling channel 3006 and/or the sequestration chamber 3008 may be clear or translucent, giving a practitioner a visual cue or sign that the sequestration chamber 3008 is first filled to the extent necessary or desired, and/or then a visual cue or sign that the sequestered blood remains sequestered while a clean sample of blood is drawn through the sampling channel 3006. Other visual cues or signs of the sequestration can include, without limitation: the air permeable blood barrier 3012 turning a different color upon contact, saturation, or partial saturation with blood; a color-coded tab or indicator at any point along or adjacent to the sequestration chamber; an audible signal; a vibratory signal; or other signal.

The air permeable blood barrier 3012 can be covered with, or surrounded by, a cap 3032. The cap 3032 can be sized and configured to inhibit a user from touching the air permeable blood barrier 3012 with their finger or other external implement, while still allowing air to exit the air permeable blood barrier 3012 as the air is displaced from the sequestration chamber 3008. The cap 3032 can be constructed to inhibit or prevent accidental exposure of the filter to environmental fluids or splashes. This can be accomplished in a variety of mechanical ways including but not limited to the addition of a hydrophobic membrane to the protective cover.

The air venting mechanism includes a wall 3030 that at least partially circumscribes or surrounds the air permeable blood barrier 3012. The wall 3030 can have one or more air vents formed therein. The cap 3032 covers wall 3030 and can be snapped, glued, or otherwise attached in place. A seal 3017 in the form of a silicone wafer, or other elastomeric material, fits within the wall 3030 to cover the air permeable blood barrier 3012 and abut the top of the wall 3030. The seal 3017 covers and seals the air permeable blood barrier 3012 and inhibits air from entering the blood sequestration chamber 3008 through the air permeable blood barrier 3012. A fulcrum 3012 on an underside of the cap 3032 allows the seal 3008 to flexibly disconnect from the top of the inner wall 3016 when pushed by air displaced from the blood sequestration chamber 3008, to allow air to vent from the air permeable blood barrier 3012 and through the one or more air vents in the wall 3030 and/or cap 3032.

In use, the blood sequestration device 3000 includes a sampling channel 3006 and a sequestration chamber 3008. Both pathways are initially air-filled at atmospheric pressure, but the sampling channel 3006 is directed to an outlet port 3004 that will be initially sealed by a Vacutainer or other such sealed blood sampling device, and the sequestration chamber 3008 terminates at a vent 3010 to atmosphere that includes an air permeable blood barrier 3012.

After a venipuncture by a patient needle of a patient (not shown), which could gather a number of pathogens from the patient's skin, a first amount of the patient's blood with those pathogens will pass through inlet port 3002 of blood sequestration device 3000. This initial volume of potentially contaminated blood will preferentially flow into the sequestration chamber 3008 by finding the path of least resistance. The patient's own blood pressure overcomes the atmospheric pressure in the vented sequestration chamber 3008 to displace air therein through the air permeable blood barrier 3012, but is not sufficient to overcome the air pressure that builds up in the sealed sampling channel 3006. In various exemplary embodiments, the sequestration chamber 3008 and sampling channel 3006 can be configured such that the force generated by the patient's blood pressure is sufficient to overcome any effect of gravity, regardless of the blood sequestration device's orientation.

Eventually, the sequestration chamber 3008 fills with blood that displaces air through the air permeable blood barrier 3012. Once the blood contacts the air permeable blood barrier, the blood interacts with the air permeable blood barrier 3012 material to completely or partially seal the vent 3010. A signal or indication may be provided that the practitioner can now utilize the Vacutainer or other blood sampling device.

Upon filling the blood sequestration pathway 3008 but prior to use of the Vacutainer or other blood sample collection device, the patient's blood pressure may drive compression of the air in the sampling channel 3006, possibly resulting in a small amount of blood moving past the diversion point into the sampling channel 3006, queuing up the uncontaminated blood to be drawn through the sampling channel 3006.

In yet another aspect, the blood sequestration chamber and/or blood sampling channel, or other component, of any of the implementations described herein, can provide a visually discernable warning or result in a component adapted for operative fluid communication with the flash chamber of an introducer for an intravenous catheter into a blood vessel of a patient. The device and method provides a visually discernable alert when blood from the patient communicates with a test component reactive to communicated blood plasma, to visually change. The reaction with the blood or the plasma occurs depending on one or a plurality of reagents positioned therein configured to test for blood contents, substances or threshold high or low levels thereof, to visually change in appearance upon a result.

In yet other aspects, the blood sequestration chamber and/or blood sampling channel can be sized and adapted to provide a particular volumetric flow of blood, either during the sequestration process and/or the sampling process.

In still yet other aspects, a non-venting bodily fluid sample optimization device and system, for use in a blood sampling or blood culture collection system, is shown and described. In accordance with implementations described herein, a bodily fluid sample optimization device overcomes problems in prior devices that include permanently-attached, fixed-positioned moving parts, such as valves, state-transitioning switches or diverters, or other mechanisms that move, shift or transition from one operating mode to another operating mode, or from one state to another state.

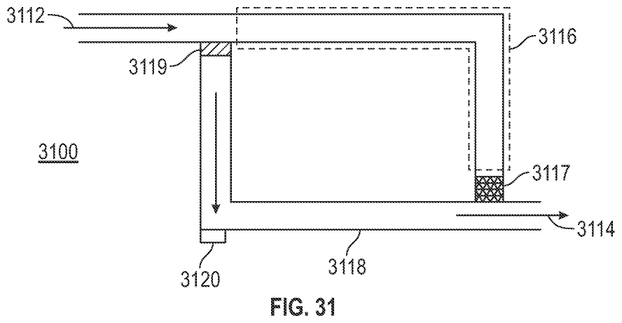

As illustrated in FIG. 31, a fluid sample optimization device 3100 includes an inlet 3112 and an outlet 3114. The inlet 3112 can include an inlet port, connector or interface, for connecting to an external device such as tubing or interface thereof. The inlet 3112 can be connected with a patient or a patient's fluid source, such as via a venipuncture needle, in which fluid is provided at pressure P1 and which can be the patient's blood pressure (which can vary between 0 and 150 mmHg or more).

The outlet 3114 can include an outlet port, connector or interface, for connecting to an external device such as tubing or an interface thereof. For instance, the outlet 3114 can be connected with a fluid collection device, such as an evacuated tube like a Vacutainer® or a syringe, in which fluid is drawn by the fluid collection device from the fluid source by a pressure P2 that is lower than pressure P1, i.e. a negative pressure. The differential pressure between P1 and P2 can provide a motive force for fluid which then allows the fluid sample optimization device 3100 to be closed to atmosphere and atmospheric pressure, i.e. where the fluid sample optimization device 3100 need not include any vent or pathway to outside atmosphere at least when in use.

The fluid sample optimization device 3100 further includes a contaminant containment reservoir 3116 connected with the inlet 3112 and with the outlet 3114, and having an air permeable fluid resistor 3117 between a distal end of the contaminant containment reservoir 3116 and the outlet 3114. As further described herein, the contaminant containment reservoir 3116 can be sized for holding a desired amount of fluid, and may contain an absorbent material that at least partially fills the contaminant containment reservoir 3116. Also as further described herein, the contaminant containment reservoir 3116 can be configured as a tortuous path, a series of chambers of differing cross sections and volumes, and/or contain rifling or baffles extending from an inner surface therein to minimize backflow, i.e. a flow toward the inlet 3112.

The air permeable blood resistor 3117 allows air to pass through and be displaced by a first portion, amount or aliquot of fluid such as blood in the inlet 3112 and sequestration chamber 3116 when a pressure differential is applied between the inlet 3112 and outlet 3114, i.e. a negative pressure at the outlet 3114 is lower than the pressure at the inlet 3112. Once the fluid contacts the air permeable fluid resistor 3117 the flow of fluid into the contaminant containment reservoir 3116 is at least partially stopped, maintaining at least a portion of the fluid in the contaminant containment reservoir 3116.

The fluid sample optimization device 3100 further includes a sample path 3118 also connected with the inlet 3112 and the outlet 3114. The sample path 3118 includes a displaceable plug or stopper 3119 provided in a seat proximate the inlet 3112 in a junction between the inlet and the sample path 3118. The seat can be a portion of the junction, and the displaceable plug 3119 can be friction-fit into the seat. Alternatively, the seat can include a ridge or flange, and the plug can abut such ridge or flange until it is displaced, deflected or compressed by a pressure differential. At the same time the pressure P2 is drawing the first portion or amount of fluid into the contaminant containment reservoir 3116, the displaceable plug 3119 is configured to resist, inhibit, limit or prohibit a flow of the fluid into the sample path 18 until the first portion or amount of fluid has entered into the contaminant containment reservoir 3116, and/or blocked the air permeable fluid resistor 3117.

As described further herein, the displaceable plug 3119 is configured such that after the first portion or amount of fluid has entered into the contaminant containment reservoir 3116 and/or blocked the air permeable fluid resistor 17, the pressure differential increases across the displaceable plug 3119. The higher pressure on the inlet side of the displaceable plug 3119 will cause the displaceable plug 3119 to deflect, at least in some portion of an outer surface, and to dislodge or become loose, and allowing it to get displaced or moved out of its seat and to plug retainer 20. The plug retainer 3120 can be a cavity or chamber that is sized to receive the plug 3119 after it has been displaced, or an extending member that extends from an inner wall of the sample path 3118. The plug retainer 3120 is sized and configured to allow fluid flow without restriction beyond a uniform cross-sectional area of the sample path 3118. Once the displaceable plug 3119 is removed from its seat, a second and/or subsequent portions or amounts of fluid are allowed to flow from the inlet 3112 through the sample path 3118 to the outlet 3114, still under force of the pressure differential between P2 and P1.

A displaceable plug described herein can be formed of any compressible or elastomeric material, such as silicone, EPDM (ethylene propylene diene monomer), or PVC (polyvinyl chloride). The plug can also be made from a more rigid polymer, such as polycarbonate, ABS, acetal, etc. with thin enough walls to form a seal and be deflected from its seat. In addition, the surfaces of the plug that seal against the seat can be lubricated (or the material itself can be impregnated with a lubricious material) to reduce the friction required to displace the plug from its seat when exposed to the pressure differential. Any suitable rubber, synthetic rubber, thermoplastic, or other elastomers can be used.

In some implementations, the fluid sample optimization device 3110 can include an acceleration portion between the inlet 3112 and the contaminant containment reservoir 3116 over or near the displaceable plug 3119, to increase the velocity of the fluid, thereby reducing the pressure of the fluid moving through it. This can further help in preferentially directing the first portion or amount of fluid from the inlet to the contaminant containment reservoir by reducing the pressure differential across the displaceable plug prior to complete filling of the contaminant containment reservoir.

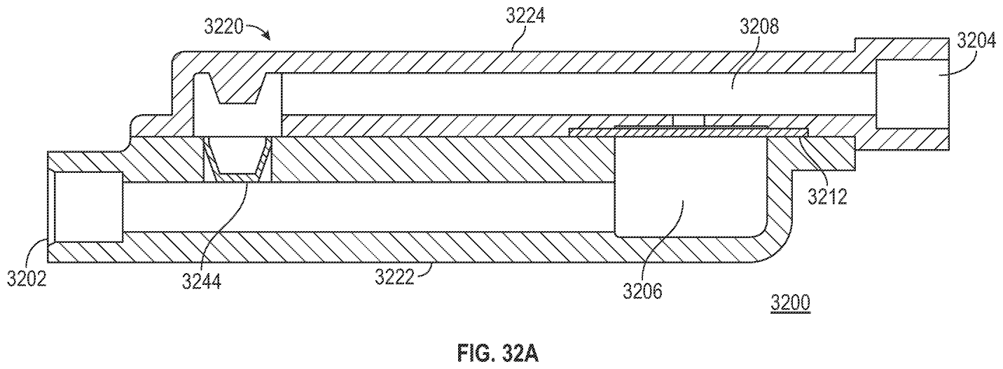

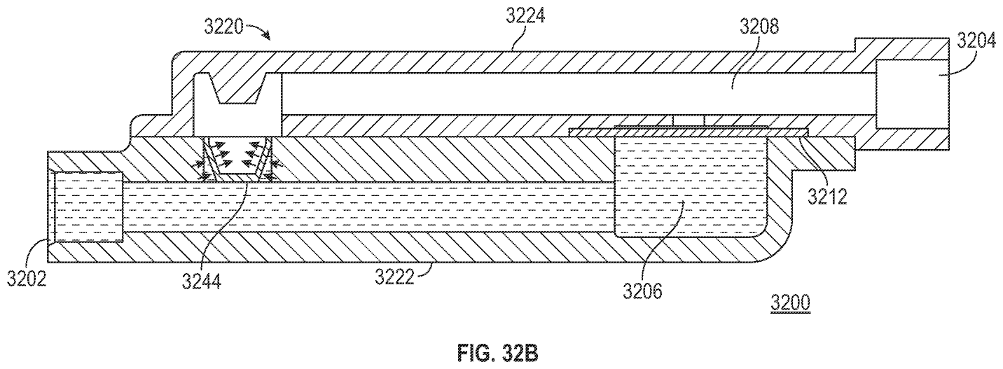

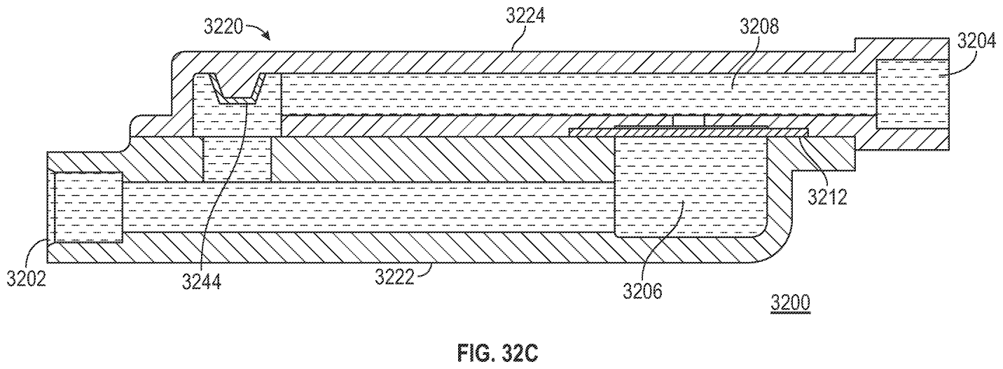

FIGS. 32A-32C illustrate another implementation of a fluid sample optimization device 3200 having just three basic components: 1) a housing 3220, which houses, forms, or defines an inlet 3202, an outlet 3204, a contaminant containment reservoir 3206, and a sampling channel 3208; 2) an air-permeable fluid barrier 3212, positioned in or at a first conduit (hereinafter “first conduit”) between the contaminant containment reservoir 3206 and the sampling channel 3208 proximate the outlet 3204; and 3) a displaceable plug 3214, positioned in or at a second conduit (hereinafter “second conduit”) between the contaminant containment reservoir 3206 and the sampling channel 3208 proximate the inlet 3202.

The inlet 3202 can include an inlet port for connecting to a fluid source, such as a patient needle and tubing. The inlet port can itself include a port connector, such as a Luer locking member, threading, truncated conical opening for a friction fit, or the like. Similarly, the outlet 3204 can include an outlet port for connecting to a fluid collector, such as a Vacutainer®, a syringe, a pump, and associated tubing. The fluid collector provides at the outlet 3204 a vacuum or negative pressure relative to the inlet 3202. The inlet port can itself include a port connector, such as a Luer locking member, threading, truncated conical opening for a friction fit, or the like. Alternatively, the inlet 3202 and/or outlet 3204 can be permanently connected with tubing, such as by glue, heat weld, laser weld, or the like.

The contaminant containment reservoir 3206 is fluidically connected with the inlet 3202, and can include a main reservoir or main basin, and any conduit, channel, pathway between the main reservoir or basin and the inlet 3202. In some instances, the contaminant containment reservoir 3206 is formed of a single elongated chamber having an opening connected with the inlet 3202. The contaminant containment reservoir 3206 is fluidically isolated from the outlet 3204 or the sampling channel 3208 proximate the outlet by the air permeable fluid barrier 3212 at the first conduit between the contaminant containment reservoir 3206 and the outlet 3204 or sampling channel 3208 proximate the outlet 3204, and as explained further below, the air permeable fluid barrier will seal upon contact with a first portion of fluid that enters into the contaminant containment reservoir 3206 to displace air therein through the air permeable fluid barrier 3212.

The sampling channel 3208 is fluidically connected with the outlet 3204, and is at least initially sealed from, or not fluidically connected, with the inlet 3204, as the displaceable plug blocks, inhibits, restricts or seals the second conduit between the sampling channel 3208 and the inlet 3202 or the contaminant containment reservoir 3206 proximate the inlet 3202. Preferably, the sampling channel 3208 is formed of or defined as a tube, channel or pathway having any sized- or shaped-cross section or geometry. The sampling channel 3208 can include a protrusion or tang above the displaceable plug 3214, for receiving an holding the displaceable plug 3214 once it is displaced from the second conduit by a pressure differential between the outlet 3204 and the inlet 3202 when the contaminant containment reservoir 3206 receives and contains the first amount of fluid, as will be described in further detail below. Further, the sampling channel 3208 can include one or more blocks, recesses, side channels, cavities, or the like, for receiving the plug 3214.

In some implementations, the housing 3220 can include, or be formed of, a lower housing portion 3222 mated with an upper housing portion 3224, in accordance with an orientation of the device 3200 as shown. The lower housing portion 3222 can include, form, or define the contaminant containment reservoir 3206, the inlet 3202, and a first portion of the first and second conduits. The upper housing portion 3224 can include, form, or define the sampling channel 3208, the outlet 3204, and a second portion of the first and second conduits. The lower housing portion 3222 and upper housing portion 3224 can be mated together and the fluid paths sealed by glue, thermal welding (ultrasonic, laser, friction, etc.), screws, bolts or any other connecting mechanism or process.

As shown in FIG. 2A, when a negative pressure differential is applied between the outlet 3204 and the inlet 3202, a first amount of fluid, which is likely to have contaminants, is “pulled” into the inlet 3202 by the negative pressure and into or toward the contaminant containment reservoir 3206, since the sampling channel 3208 is initially blocked or restricted by displaceable plug 3214. And, because of the presence of the displaceable plug 3214 in the second conduit to the sampling channel 3208, the first amount of fluid bypasses the displaceable plug 3214 and the sampling channel 3208. The negative pressure differential will continue to pull fluid into the contaminant containment reservoir 3206 until all the air therein is displaced by fluid, and the fluid contacts the air permeable fluid barrier 3212, effectively sealing it off from the negative pressure.

Once the contaminant containment reservoir 3206 is filled with the first portion of fluid and the air permeable fluid barrier 3212 is sealed, the full pressure differential between the inlet and outlet is applied across the displaceable plug 3214 (see FIG. 2B), applying a force to the plug 3214 to deform, collapse inward, loosen and then move it from the second conduit to the sampling channel 3208, as shown in FIG. 2C. Once displaced from the second conduit to the sampling channel 3208, and into the proximal end of the sampling channel 3208, displacement of the displaceable plug 3214 can be maintained by a protrusion or tang on an inside surface of the sampling channel 3208 above the second conduit, as shown in FIG. 2C. Displacement of the displaceable plug 3214 then allows subsequent amounts of fluid to bypass the first amount of fluid, enter into and through the sampling channel 3208, and pulled out the outlet 3204. A flow or drawing of the subsequent amounts of fluid from the inlet 3202 into and through the sampling channel 3208 work to keep the plug displaced away from the second conduit. For instance, the displaceable plug 3214 can have a bottom surface that is planar and circular, or slightly curved, so as to facilitate displacement from the second conduit. The curvature can be concave or convex. In some implementations, the bottom surface of the displaceable plug 3214 can be coated with a hydrophobic layer, to facilitate flow of the first portion of fluid past the plug 3214, as well as facilitate flow past the plug 3214 and through the sampling channel 3208 when the plug 3214 is displaced.

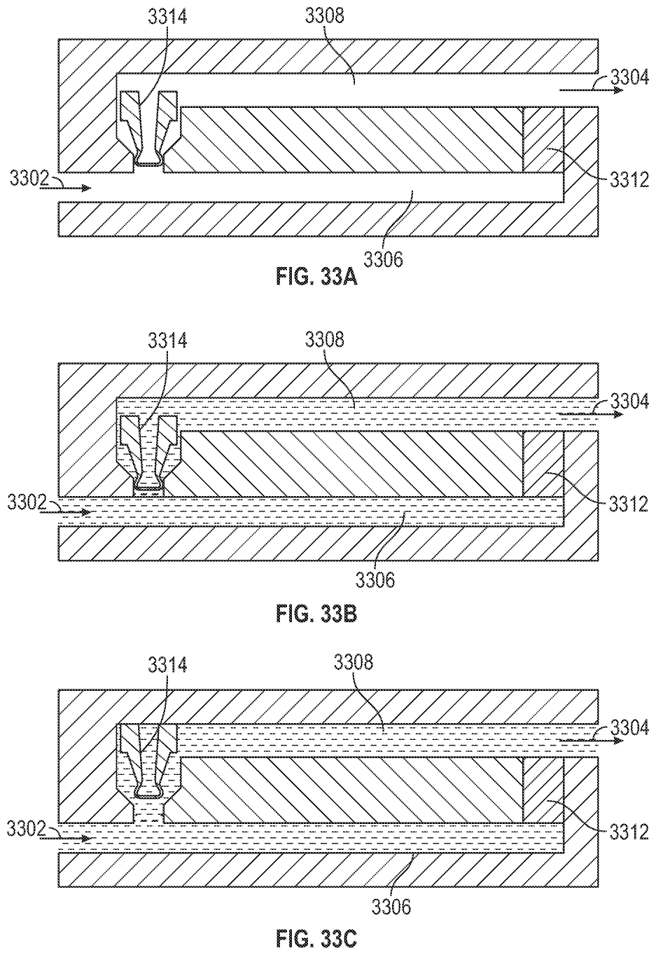

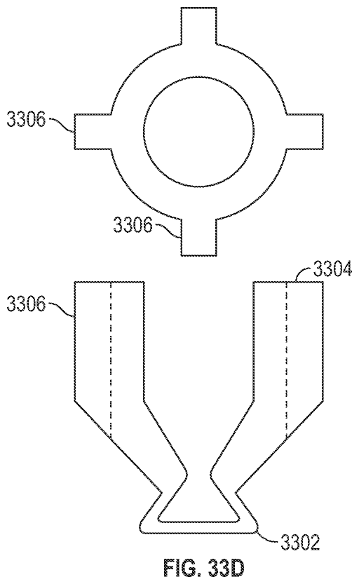

FIGS. 33A-33D illustrate an alternative implementation of a fluid sample optimization device 3300, having an inlet 3302, an outlet 3304. The fluid sample optimization device 3300 further includes a contaminant containment reservoir 3306 fluidically coupled with the inlet 3302 and connected with the outlet 3304 via a first conduit having an air permeable fluid barrier 3312. The fluid sample optimization device 3300 further includes a sampling channel 3308 fluidically coupled with the outlet and connected with the inlet via a second conduit having a displaceable plug 3314 that initially seals the second conduit. The outlet 3304 is fluidically connected with a fluid sampling device that provides a vacuum or negative pressure at the outlet 3304. The inlet is fluidically connected with a fluid source, such as a patient needle configured for venipuncture of a patient.

Upon activation of the vacuum or negative pressure at the outlet 3304, a negative pressure differential is formed between the outlet 3304 and the inlet 3302. As shown in FIG. 3B, the negative pressure from the outlet 3304 draws in fluid into the inlet 3302 and into the contaminant containment reservoir 3306, displacing air through the air permeable membrane 3312 and bypassing the second conduit between the inlet 3302, as the second conduit is blocked by displaceable plug 3314.

Once the initial amount of fluid flows into, and is contained in, the contaminant containment reservoir 3306, the still-present vacuum or negative pressure at the outlet 3304 by a fluid sampling device causes the plug 3314 to be squeezed or otherwise collapsed, which pulls the plug 3314 from the second conduit to open it, as shown in FIG. 3C. This allows subsequent amounts of fluid to be pulled into the inlet 3302, through the second conduit and into the sampling channel 3308, toward and out the outlet 3304.

FIG. 3D illustrates a plug 3314 having a post 3332 that has a cross-sectional area that is smaller than a cross-sectional area of the second conduit. The plug 3334 further includes a hollow or tubular top portion 3334, which is collapsible upon application of a negative pressure on a side of the plug opposite the post 3332. The plug 3334 is configured to collapse upon a minimal threshold of pressure. The collapsing under pressure can be configured by a length of the top portion 3334, a thickness of walls of the top portion 3334, an elasticity of the material that forms the plug 3314, or any combination thereof. The plug 3314 can further include a set of vertical ribs 3336 or protrusion, or the like, for creating a space or conduit therebetween to ensure fluid flow therethrough upon displacement of the plug 3314.

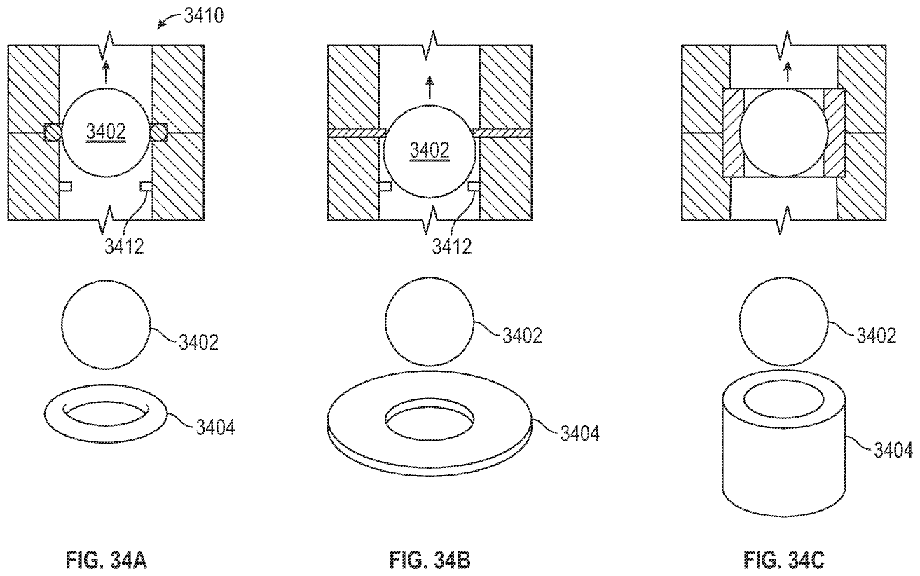

FIGS. 34A-34C illustrate various alternative implementations of a displaceable plug 3402 or stopper, shown in the form of a ball or rounded object (i.e. oblong, or egg-shaped), but which can be any shape, such as cylindrical, bullet-shaped, disk-shaped, a curved cap or planar plug, or any other shape. The plug 3402 is held in place in a junction 3410 of the device by a seat 3404 or seating member until displaced by a pressure differential. The seat can be elastomeric, semi-rigid or rigid. For example, the seat 3404 can be an o-ring for a plug with a circular or semi-circular cross-section (FIG. 34A), a thin sheet with a hole or aperture (FIG. 34B), or a short segment of tubing that holds the plug 3402 in place until displaced (FIG. 34C). The seat 3404 can be held stationary between upper and lower housing members of the device.

In some cases, particularly such as shown in FIGS. 34A and 34B, a feature such as a shelf 3412 or lip in the housing or the sample path or sampling channel can cooperate with the seating member to keep the plug in place, i.e. not allow reverse displacement toward the inlet or contaminant containment reservoir. The dimensions and geometry of the plug, seating member, and/or path in which the seating member and plug reside, can be designed such that the plug will not be pulled through too early—i.e. while the contaminant containment reservoir is filling—but when the contaminant containment reservoir is full and the pressure differential increases across the stopper, the plug will be pulled upward and allow flow past the seating member through the path.

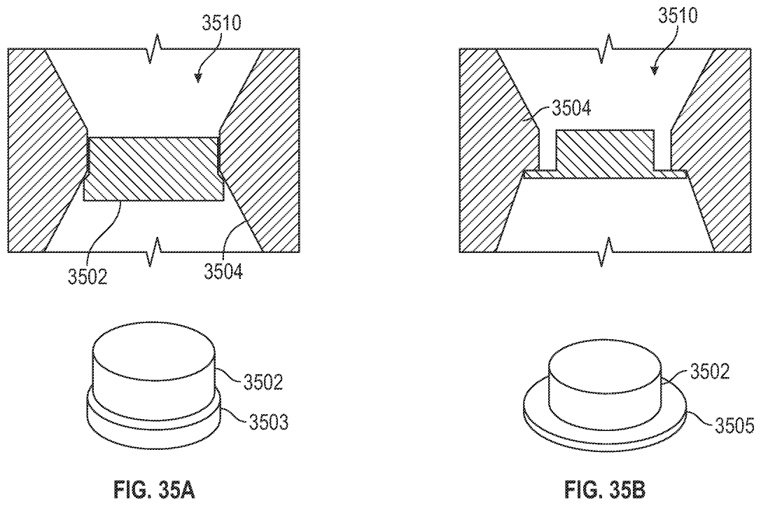

FIGS. 35A and 35B illustrate various alternative implementations of a displaceable plug 3502 or stopper, shown in the form of a disk with a shoulder 3503, 3505 that holds it in place within a junction 3510 or path between the inlet and the sample path or sampling channel, until the pressure differential overcomes the force of the shoulder 3503, 3505 in the junction 3510 to allow the plug 3502 to be displaced and exit its seat in the junction 3510. The path between the inlet and the sample path or sampling channel can include a protrusion 3504, such as a small ring or one or more small tangs or flanges, that mate with the shoulder of the plug until such mating is overcome by pressure.

FIG. 35A illustrates the plug as a one-piece elastomer disk with an enlarged diameter shoulder 3503 that keeps the plug from moving in the path until the pressure differential is applied. FIG. 35B shows the plug as a disk with a thin flexible sheet 3505 attached that would deform when pushed upward. Accordingly, the plug can be formed of one unitary piece of material, or several pieces of material each having different durometers, elasticities or flexibility. For instance, the disk of FIG. 35B can be formed of a rigid material, which can be hollow or solid, and the larger-diameter flexible sheet can be formed of a highly flexible material that is tuned to flex upon exertion of a certain range of pressure on it or the disk.

While FIGS. 35A and 35B illustrate a rounded disk shape, it should be understood that the plug can have any cross-sectional geometry or shape. For example, in some implementations, the flexible sheet or extended ridge can be rounded, while the upper disk or plug member can have one or more angled surfaces, such as a pyramid, square, cone, or the like, that is configured to fit into a corresponding receptacle in the sample path of a similar shape, such as with a friction fit or the like.

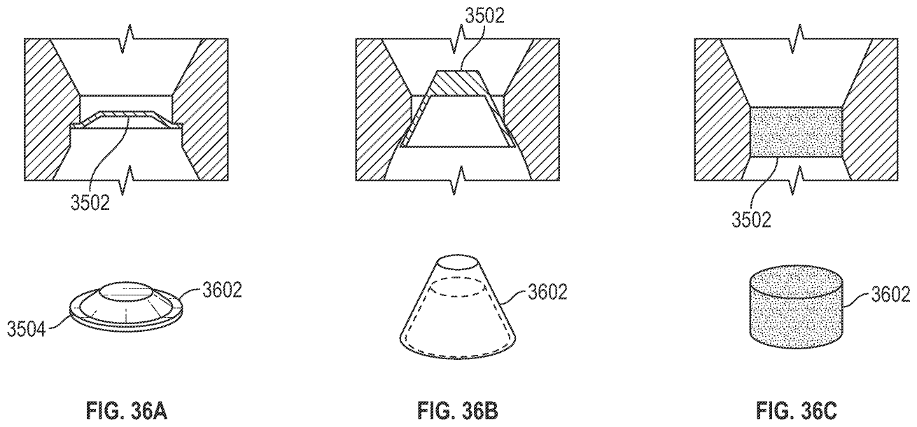

FIGS. 36A-36C illustrate further various alternative implementations of a displaceable plug or stopper, consistent with the devices described herein. FIG. 36A shows a plug in the shape of a thin elastomeric disk, or membrane, with a circumferential o-ring member, such as a gasket, where the circumferential o-ring member has a thickness or cross-section that is larger than a thickness of the disk, and abuts or is set within a seat. In some implementations, the disk can be in the shape of an umbrella. When subjected to a differential pressure (i.e. a relatively higher or positive pressure on the underside of the plug than on the top side of the plug), the membrane will deform and the entire plug will be displaced from its seat. The membrane can be curved, such as curved upward. In some implementations, the plug can include only one or more peripheral abutments or sections.

FIG. 36B shows a plug 3602 being formed as a hollowed-out elastomeric stopper which deforms easily under a threshold amount of pressure, to release the plug 3602 for displacement from its seat. FIG. 36C shows a plug 3602 formed as a soft, compressible material such as a closed-cell foam, and which is press-fit or friction-fit into the junction or path. The plug 3602 can also be formed from an open-cell foam, but preferably covered by a fluid barrier.

One challenge of a device as described herein is providing a location or member to which the plug or stopper can move or couple with so that it does not block the flow through the sample path or move downstream into a collection device, such as a Vacutainer® bottle. In some implementations, a screen or grate can be used or positioned in the sample path downstream from the junction or path seat, and which can catch the stopper after it is displaced from the seat. Alternatively, the shape of the sample path can be configured so as to have a uniform cross-sectional area along the sample path, but which changes shape so as to not allow the plug or stopper to traverse the length of the sample path.

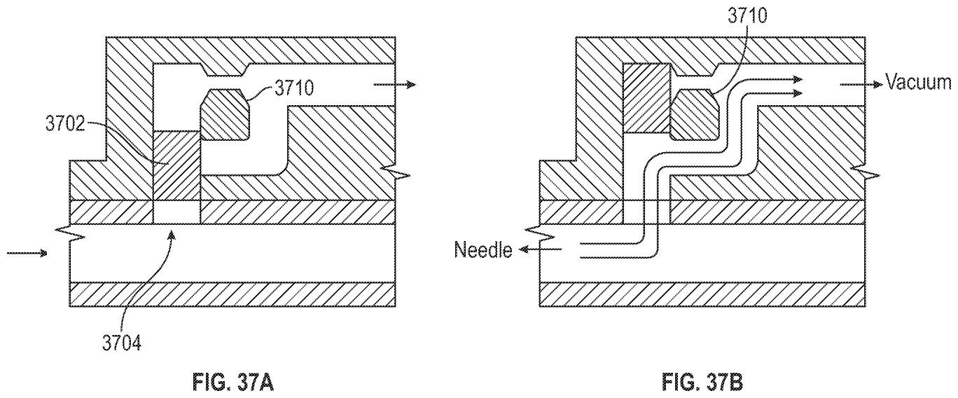

FIGS. 37A and 37B show a variation of a junction into the sample path in which a stopper 3702 or plug, which is not permanently attached to any wall or other structure of the device, moves to a position that allows flow through an alternate path, created by a divider 3710 within the sample path or sampling channel. In some implementations, the housing of the device can be formed to allow a free movement of the stopper 3702 or plug from a seat within a junction between the inlet and the sample path, and a receptacle such as a recess, cavity, pin, or other protrusion, formed on an inner surface of the sample path. Preferably, once the stopper or plug is displaced, the resultant path through the sample path is configured to allow a free flow of fluid, i.e. unimpeded or unrestricted, from the inlet through the sample path.

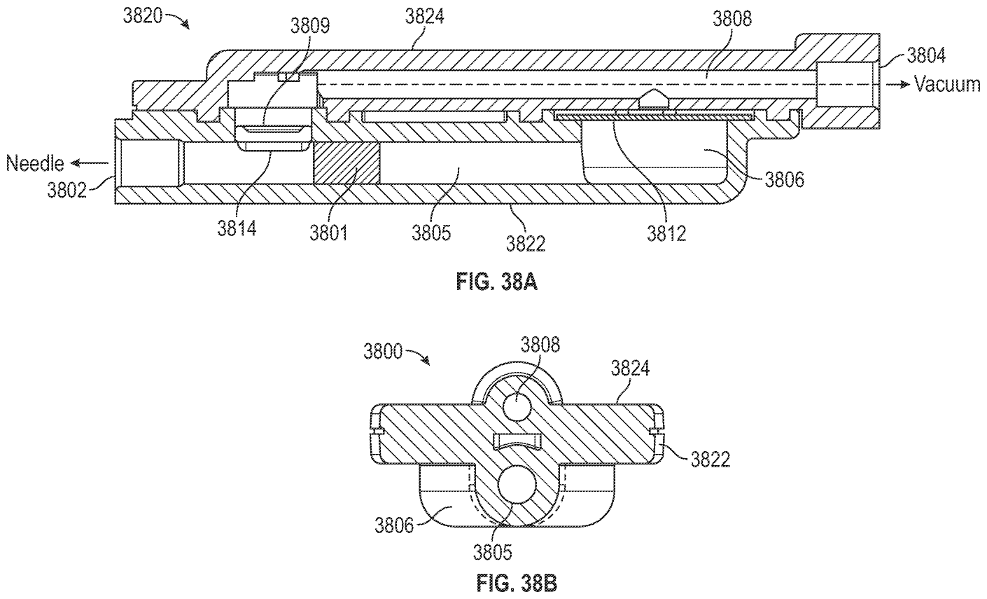

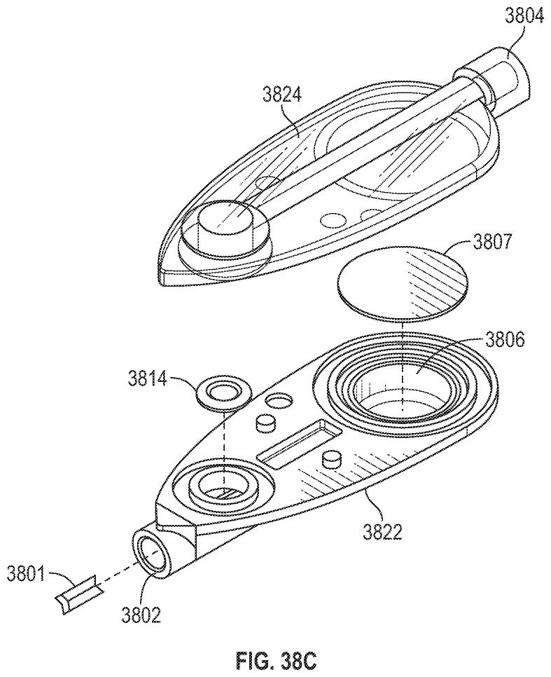

FIG. 38A is a side cross-sectional view, FIG. 38B is front-to-back cross-sectional view, and FIG. 38C is an exploded view of another implementation of a fluid sample optimization device 3800 having: 1) a housing 3820, which houses, forms, or defines an inlet 3802, an outlet 3804, a contaminant containment reservoir 3806, and a sampling channel 3808; 2) an air-permeable fluid barrier 3812, positioned in or at a first conduit between the contaminant containment reservoir 3806 and the sampling channel 3808 proximate the outlet 3804; and 3) a displaceable plug 3814, positioned in or at a second conduit between the contaminant containment reservoir 3806 and the sampling channel 3808 proximate the inlet 3802.

The inlet 3802 can include an inlet port for connecting to a fluid source, such as a patient needle and tubing. The inlet port can itself include a port connector, such as a Luer locking member, threading, truncated conical opening for a friction fit, or the like. Similarly, the outlet 3804 can include an outlet port for connecting to a fluid collector, such as a Vacutainer®, a syringe, a pump, and associated tubing. The fluid collector provides at the outlet 3804 a vacuum or negative pressure relative to the inlet 3802. The inlet port can itself include a port connector, such as a Luer locking member, threading, truncated conical opening for a friction fit, or the like. Alternatively, the inlet 3802 and/or outlet 3804 can be permanently connected with tubing, such as by glue, heat weld, laser weld, or the like.

The contaminant containment reservoir 3806 is fluidically connected with the inlet 3802, and can include a main reservoir and associated conduit, channel, or pathway between the main reservoir and the inlet 3802. In some instances, the contaminant containment reservoir 3806 is formed of a single elongated chamber having an opening connected with the inlet 3802. The contaminant containment reservoir 3806 is fluidically isolated from the outlet 3804 or the sampling channel 3808 proximate the outlet 3804 by the air permeable fluid barrier 3812 at the first conduit between the contaminant containment reservoir 3806 and the outlet 3804 or sampling channel 3808 proximate the outlet 3804, and as explained further below, the air permeable fluid barrier will seal upon contact with a first portion of fluid that enters into the contaminant containment reservoir 3806 to displace air therein through the air permeable fluid barrier 3812.

The sampling channel 3808 is fluidically connected with the outlet 3804, and is at least initially sealed from, or not fluidically connected, with the inlet 3804, as the displaceable plug blocks, inhibits, restricts or seals the second conduit between the sampling channel 3808 and the inlet 3802 or the contaminant containment reservoir 3806 proximate the inlet 3802. Preferably, the sampling channel 3808 is formed of or defined as a tube, channel or pathway having any sized- or shaped-cross section or geometry. The sampling channel 3808 can include a protrusion or tang above the displaceable plug 3814, for receiving an holding the displaceable plug 3814 once it is displaced from the second conduit by a pressure differential between the outlet 3804 and the inlet 3802 when the contaminant containment reservoir 3806 receives and contains the first amount of fluid, as will be described in further detail below. Further, the sampling channel 3808 can include one or more blocks, recesses, side channels, cavities, or the like, for receiving the plug 3814.

In some implementations, the housing 3820 can include, or be formed of, a lower housing portion 3822 mated with an upper housing portion 3824, in accordance with an orientation of the device 3800 as shown. The lower housing portion 3822 can include, form, or define the contaminant containment reservoir 3806, the inlet 3802, and a first portion of the first and second conduits. The upper housing portion 3824 can include, form, or define the sampling channel 3808, the outlet 3804, and a second portion of the first and second conduits. The lower housing portion 3822 and upper housing portion 3824 can be mated together and the fluid paths sealed by glue, thermal welding (ultrasonic, laser, friction, etc.), screws, bolts or any other connecting mechanism or process.

As with the device shown in FIGS. 32A and 32B, when a negative pressure differential is applied between the outlet 3804 and the inlet 3802, a first amount of fluid, which is likely to have contaminants, is “pulled” into the inlet 3802 by the negative pressure and into or toward the contaminant containment reservoir 3806, since the sampling channel 3808 is initially blocked or restricted by displaceable plug 3814. And, because of the presence of the displaceable plug 3814 in the second conduit to the sampling channel 3808, the first amount of fluid bypasses the displaceable plug 3814 and the sampling channel 3808. The negative pressure differential will continue to pull fluid into the contaminant containment reservoir 3806 until all the air therein is displaced by fluid, and the fluid contacts the air permeable fluid barrier 3812, effectively sealing it off from the negative pressure.

Once the contaminant containment reservoir 3806 is filled with the first portion of fluid and the air permeable fluid barrier 3812 is sealed, the full pressure differential between the inlet and outlet is applied across the displaceable plug 3814 (similar to what is shown in FIGS. 32A-32C), applying a force to the plug 3814 to deform, collapse inward, loosen and then move it from the second conduit to the sampling channel 3808. Once displaced from the second conduit to the sampling channel 3808, and into the proximal end of the sampling channel 3808, displacement of the displaceable plug 3814 can be maintained by a protrusion or tang on an inside surface of the sampling channel 3808 above the second conduit. Displacement of the displaceable plug 3814 then allows subsequent amounts of fluid to bypass the first amount of fluid, enter into and through the sampling channel 3808, and pulled out the outlet 3804.

A flow or drawing of the subsequent amounts of fluid from the inlet 3802 into and through the sampling channel 3808 work to keep the plug displaced away from the second conduit. For instance, the displaceable plug 3814 can have a bottom surface that is planar and circular, or slightly curved, so as to facilitate displacement from the second conduit. The curvature can be concave or convex. In some implementations, the bottom surface of the displaceable plug 3814 can be coated with a hydrophobic layer, to facilitate flow of the first portion of fluid past the plug 3814, as well as facilitate flow past the plug 3814 and through the sampling channel 3808 when the plug 3814 is displaced.

Although a variety of embodiments have been described in detail above, other modifications are possible. Other embodiments may be within the scope of the following claims.

1. A fluid sample optimization device for optimizing a fluid sample collected by a fluid collection device from a fluid source, a first portion of the fluid sample potentially having contaminants, the fluid sample optimization device comprising:
an inlet configured to connect with the fluid source;
an outlet configured to connect with the fluid collection device;
a sample path connected between the inlet and the outlet;
a contaminant containment reservoir connected between the inlet and the outlet, the contaminant containment reservoir having an air permeable fluid resistor proximate the outlet, the contaminant containment reservoir being arranged to receive, when a pressure differential is applied between the inlet and the outlet, the first portion of the fluid sample from the fluid source to displace air therein through the air permeable fluid resistor and the outlet, such that upon receipt of the first portion of the fluid sample and containment of the contaminants in the contaminant containment reservoir, subsequent portions of the fluid sample can be conveyed by the sample path from the inlet to the outlet when subsequent pressure differentials are applied between the inlet and the outlet; and
a displaceable plug between the inlet and the sample path that is displaced by the subsequent pressure differentials to allow the subsequent portions of the fluid to be conveyed through the sample path.

2. The fluid sample optimization device in accordance with claim 1, further comprising a housing that houses and defines one or more of the inlet, the outlet, the sample path, and the contaminant containment reservoir.

3. The fluid sample optimization device in accordance with claim 1, wherein the air permeable fluid resistor includes a material that seals upon contact with the first portion of the fluid sample.

4. The fluid sample optimization device in accordance with claim 1, wherein the contaminant containment reservoir includes a main basin and a channel connecting the main basin and the inlet.

5. The fluid sample optimization device in accordance with claim 1, wherein each pressure differential is provided by a vacuum pressure from the fluid collection device.

6. A fluid sample optimization device for optimizing a fluid sample collected by a fluid collection device from a fluid source, a first portion of the fluid sample potentially having contaminants, the fluid sample optimization device comprising:
an inlet configured to connect with the fluid source;
an outlet configured to connect with the fluid collection device;
a sample path connected between the inlet and the outlet, the sample path further having a displaceable plug that is configured to inhibit at least a part of the first portion of the fluid sample and the contaminants from entering the sample path; and
a contaminant containment reservoir connected between the inlet and the outlet, the contaminant containment reservoir further having an air permeable fluid resistor proximate the outlet, the contaminant containment reservoir being arranged to receive, when a pressure differential is applied between the inlet and the outlet, the first portion of the fluid sample from the fluid source to displace air therein through the air permeable fluid resistor and the outlet, such that upon receipt of the first portion of the fluid sample and containment of the contaminants in the contaminant containment reservoir, subsequent portions of the fluid sample displace the displaceable plug and are conveyed by the sample path from the inlet to the outlet when subsequent pressure differentials are applied between the inlet and the outlet.

7. The fluid sample optimization device in accordance with claim 6, further comprising a housing that houses and defines one or more of the inlet, the outlet, the sample path, and the contaminant containment reservoir.

8. The fluid sample optimization device in accordance with claim 6, wherein the air permeable fluid resistor includes a material that seals upon contact with the first portion of the fluid sample.

9. The fluid sample optimization device in accordance with claim 6, wherein the contaminant containment reservoir includes a tortuous path.

10. The fluid sample optimization device in accordance with claim 6, wherein each pressure differential is provided by a vacuum pressure provided by the fluid collection device.

11. The fluid sample optimization device in accordance with claim 6, wherein the displaceable plug is friction-fit into a portion of the sample path.

12. The fluid sample optimization device in accordance with claim 11, wherein the portion of the sample path in which the displaceable plug is friction-fit includes a seat.

13. The fluid sample optimization device in accordance with claim 12, wherein the seat comprises an elastomeric O-ring.

14. A fluid sample optimization device for optimizing a fluid sample, a first portion of the fluid sample potentially having contaminants, the fluid sample optimization device comprising:
an inlet;
an outlet;
a contaminant containment reservoir connected between the inlet and the outlet, the contaminant containment reservoir having an air permeable fluid resistor proximate the outlet, the contaminant containment reservoir being arranged to receive, when a pressure differential is applied between the inlet and the outlet, a first portion of the fluid sample to displace air therein through the air permeable fluid resistor and the outlet, such that upon receipt of the first portion of the fluid sample and containment of the contaminants in the contaminant containment reservoir; and
a sample path connected between the inlet and the outlet, the sample path further including a displaceable plug that is configured to inhibit at least a part of the first portion of the fluid sample and the contaminants from entering the sample path during receipt of the first portion of the fluid sample and containment of the contaminants in the contaminant containment reservoir, wherein subsequent portions of the fluid sample are conveyed by the sample path from the inlet to the outlet when subsequent pressure differentials are applied between the inlet and the outlet.

15. The fluid sample optimization device in accordance with claim 14, wherein the displaceable plug is initially secured in the sample path proximate the inlet by an elastomeric seat.

16. The fluid sample optimization device in accordance with claim 14, further comprising a housing that houses and defines one or more of the inlet, the outlet, the sample path, and the contaminant containment reservoir.

17. The fluid sample optimization device in accordance with claim 14, wherein the air permeable fluid resistor includes a material that seals upon contact with the first portion of the fluid sample.

18. The fluid sample optimization device in accordance with claim 14, wherein the contaminant containment reservoir includes a main basin fluidically connected with the inlet by a conduit.

19. The fluid sample optimization device in accordance with claim 15, wherein the displaceable plug is friction-fit into a portion of the sample path.

20. The fluid sample optimization device in accordance with claim 19, wherein the displaceable plug is formed of an elostomeric material.
